(19) United States
(12) Patent Application Publication
(10) Pub. No.: US 2017/0189309 A1
(43) Pub. Date: Jul. 6, 2017

(54) COMPOSITIONS AND METHODS FOR TREATING KERATINOUS SUBSTRATES

(71) Applicant: L'OREAL, Paris (FR)

(72) Inventors: Andrea Michelle ELSEN-WAHRER, Woodbridge, NJ (US); Aditi GOGINENI, Rahway, NJ (US); Charles Michael Sanford SHAW, Madison, NJ (US); Nghi VAN NGUYEN, Edison, NJ (US); Jim Mitchell SINGER, South Orange, NJ (US); XianZhi ZHOU, Millburn, NJ (US)

(21) Appl. No.: 15/396,189

(22) Filed: Dec. 30, 2016

Related U.S. Application Data
(63) Continuation-in-part of application No. 14/985,975, filed on Dec. 31, 2015.

(51) Int. Cl. A61K 8/84; A61K 8/891; A61Q 5/00

(52) U.S. Cl. CPC A61K 8/84; A61Q 5/00; A61K 8/891

(57) ABSTRACT

Treatment compositions including a polycarbodiimide together with a carboxysilicone polymer to enhance the quality of keratinous substrates. Treatment compositions and processes related thereto for treating keratinous substrates, in particular for hair-care, where, according to the process, the polycarbodiimide and carboxysilicone polymer actives may be applied in a treatment that is a single step emulsion or at least two sequential steps. The composition includes 0.1 to 40.0%, by weight, of the composition of a combined amount of the polycarbodiimide and the carboxysilicone polymer in amounts sufficient to impart hydrophobicity to keratinous substrates, including hair fibers, upon application thereto. It is in particular a treatment composition and process for imparting lasting protection and for improving damaged hair by enhancing the hair's hydrophobicity, thereby enhancing retention of curl, making the make hair easier to comb, and improving the feel thereof.

### CROSS-REFERENCE TO RELATED APPLICATION

This patent application is a Continuation-in-Part Application of and claims the benefit of U.S. Non-Provisional patent application Ser. No. 14/985,975 filed on Dec. 31, 2015, and entitled “COMPOSITIONS AND METHODS FOR TREATING KERATINOUS SUBSTRATES,” the disclosure of which is incorporated by reference as if fully rewritten herein.

### FIELD OF THE INVENTION

The present invention relates to a composition and method for treating keratinous substrates. More particularly, the present invention relates to keratinous substrate treatment compositions having a polycarbodiimide compound, and, in certain embodiments, at least one carboxysilicone polymer, for imparting and improving hydrophobicity to the keratinous substrate to thereby achieve one or more of repairing, providing protection from extrinsic damage, enhancing durable non-permanent shaping, and enhancing durable retention of a non-permanent shape of the keratinous substrate, in particular, keratinous fibers, and more particularly, hair.

### BACKGROUND OF THE INVENTION

The appearance and/or condition of keratinous substrates, for example, keratinous fibers such as hair, skin, nails, and lips, are often affected by both extrinsic and intrinsic factors such as aging. In particular, when keratinous substrates are exposed to environmental conditions, for example, high or low humidity or to ultraviolet radiation from the sun, these substrates can lose many of their desirable properties and even become damaged. Keratinous fibers, especially hair, are constantly exposed to harsh extrinsic conditions, such as sun, chemical damage, e.g., from detergents, bleaching, relaxing, dyeing, and permanent waving, heat, e.g., from hair dryers or curlers, and mechanical stress or wear, e.g., from brushing or grooming activities. In addition, any type of hair can diminish in quality and/or quantity over time by age and/or due to factors such as natural greasiness, sweat, shedded skin cells from the scalp, pollution, dirt, and extreme humidity conditions.

The above-described factors can result in thinning hair and/or harm the visual appearance and the feel of the hair, and lead to lank body and decreased volume. For example, hair can dry out and lose its shine or color or become frizzy and less manageable under low and high humidity conditions. Under low humidity conditions, hair can dry out and dried-out hair tends to be less shiny and more brittle. Conversely, under high humidity conditions, hair tends to absorb water, causing hair to lose its shape and become unmanageable and unattractive. Furthermore, hair can lose its desirable attributes due to physical stress on the hair such as brushing and application of heat. The magnitude of the consequences of these factors is variable, depending on, for example, the quality of the hair, length, style, and environmental factors. As such, these factors generally result in damage to the keratinous fibers, either by affecting protective materials on the surface of the hair (the cuticle), or by altering the hair fiber internally (the cortex).

More specifically, extrinsic conditions may strip protective materials from the surface of the hair, and/or they may disrupt the organized structure of the hair fibers, called the a-structure, which may be accompanied by a decrease in the tensile strength. Such damage to hair by extrinsic factors is more evident the further the hair fiber has grown from the root, because the hair has been exposed longer to such extrinsic factors. In effect, the hair has what may be called a “damage history” as it grows, i.e., the further from the root, the lower the tensile strength and the greater the breakdown in α-structure that has occurred. As a result, consumers continue to seek products such as hair care and hair cosmetic compositions which protect and enhance the appearance of hair as well as reduce the deleterious effects of adverse environmental conditions, photo-damage, and physical stress.

Morphologically, a hair fiber contains four structural units: cuticle, cortex, medulla, and intercellular cement. Robbins, C. R. Chemical and Physical Behavior of Human Hair, 3rd Edition, Springer-Verlag (1994). The cuticle layers are located on the hair surface and consist of flat overlapping cells (“scales”). These scales are attached at the root end and point toward the distal (tip) end of the fiber and form layers around the hair cortex. The cortex comprises the major part of the hair fiber. The cortex consists of spindle-shaped cells, or macrofibrils, that are aligned along the fiber axis. The macrofibrils further consist of microfibrils (highly organized protein units) that are embedded in the matrix of amorphous protein structure. The medulla is a porous region in the center of the fiber. The medulla is a common part of wool fibers but is found only in thicker human hair fibers. Finally, the intercellular cement is the material that binds the cells together, forming the major pathway for diffusion into the fibers.

The mechanical properties of hair are determined by the cortex. A two-phase model for the cortex organization has been suggested. Milczarek et al, Colloid Polym. Sci., 270, 1106-1115 (1992). In this model, water-impenetrable microfilaments (“rods”) are oriented parallel with the fiber axis. The microfilaments are embedded in a water-penetrable matrix (“cement”). Within the microfilaments, coiled protein molecules are arranged in a specific and highly organized way, representing a degree of crystallinity in the hair fiber.

Similar to other crystalline structures, hair fibers display a distinct diffraction pattern when examined by wide-angle X-ray diffraction. In normal, non-stretched hair fibers this pattern is called an “alpha-pattern”. The alpha-pattern or a-structure of hair is characterized by specific repeated spacings (9.8 Å, 5.1 Å, and 1.5 Å). All proteins that display this X-ray diffraction pattern are called a-proteins and include, among others, human hair and nails, wool, and porcupine quill. When the hair fiber is stretched in water, a new X-ray diffraction pattern emerges that is called a “β-pattern”, with new spacings (9.8 Å, 4.65 Å, and 3.3 Å).

Damage to hair may occur in the cuticle and/or the cortex. When normal hair is damaged by heat, chemical treatment, UV radiation, and/or physical/mechanical means, myriad chemical and physical changes are induced in the hair. For example, these damaging processes have been known to produce removal or damage to cuticle scales or to cleave the thioester linkage holding the hydrophobic 18-methyl eicosanoic acid (“18-MEA”) layer to hair. Thus, it is commonly observed that undamaged hair exhibits significant hydrophobic character, whereas damaged hair shows significant hydrophilic character due to the removal of surface lipids.

There is a need, therefore, for cosmetic products that are useful in protecting the chemical and physical structure of keratinous fibers from harsh extrinsic conditions and restoring the hair's physical properties to undamaged states following damage by extrinsic conditions. More particularly, there is a need to find materials or compositions or methods that can provide a water-resistant and/or hydrophobic and/or protective barrier to hair to protect it at the cortex. Such a protective barrier should not be easily transferred from the substrate over time by normal everyday activity. In addition, the protective barrier should be shampoo, wash or water-resistant so that the barrier is not easily removed. Non-transfer and shampoo, wash or water-resistant cosmetic, hair and skin care compositions are sought which have the advantage of forming a deposit which does not undergo even partial transfer to the substrates with which they are brought into contact (for example, clothing). It is also desirable to have compositions that do not easily “run off” or wash off the skin and lips when exposed to water, rain or tears. Accordingly, a product that imparts a protective barrier to the substrate that also is shampoo, wash or water resistant and non-transferable would be of benefit to the area of cosmetic products. As such, makers of cosmetic products such as hair and skin care products continue to seek materials and ingredients that can provide such benefits.

In addition, in today's market, many consumers prefer the flexibility of non-permanent hairstyles, that is, those styles obtained via non-permanent shaping of the hair. Typically, such non-permanent styles disappear when the hair is wetted, especially when the hair is washed with water and/or shampoo or when the hair is exposed to high humidity conditions. Methods for non-permanent shaping of keratinous fibers include, for example, brushing, teasing, braiding, the use of hair rollers, and heat styling, optionally with a commercially available styling product. Non-limiting examples of heat styling include blow drying, crimping, curling, and straightening methods using elevated temperatures (such as, for example, setting hair in curlers and heating, and curling with a curling iron and/or hot/steam rollers and/or flat iron).

While such compositions and methods may provide for non-permanent shaping of keratinous fibers, many consumers also desire longer lasting or durable styling/shaping than most known materials (e.g., film-forming agents, resins, gums, and/or adhesive polymers), commercially available products (e.g., conventional hair sprays, mousses, gels and lotions), and methods employing these materials and products provide. For example, many consumers desire compositions and methods that improve and preserve non-permanent curl formation or hair style.

There is a need, therefore, for materials, compositions and methods that result in more durable or longer lasting shape or style even when the styled/shaped/curled hair is exposed to adverse environmental and physical factors and/or when wetted, washed, or shampooed.

To achieve at least one of these and other advantages, the present invention provides a method of protecting and/or repairing a keratinous substrate, and more particularly, a keratinous fiber chosen from hair, eyelashes and eyebrows from extrinsic damage caused by heating, UV radiation or chemical treatment by applying to said keratinous fiber a composition that includes a polycarbodiimide compound and a carboxysilicone polymer in an amount effective to confer or improve the keratinous fiber's hydrophobicity.

### BRIEF SUMMARY OF THE INVENTION

In an exemplary embodiment, a keratinous treatment composition including a polycarbodiimide compound and a carboxysilicone polymer is disclosed. The composition includes about 0.1 to about 40.0% by weight, based on the total weight of the composition, of a combined amount of the polycarbodiimide compound and the carboxysilicone polymer. The composition includes amounts of each of the polycarbodiimide compound and the carboxysilicone polymer sufficient to impart one or more of hydrophobicity or increased hydrophobicity to the keratinous substrate after application thereto and increased hold to the shape or configuration of the keratinous substrate.

In another exemplary embodiment, a method of protecting a keratinous fiber chosen from hair, eyelashes and eyebrows from extrinsic damage caused by heating, UV radiation or chemical treatment, or of repairing a keratinous fiber chosen from hair, eyelashes and eyebrows following extrinsic damage caused by heating, UV radiation or chemical treatment. The method includes applying to the keratinous fiber a composition including the polycarbodiimide and carboxysilicone polymer in an amount effective to protect or repair the keratinous fiber; wherein the polycarbodiimide compound is present at a concentration of from about 0.1 to about 40.0% by weight, based on the total weight of the composition.

Another embodiment of the present invention is a method protecting a keratinous fiber chosen from hair comprising applying to the keratinous fiber the composition of the present invention in an amount effective to protect or repair said keratinous fiber before or during or after chemically treating the hair (e.g., dyeing the hair using permanent, semi-permanent or demi-permanent dyeing compositions, bleaching/lightening or lifting the color of hair by chemical oxidizing agents, perming the hair using chemical reducing/oxidizing agents, relaxing the hair using lye and no-lye compositions, straightening the hair using chemical straightening agents).

In some embodiments, the keratinous fiber in the above described composition is heated and the composition is applied to the fiber prior to heating or during heating or after heating the fiber.

In another exemplary embodiment, a method for durable non-permanent shaping of at least one keratinous fiber or for durable retention of a non-permanent shape of at least one keratinous fiber is disclosed. The method includes applying to the at least one keratinous fiber the composition including the polycarbodiimide and carboxysilicone polymer.

In some embodiments, the above-described method includes a step of heating the keratinous fiber prior to or during or after the application of the above-described composition.

The present invention is also directed to a method for cosmetic treatment of keratinous tissues, such as keratinous fibers, by applying the above-disclosed composition onto a surface of the keratinous tissue, such as the cuticle of hair fibers.

The present invention is also directed to methods for applying the above-disclosed composition onto a surface of the keratinous tissue, such as the cuticle of hair fibers in a step wise fashion, the steps including a one step process and a multi-step process including two, three or more steps, and combinations of these.

According to some such embodiments, the method includes a one-step process, wherein a composition including the polycarbodiimide, carboxysilicone polymer, and solvent is prepared and applied onto the keratinous substrate.

According to other embodiments, the method includes a two-step application process, wherein individual components (polycarbodiimide combined with a solvent and carboxysilicone polymer combined with a solvent) are applied to the keratinous substrate in a step-wise fashion in any order to treat the substrate. In one such embodiment, the keratinous substrate is first treated with the polycarbodiimide phase, followed by treatment with the carboxysilicone polymer phase.

According to other embodiments, the method includes a reverse-step system, wherein compositions comprising at least one polycarbodiimide and at least one carboxysilicone polymer are prepared as separate phases, and each phase is applied separately to the keratinous substrate, such as hair. In one such embodiment, the keratinous substrate is first treated with the carboxysilicone polymer phase, followed by treatment with the polycarbodiimide phase.

According to yet other embodiments, the method includes a three-step application process, wherein individual components (polycarbodiimide combined with a solvent and carboxysilicone polymer combined with a solvent) are applied to the keratinous substrate in a step-wise fashion in any order to treat the substrate. In one such embodiment, the keratinous substrate is first treated with the carboxysilicone polymer phase, followed by treatment with the polycarbodiimide phase, followed by treatment with the carboxysilicone polymer phase.

The present invention is also directed to methods and kits for cosmetic treatment of keratinous tissues, such as keratinous fibers, by applying the above-disclosed composition onto a surface of the keratinous tissue, such as the cuticle of hair fibers in a step wise fashion. According to some such embodiments, the kit includes separate packaging of one or more of the actives of the inventive composition provided in one or more of thickened or un-thickened aqueous and non-aqueous phases, and packaging of any of the foregoing with one or more of processing agents selected from a coloring agent, a pigmenting agent, a permanent process agent, a relaxing process agent, a straightening process agent, and a highlighting process agent.

Other features and advantages of the present invention will be apparent from the following more detailed description of the exemplary embodiment which illustrates, by way of example, the principles of the invention.

### DETAILED DESCRIPTION OF THE INVENTION

Other than in the operating examples, or where otherwise indicated, all numbers expressing quantities of ingredients and/or reaction conditions are to be understood as being modified in all instances by the term “about,” meaning within 10% of the indicated number (e.g. “about 10%” means 9%-11% and “about 2%” means 1.8%-2.2%).

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used. The adjective “any” means one, some, or all indiscriminately of whatever quantity.

“Active material” as used herein with respect to the percent amount of an ingredient or raw material, refers to 100% activity of the ingredient or raw material.

As used herein, the terms “applying a composition onto keratin fibers” and “applying a composition onto hair” and variations of these phrases are intended to mean contacting the fibers or hair, with at least one of the compositions of the invention, in any manner.

“At least one,” as used herein, means one or more and thus includes individual components as well as mixtures/combinations.

The term “comprising” (and its grammatical variations) as used herein is used in the inclusive sense of “having” or “including” and not in the exclusive sense of “consisting only of.”

“Conditioning,” as used herein, means imparting at least one of combability, manageability, moisture-retentivity, luster, shine, softness, and body to the hair.

“Durable conditioning,” as used herein, means that, following at least one shampoo/washing/rinsing after treatment of keratinous fibers such as hair with the compositions of the present disclosure, treated hair still remains in a more conditioned state as compared to untreated hair. The state of conditioning can be evaluated by measuring, and comparing, the ease of combability of the treated hair and of the untreated hair in terms of combing work (gm-in) and/or the substantivity of the conditioning agent on the hair and/or the hydrophobicity of hair which can be assessed by contact angle measurements (spread of a water droplet on the surface of the hair).

“Durable retention of a shape,” as used herein, means that, following at least one shampoo/washing/rinsing after treatment of keratinous fibers such as hair with the compositions of the present disclosure, treated hair still exhibits the ability to retain a particular or desirable shape after styling as compared to the exhibited ability of untreated hair to retain a particular or desirable shape after styling. “Durable retention of a shape” can also be related to the hydrophobicity of hair which can be assessed by contact angle measurements (spread of a water droplet on the surface of the hair).

“Durable shaping,” as used herein, refers to holding or keeping a shape of a keratinous fiber until the keratinous fiber is washed with water and/or shampoo. Retention of a shape can be evaluated by measuring, and comparing, the ability to retain a curl under conditions of high relative humidity of the treated hair and of the untreated hair in terms of Curl Efficiency. “Durable shaping” can also be related to imparting hydrophobicity to hair which can be assessed by contact angle measurements (spread of a water droplet on the surface of the hair).

“Heating” refers to the use of elevated temperature (i.e., above room temperature such as above 40° C.). In one embodiment, the heating in the inventive method may be provided by directly contacting the at least one keratinous fiber with a heat source, e.g., by heat styling of the at least one keratinous fiber. Non-limiting examples of heat styling by direct contact with the at least one keratinous fiber include flat ironing and curling methods using elevated temperatures (such as, for example, setting hair in curlers and heating, and curling with a curling iron and/or hot rollers). In another embodiment, the heating in the inventive method may be provided by heating the at least one keratinous fiber with a heat source which may not directly contact the at least one keratinous fiber. Non-limiting examples of heat sources which may not directly contact the at least one keratinous fiber include blow dryers, hood dryers, heating caps and steamers.

“A heat-activated” composition, as used herein, refers to a composition which, for example, shapes the at least one keratinous fiber better than the same composition which is not heated during or after application of the composition. Another example includes a composition which retains a shape of at least one keratinous fiber better than the same composition which is not heated during or after application.

“High humidity,” as defined herein, refers to atmospheric humidity above 40%.

“Homogeneous” means having the visual appearance of being substantially uniform throughout, i.e., visually appears as a single-phase emulsion and/or dispersion.

“Keratinous substrate,” as used herein, includes, but is not limited to, skin, hair, and nails. “Keratinous substrate” as used herein also includes “keratinous tissue” or “keratinous fibers,” which as defined herein, may be human keratinous fibers, and may be chosen from, for example, hair, such as hair on the human head, or hair comprising of eyelashes or hair on the body.

The term “style” or styling” as used herein includes shaping, straightening, curling, or placing a keratin fiber such as hair, in a particular arrangement, form or configuration; or altering the curvature of a keratinous fiber or other substrate; or re-positioning a keratin fiber or other substrate to a different arrangement, form or configuration; or providing/maintaining a hold to the shape or configuration of the keratin fiber. In some embodiments, the hold to the shape of configuration of the fiber may be expressed as an improved bending force property.

As used herein, the terms “styling keratinous fibers” and variations thereof are understood to refer to any means or method of modifying the appearance of the keratinous fibers or the hair with respect to their spatial arrangement or configuration or curvature or form. When the keratinous fibers comprise hair on the human head, the term “styling keratinous fibers” or “styling hair” is also understood to include curling or waving or embossing the hair or smoothing or straightening the hair, or spiking the hair or providing/maintaining a hold to the shape or configuration of the keratin fiber.

The term “treat” (and its grammatical variations) as used herein refers to the application of the compositions of the present invention onto keratinous substrates such as keratinous fibers or hair or skin.

The term “wash cycle” as used herein, refers to a step or process of washing a keratinous substrate and may include treating the substrate with a surfactant-based product (e.g., shampoo or conditioner or body wash) then washing or rinsing the substrate with water. The term “wash cycle” may also include washing or rinsing the substrate with water.

Referred to herein are trade names for materials including, but not limited to polymers and optional components. The inventors herein do not intend to be limited by materials described and referenced by a certain trade name. Equivalent materials (e.g., those obtained from a different source under a different name or catalog (reference number) to those referenced by trade name may be substituted and utilized in the methods described and claimed herein.

All percentages and ratios are calculated by weight unless otherwise indicated. All percentages are calculated based on the total weight of a composition unless otherwise indicated. All component or composition levels are in reference to the active level of that component or composition, and are exclusive of impurities, for example, residual solvents or by-products, which may be present in commercially available sources.

It is an object of the present invention to provide materials and compositions and methods which provide both a protective barrier onto keratinous substrates such as hair and which impart native/undamaged physical properties—such as hydrophobicity, ease of combing, etc.—to hair, in particular, damaged hair, as well as impart durable or long lasting physical properties mimicking natural/undamaged hair to treated hair. It is also an object of the present invention to provide materials and compositions and methods which improve the shampoo, wash or water resistance of cosmetic and personal care compositions.

It has been surprisingly and unexpectedly discovered by the inventors that a composition containing the combination of silicone polymers containing at least one carboxylic acid group, or carboxysilicone polymers, and polycarbodiimides for cosmetic application, when applied to keratinous substrates such as hair, enhance the properties (hydrophobicity, adhesion, chemical resistance, water resistance etc.) and deliver superior performance to the substrate. Application of the actives may be in a mixture, or sequentially, or combinations of these. In embodiments of this disclosure, the combination of polycarbodiimide compounds with carboxysilicone polymers enhance the properties of hair wherein the combination increases the conditioning effect (e.g., hydrophobicity, shine and smoothness), strengthens the hair, increases the stiffness and humidity resistance of hair and ameliorates the condition of damaged hair by improving the appearance and quality of hair (for example, smoother feel, softer feel, less frizzy, less dry, more discipline). In addition, the composition, according to the present disclosure, provides a significant reduction in combing force which indicates that hair is easier to comb and/or has less tangling. The compositions according to the present disclosure, imparted increased percent curl retention on curled/shaped hair indicating higher resistance of the hair to high humidity and high temperature as compared to compositions with either material alone. In addition, the composition, according to the present disclosure, may be applied in a one-step or multiple-step process such as a two-step process, or a three-step process, or a reverse process wherein the order of any multistep process may be varied.

It has also been surprisingly and unexpectedly discovered by the inventors that sequential application of each of the actives selected from one or more polycarbodiimides and carboxysilicone polymers, in some embodiments, provides enhanced results as compared to application of some combinations of the two actives together when applied to keratinous substrates comprising hair. The benefits thus conferred by such sequential application include reduction of friction of the hair cuticle, and extended resistance to breakage over up to 10 wash cycles. These benefits provide smoother feel, enhanced resistance to combining of wet and dry hair, extended bounce and shine, and visibly minimized dry ends.

Without being bound to any one theory, the inventors of the present disclosure believe that the polycarbodiimide compound and the carboxysilicone polymer comprising the keratinous treatment compositions of the invention react to each other and to the keratin substrate when such compositions are applied onto keratinous substrates such as hair or skin. It is also believed that the compositions of the present disclosure provide a protective barrier useful in cosmetic applications such as hair care, hair styling, nail care, makeup, skin care, and sun care products such that the hydrophobicity of the keratinous substrates is improved or restored resulting in significantly better cosmeticities, feel and appearance, and less damaged condition of the substrates such as hair and skin.

The compositions according to the invention, are compositions including polycarbodiimide and carboxysilicone polymers. The composition may include other suitable ingredients for hair treatment or hair repair. For example, known solvents and/or additives may be utilized in addition to the polycarbodiimide and carboxysilicone polymers to provide additional benefits to the composition. When both polycarbodiimide and carboxysilicone are utilized in combination to form the inventive composition, a significant increase in hydrophobicity of keratinous fiber is provided. The range of concentrations by weight of the composition over which the association provides hydrophobicity to and/or hold to the shape or configuration of keratinous substrates is about 0.1% to about 40% total actives. A range of ratios polycarbodiimide to carboxysilicone polymer of about 1:10 about 10:1 or about 1:5 to about 5:1, including all ranges and subranges there-between, or such as about 1:1 or about 1:2 or about 1:3 or about 1:4 or about 1:5 or about 1:7 or about 1:7 or about 1:8 or about 1:9 or about 1:10 or about 10:1 or about 9:1 or about 8:1 or about 7:1 or about 8:1 or about 7:1 or about 6:1 or about 5:1 or about 4:1 or about 3:1 or about 2:1.

In accordance with the various embodiments, the combination of polycarbodiimide compound and the carboxysilicone polymer is present in the compositions according to the disclosure in amounts range from about 0.1% to about 40%, and in some embodiments from about 0.01% to about 20%, and in some further embodiments from about 1% to about 10%. In some representative embodiments, the combination of polycarbodiimide compound and the carboxysilicone polymer is present from about 0.5% to about 10%, and from about 0.5% to about 5%. In yet other embodiments, the combination of polycarbodiimide compound and the carboxysilicone polymer is present from at least 0.5%. And in yet other embodiments, the combination of polycarbodiimide compound and the carboxysilicone polymer is present from at least 0.5% to up to about 40%. In some specific embodiments, the combination of polycarbodiimide compound and the carboxysilicone polymer is present at about 5%. In some other specific embodiments, the combination of polycarbodiimide compound and the carboxysilicone polymer is present at about 2.5%. In some other specific embodiments, the combination of polycarbodiimide compound and the carboxysilicone polymer is present at about 0.75%.

Thus, the combination of polycarbodiimide compound and the carboxysilicone polymer may be present from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 to about 40 percent by weight, including increments and ranges therein and there between.

Polycarbodiimide Compounds

A carbodiimide group is a linear triatomic moiety generally depicted by Formula (I):

*—(N═C═N)—* (I)

At least one of the nitrogens is linked to or incorporated into a backbone or other bridging group to result in a molecule having at least two carbodiimide groups.

Polycarbodiimides

In one embodiment, the polycarbodiimides comprising of at least two carbodiimide units, as described above, can be represented by Formula (II):

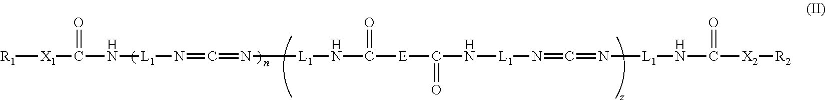

wherein X₁ and X₂ each independently represent O, S or NH. R₁ and R₂ are selected from a hydrocarbon group containing one or more catenary or non-catenary hetero-atoms, such as nitrogen, sulfur and oxygen, and linear or branched and cyclic or acyclic groups which can be ionic or non-ionic segments, or a partially or fully fluorinated hydrocarbon group that may contain one or more catenary or non-catenary hetero-atoms; n and z are, each independently, an integer of 0 to 20; L₁ (Linker of carbodiimide groups) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group, and a C₃ to C₁₂ divalent heterocyclic group; wherein a plurality of L₁s may be identical to or different from one another, and wherein in another embodiment, L₁ of formula (II) is selected from a C₁ to C₁₈ divalent aliphatic hydrocarbon group, a C₃ to C₁₃ divalent alicyclic hydrocarbon group, a C₆ to C₁₄ divalent aromatic hydrocarbon group that is not chosen from m-tetramethylxylylene, and a C₃ to C₁₂ divalent heterocyclic group; wherein a plurality of L₁s may be identical to or different from one another;

wherein E is a radical selected from the following formulas:

O—R₃—O; S—R₄—S; and

R₅—N—R₄—N—R₅;

wherein R₃ and R₄ are each independently hydrocarbon radicals that may contain halogen atoms or one or more catenary (i.e.; in chain, bonded only to carbon) or non-catenary hetero atoms, including an aromatic, cycloaliphatic, aryl and alkyl radical (linear or branched) and R₅ is hydrogen, or a hydrocarbon radical which can contain halogen atoms or one or more catenary (i.e.; in chain, bonded only to carbon) or non-catenary hetero atoms.

Examples of R₁ and R₂ can be methyl glycolate, methyl lactate, polypropylene glycol, polyethylene glycol monomethyl ether, dialkylamino alcohol.

Examples of L₁ can be the diradical of tolylene, hexamethylene, hydrogenated xylylene, xylylene, 2,2,4-trimethylhexamethylene, 1,12-dodecane, norbornane, 2,4-bis-(8-octyl)-1,3-dioctylcyclobutane, 4,4′-d icyclohexylmethane, tetramethylxylylene, isophorone, 1,5-naphthylene, 4, 4′ diphenylmethane, 4, 4′ diphenyldimethylmethane, phenylene.

Polycarbodiimides may include polymers with a plurality of carbodiimide groups appended to the polymer backbone. For example, U.S. Pat. No. 5,352,400 (the disclosure of which is incorporated by reference herein for all purposes as if fully set forth) discloses polymers and co-polymers derived from alpha-methylstyryl-isocyanates. Such a polymer is illustrated in Formula (III).

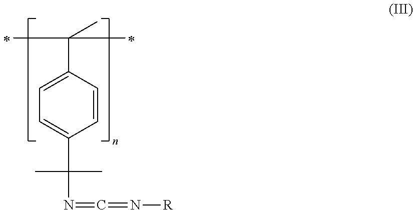

wherein R is an alkyl, cycloalkyl or aryl group (in some particular embodiments having from 1 to 24 carbon atoms).

In another embodiment, polycarbodiimides, according to the present disclosure, include polycarbodiimides having branched structures, like that shown in Formula (IV), and as described in Chapter 8 of Technology for Waterborne Coatings, E. J. Glass Ed., ACS Symposium 663, 1997; The Application of Carbodiimide Chemistry to Coating, by J. W. Taylor and D. R. Bassett (the disclosure of which is incorporated by reference herein for all purposes as if fully set forth).

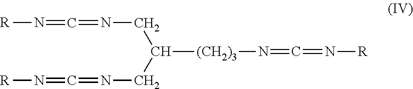

wherein R is an alkyl, cycloalkyl or aryl group (in some particular embodiments having from 1 to 24 carbon atoms).

In one embodiment, the compositions of the present disclosure does not employ a polycarbodiimide having a linker L₁ chosen from m-tetramethylxylylene.

Suitable polycarbodiimide compounds include, but are not limited to, those commercially sold by the suppliers Nisshinbo, Picassian, and 3M. Particularly suitable polycarbodiimide compounds include, but are not limited to, those known by the name under the CARBODILITE series, V-02, V02-L2, SV-02, E-02, V-10, SW-12G, E-03A, commercially sold by Nisshinbo.

In some embodiments, the polycarbodiimide of the present disclosure is selected from compounds of formula (II) wherein L₁ (Linker of carbodiimide groups) represents a C₁ to C₁₈ divalent aliphatic hydrocarbon group, a C₃ to C₁₃ divalent alicyclic hydrocarbon group, a C₃ to C₁₂ divalent heterocyclic group, or a C₆ to C₁₄ divalent aromatic hydrocarbon group;

wherein a plurality of L₁s may be identical to or different from one another.

In other embodiments, the polycarbodiimide of the present disclosure is selected from compounds of formula (II) wherein L₁ is not chosen from m-tetramethylxylylene.

In certain embodiments, the composition of the present disclosure is devoid of a polycarbodiimide that has a linker L₁ chosen from m-tetramethylxylylene.

The polycarbodiimide is typically present in the composition of the present disclosure in an amount of from about 0.01% to about 20%, by weight, in some particular embodiments from about 0.5% to about 10%, by weight, and from about 0.5% to about 5.0%, by weight, including all ranges and subranges therebetween, based on the total weight of the composition.

In accordance with the various embodiments, the polycarbodiimide compound is present in the compositions according to the disclosure in amounts range from about 0.01% to about 20%, and in some embodiments from about 0.1% to about 15%, and in some further embodiments from about 0.1% to about 10%. In some representative embodiments, the polycarbodiimide compound is present from about 0.1% to about 5%, and from about 0.2% to about 3%. In yet other embodiments, the polycarbodiimide compound is present from at least 0.2%. And in yet other embodiments, the polycarbodiimide compound is present from at least 0.2% to up to about 40%. In some specific embodiments, the polycarbodiimide compound is present at about 1.5%. In some specific embodiments, the polycarbodiimide compound is present at about 0.75%. In some other specific embodiments, the polycarbodiimide compound is present at about 0.25%.

Thus, the polycarbodiimide compound may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and there between.

Silicone Polymers Having At Least One Carboxylic Acid Group (Carboxysilicone Polymers).

The silicone polymers having at least one carboxylic acid group, referred herein as carboxysilicone polymers, according to the present disclosure, may be an organopolysiloxane comprising:

(A) a compound having the following formula (VI):

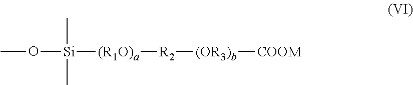

wherein R₁ and R₃ independently denote a linear or branched alkylene radical containing from 2 to 20 carbon atoms and R₂ denotes a linear or branched alkylene radical containing from 1 to 50 carbon atoms which can comprise a hydroxyl group, a represents 0 or 1, b is a number ranging from 0 to 200 and M denotes hydrogen, an alkali metal or alkaline-earth metal, NH4 or a quaternary ammonium group, such as a mono-, di-, tri- or tetra(C1-C4 alkylammonium) group, R₁ and R₃ can denote, for example, ethylene, propylene or butylene, or

(B) a group comprising at least one pyrrolidone carboxylic acid unit having the following formula (VII):

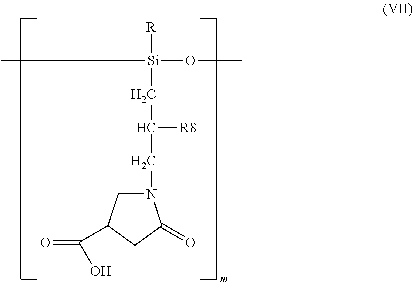

in which R is selected from methyl or phenyl; R8 is hydrogen or methyl, m is an integer from 1 to 1000, or

(C) a group comprising at least one polyvinyl acid/ester unit (C) resulting from the polymerization of Divinyl-PDMS, Crotonic Acid, Vinylacetate, and Vinyl Isoalkylester,

and combinations of (A), (B) and (C).

Suitable carboxysilicone polymers include, for example, a silicone polymer comprising at least one carboxylic acid group chosen from organopolysiloxanes of formula (VIII):

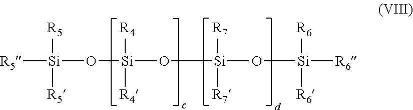

wherein the radicals R4, R4′ are identical to or different from each other and are chosen from a linear or branched C1-C22 alkyl radical, a C1-C22 alkoxy radical and a phenyl radical, the radicals R5, R5′, R5″, R6, R6′, R6″, R7, and R7′ are identical to or different from each other and are chosen from a linear or branched C1-C22 alkyl radical, a C1-C22 alkoxy radical, a phenyl radical, a radical —(R₁O)a-R2-(OR3)b-COOM, a radical containing pyrrolidone carboxylic acid, a radical of polyvinyl acid/ester; and

wherein at least one of the radicals R5, R6 and R7 is a radical chosen from a radical —(R1O)a-R2-(OR3)b-COOM, a radical containing pyrrolidone carboxylic acid, a radical of polyvinyl acid/ester;

wherein R1, R2, R3, a, b and M have the same meaning as described in Unit (A) above;

wherein c and d are integers from 0 to 1000, the sum c+d in some particular embodiments ranging from 1 to 1000 or from 2 to 1000.

Among the carboxysilicone polymers of formula (VIII) that comprise at least one unit (VI), which in some particular embodiments are the compounds of formula (IX) below:

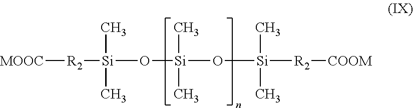

wherein R2, and M have the same meaning as described in Unit (A) above, n is an integer from 1 to 1000. Examples of compound (IX) are: dual-end carboxy silicones X-22-162C from Shin Etsu and Silform INX (INCI name: Bis-Carboxydecyl Dimethicone) from Momentive.

Other exemplary embodiments organopolysiloxanes of formula (VIII) are the ones of formula (X):

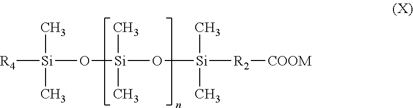

in which R2, R4, n, and M having the same meaning as in Unit (I) above. An example of compound (X) is a single-end carboxy silicone X-22-3710 from Shin Etsu.

Other exemplary embodiments organopolysiloxanes of formula (VIII) are the ones of formula (XI):

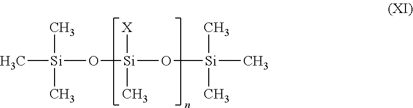

wherein X is a radical —(R1O)a-R2-(OR3)b-COOM wherein R1, R2, R3, a, b and M have the same meaning as described in Unit (A) above.

Even more particularly, the compounds of formula (XI) in which a and b are equal to 0 and R₂ is a linear or branched C₂-C₁₂ alkylene group such as (CH₂)₉, (CH₂)₁₀ or —CH(CH₃)— are exemplary embodiments. An example of compound (XI) is a side-chain carboxy silicone X-22-3701E from Shin Etsu.

Among the organopolysiloxanes of formula (VIII) that contain unit (B), exemplary embodiments include the compounds of formula (XII) below:

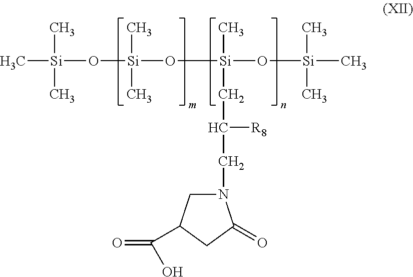

wherein R8, m, are defined as in Unit (B) above and n is an interger from 1 to 1000. An example of compound (XII) is Grandsil PCA such as in Grandsil SiW-PCA-10 (INCI name: Dimethicone (and) PCA Dimethicone (and) Butylene Glycol (and) Decyl Glucoside from Grant Industries.

Among the organopolysiloxanes of formula (VIII) that contain polyvinyl acid/ester Unit (C), exemplary embodiments are crosslinked anionic copolymers comprised of organic polymer blocks and silicone blocks, resulting in a multiblock polymer structure. In particular, the silicone-organic polymer compound of the present invention may be chosen from crosslinked anionic copolymers comprising at least one crosslinked polysiloxane structural unit. An example of such a branched multi-block carboxysilicone polymer is Belsil® P1101 (may also be known under the tradename Belsil® P1101) (INCI name: Crotonic AcidNinyl C8-12 Isoalkyl Esters/VA/Bis-Vinyldimethicone Crosspolymer, also known by the technical name of Crotonic Acid/Vinyl C8-12 Isoalkyl Esters/VA/divinyldimethicone Crosspolymer) from Wacker Chemie AG.

Additional suitable carboxysilicone polymers are described, for example, in patent applications WO 95/23579 and EP-A-0,219,830, which are hereby incorporated by reference in their entirety.

Compounds corresponding to formula (XI) above are sold, for example, under the name HUILE M 642 by the company Wacker, under the names SLM 23 000/1 and SLM 23 000/2 by the company Wacker, under the name 176-12057 by the company General Electric, under the name FZ 3703 by the company OSI and under the name BY 16 880 by the company Toray Silicone.

Other non-limiting examples of carboxysilicone polymers are silicone carboxylate containing polymers (silicone carboxylates).

Suitable silicone carboxylates may be chosen from water soluble silicone compounds comprising at least one carboxylic acid group, oil soluble silicone compounds comprising at least one carboxylic acid group, water-dispersible silicone compounds comprising at least one carboxylic acid group, and silicone compounds comprising at least one carboxylic acid group which are soluble in organic solvents. In one embodiment, the silicone carboxylate further comprises at least one alkoxylated chain, wherein the at least one alkoxy group may be chosen from terminal alkoxy groups, pendant alkoxy groups, and alkoxy groups which are intercalated in the skeleton of the at least one silicone compound. Non-limiting examples of at least one alkoxy group include ethylene oxide groups and propylene oxide groups.

The at least one carboxylic acid group may be chosen from terminal carboxylic acid groups and pendant carboxylic acid groups. Further, the at least one carboxylic acid may be chosen from carboxylic acid groups in free acid form, i.e., —COOH, and carboxylic acid groups in salt form, i.e., —COOM, wherein M may be chosen from inorganic cations, such as, for example, potassium cations and sodium cations, and organic cations.

In one embodiment, the silicone carboxylate is a compound of formula (XIII):

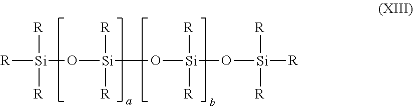

wherein a is an integer ranging from 1 to 100; b is an integer ranging from 0 to 500; and R, which may be identical or different, are each chosen from optionally substituted hydrocarbon groups comprising from 1 to 9 carbon atoms, optionally substituted phenyl groups, and groups of the following formula (XIV):

—(CH₂)₃—O—(EO)_{c}—(PO)_{d}—(EO)ₑ—C(O)—R′—C(O)—OH (XIV)

wherein c, d, and e, which may be identical or different, are each integers ranging from 0 to 20; EO is an ethylene oxide group; PO is a propylene oxide group; and R′ is chosen from optionally substituted divalent hydrocarbons, such as alkylene groups and alkenylene groups comprising from 2 to 22 carbon atoms, and optionally substituted divalent aromatic groups, such as groups of the following formula:

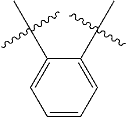 [SMILES: *c1ccccc1*]

and groups of the following formula:

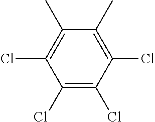

with the proviso that at least one of the R groups is chosen from groups of formula (XIV) and with the further proviso that when only one of the R groups is chosen from groups of formula (XIV), the other R groups are not all methyl groups.

Non-limiting examples of silicone carboxylates include those commercially available from Noveon under the name Ultrasil® CA-1 Silicone (Dimethicone PEG-7 Phthalate) and Ultrasil® CA-2 Silicone (Dimethicone PEG-7 Succinate), both of which correspond to formula (XV) below. Thus, in one embodiment, the at least one silicone carboxylate is chosen from a compound of formula (XV) and salts thereof:

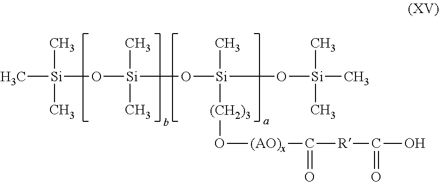

wherein a is an integer ranging from 1 to 100, b is an integer ranging from 0 to 500, AO is chosen from groups of the following formula:

-(EO)c-(PO)d-(EO)e-

wherein c, d, and e, which may be identical or different, are each integers ranging from 0 to 20; EO is an ethylene oxide group; PO is a propylene oxide group; x is an integer ranging from 0 to 60; R′ is chosen from optionally substituted divalent hydrocarbons, such as alkylene groups and alkenylene groups comprising from 2 to 22 carbon atoms, and optionally substituted divalent aromatic groups, such as groups of the following formula:

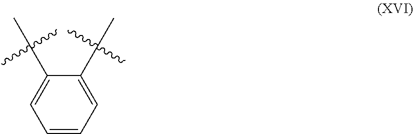 [SMILES: *c1ccccc1*]

and groups of formula

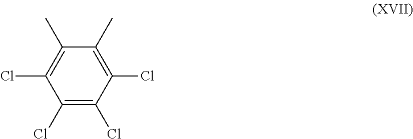

Non-limiting examples of silicone carboxylates include those described in U.S. Pat. Nos. 5,248,783 and 5,739,371, the disclosures of which are incorporated herein by reference, and which are silicone compounds of formula (XIII).

The carboxysilicone will typically be present in the composition of the present disclosure in an amount of from about 0.01% to about 20%, by weight, in some particular embodiments from about 0.5% to about 10%, by weight, and in some particular embodiments from about 0.5% to about 5.0%, by weight, including all ranges and subranges therebetween, based on the total weight of the composition.

In accordance with the various embodiments, the carboxysilicone compound is present in the compositions according to the disclosure in amounts range from about 0.01% to about 20%, and in some embodiments from about 0.1% to about 15%, and in some further embodiments from about 0.1% to about 10%. In some representative embodiments, the carboxysilicone compound is present from about 0.1% to about 5%, and from about 0.2% to about 3%. In yet other embodiments, the carboxysilicone compound is present from at least 0.2%. And in yet other embodiments, the carboxysilicone compound is present from at least 0.2% to up to about 40%. In some specific embodiments, the carboxysilicone compound is present at about 1.5%. In some specific embodiments, the carboxysilicone compound is present at about 0.75%. In some other specific embodiments, the carboxysilicone compound is present at about 0.25%.

Thus, the carboxysilicone compound may be present from about 0.01, 0.05, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, to about 20 percent by weight, including increments and ranges therein and there between.

Solvent

The composition, according to the present disclosure, further includes suitable solvents for treatment of keratinous fibers. Examples of suitable solvents include water, in some particular embodiments distilled or de-ionised, or organic solvents as a carrier and solvent for the polycarbodiimides and carboxysilicone polymers.

Suitable organic solvents may be chosen from volatile and nonvolatile organic solvents.

Suitable organic solvents are typically C1-C4 lower alcohols, glycols, polyols, polyol ethers, hydrocarbons, and oils. Examples of organic solvents include, but are not limited to, ethanol, isopropyl alcohol, benzyl alcohol, phenyl ethyl alcohol, propylene glycol, pentylene glycol, hexylene glycol, glycerol, and mixtures thereof.

Other suitable organic solvents include glycol ethers, for example, ethylene glycol and its ethers such as ethylene glycol monomethyl ether, ethylene glycol monopropyl ether, ethylene glycol monobutyl ether, propylene glycol and its ethers, such as propylene glycol monomethyl ether, propylene glycol monopropyl ether, propylene glycol monobutyl ether, dipropylene glycol and diethylene glycol alkyl ethers, such as diethylene glycol monoethyl ether, diethylene glycolmonobutyl ether, and dipropylene glycol n-butyl ether. Glycol ethers are commercially available from The Dow Chemical Company under the DOW E-series and DOW P-series. In an exemplary embodiment, glycol ether for use in the present invention is dipropylene glycol n-butyl ether, known under the tradename of DOWANOL DPnB.

Suitable organic solvents also include synthetic oils and hydrocarbon oils include mineral oil, petrolatum, and C10-C40 hydrocarbons which may be aliphatic (with a straight, branched or cyclic chain), aromatic, arylaliphatic such as paraffins, iso-paraffins, isododecanes, aromatic hydrocarbons, polybutene, hydrogenated polyisobutene, hydrogenated polydecene, polydecene, squalene, petrolatum and isoparaffins, silicone oils, fluoro oils and mixtures, thereof.

The term “hydrocarbon based oil” or “hydrocarbon oil” refers to oil mainly containing hydrogen and carbon atoms and possibly oxygen, nitrogen, sulfur and/or phosphorus atoms. Representative examples of hydrocarbon based oils include oils containing from 8 to 16 carbon atoms, and especially branched C8 C16 alkanes (also known as isoparaffins), for instance isododecane (also known as 2,2,4,4,6 pentamethylheptane), isodecane and isohexadecane.

Examples of silicone oils that may be useful in the present invention include nonvolatile silicone oils such as polydimethylsiloxanes (PDMS), polydimethylsiloxanes comprising alkyl or alkoxy groups that are pendent and/or at the end of a silicone chain, these groups each containing from 2 to 24 carbon atoms, phenyl silicones, for instance phenyl trimethicones, phenyl dimethicones, phenyl trimethylsiloxy diphenylsiloxanes, diphenyl dimethicones, diphenyl methyldiphenyl trisiloxanes and 2 phenylethyl trimethylsiloxysilicates, and dimethicones or phenyltrimethicones with a viscosity of less than or equal to 100 cSt.

Other representative examples of silicone oils that may be useful in the present invention include volatile silicone oils such as linear or cyclic silicone oils, and especially containing from 2 to 10 silicon atoms and in particular from 2 to 7 silicon atoms, these silicones optionally comprising alkyl or alkoxy groups containing from 1 to 10 carbon atoms. Specific examples include dimethicones with a viscosity of 5 and 6 cSt, octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane and dodecamethylpentasiloxane, and mixtures thereof. Representative examples of fluoro oils that may be suitable for use in the present invention include volatile fluoro oils such as nonafluoromethoxybutane and perfluoro methylcyclopentane.

Particularly suitable solvents in a composition of the present disclosure include water, isododecane, ethanol, and combinations thereof. The solvent will typically be present in total amounts ranging from about 60% to 98%, in some embodiments, from 80% to 96%, by weight, including all ranges and subranges therebetween, based on the total weight of the composition. Thus, solvents may be present in amounts of about 98%, 97%, 96%, 95%, 94%, 93%, 92%, 91%, 90%, 85%, 80%, 75%, 70%, 65%, 60%, and about 55%, 50%, 48%, 46%, 45%, 44%, 42%,40%, by weight, based on the total weight of the composition, including increments and ranges therein and there between from about 40% to about 98%.

In yet some other embodiments, the solvent of the present dislcosure does not comprise water and/or organic solvent that is added as a separate ingredient, by itself, into the compositions of the present invention, such that water and/or organic solvent is present in the compositions of the present invention when it accompanies one or more ingredients of a raw material that is added into the compositions of the invention.

When the compositions of the disclosure contain water, according to various embodiments, water can be present in amounts of about 98% or less, such as about 96%, 95%, 90%, 85%, 80%, 75%, 70%, 65%, 60%, 55%, 50%, 48%, 46%, 45%, 44%, 42%,40%, 35%,30%, 20%, 10%, or 5% or less, by weight, based on the total weight of the composition, including increments and ranges therein and there between.

When the compositions of the disclosure contain an organic solvent(s), according to various embodiments, the organic solvent(s) can be present in a total amount of about 98% or less, such as about 96%, 95%, 90%, 85%, 80%, 75%, 70%, 65%, 60%, 55%, 50%, 48%, 46%, 45%, 44%, 42%,40%, 35%,30%, 20%, 10%, or 5% or less, by weight, based on the total weight of the composition, including increments and ranges therein and there between.

Additives

The composition, according to the present disclosure, further includes suitable additives for treatment of keratinous fibers.

The composition according to the disclosure may also comprise additives chosen from amine or amino compounds (e.g., amino silicones, polyamines, diamines, alkyl monoamines, alkoxylated monoamines, alkoxylated polyaminesand amino functionalized silane compounds), surfactants (anionic, nonionic, cationic and amphoteric/zwtterionic), and polymers other than the polycarbodiimide and caroxysilicone polymers of the invention such as anionic polymers, nonionic polymers, amphoteric polymers, polymeric rheology modifiers, thickening and/or viscosity modifying agents, associative or non-associative polymeric thickeners. Other suitable additives may be chosen from non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof.

The compositions of certain embodiments may comprise stabilizers, for example sodium chloride, magnesium dichloride or magnesium sulfate.

The amine or amino compounds that may be employed in the compositions of the present disclosure may include amino silicones. The term “amino” is intended to mean refer to a primary, secondary or tertiary amine or a quaternary ammonium group

The amino silicones that may be employed in the compositions of the present disclosure may be chosen from polysiloxanes having at least one primary, secondary or tertiary amine group such as trimethylsilylamodimethicones, quaternary ammonium silicones, multiblock polyoxyalkylenated amino silicones, of type (AB)n, A being a polysiloxane block and B being a polyoxyalkylenated block containing at least one amine group, alkyl amino silicones, and mixtures thereof.

Suitable examples of the amine or amino compounds include amodimethicone (e.g., sold under the name KF 8020 by Shin Etsu or XIAMETER® MEM-8299 Cationic Emulsion by Dow Corning), and bis-cetearyl amodimethicone (sold under the name SILSOFT AX by Momentive).

The amine or amino compounds that may be employed in the compositions of the present disclosure may also be chosen from alkyl monoamines, alkoxylated polyamines, alkoxylated monoamines, and polyamines, in particular, those that do not contain silicon atoms or silicone moieties.

Suitable examples of alkyl monoamines include, but are not limited to the following examples: dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

The alkyl monoamines may also be chosen from amidoamines, including, but not limited to the following examples: oleamidopropyl dimethylamine, stearamidopropyl dimethylamine, isostearamidopropyl dimethylamine, stearamidoethyl dimethylamine, lauramidopropyl dimethylamine, palmitamidopropyl dimethylamine, stearamidoethyldiethylamine, and mixtures thereof.

The alkoxylated polyamines that may be employed in the compositions of the present disclosure are chosen from amino compounds having at least two amino groups and at least one degree of alkoxylation provided by an alkylene oxide group which is in certain embodiments chosen from ethylene oxide and propylene oxide.

Suitable examples of alkoxylated polyamines include, but are not limited to diamine and triamine compounds belonging to the JEFFAMINE series such as the JEFFAMINE D, JEFFAMINE ED, JEFFAMINE EDR, and JEFFAMINE T series available from Huntsman Corporation.

The alkoxylated monoamines that may be employed in the compositions of the present disclosure are chosen from amino compounds having at one amino groups and at least one degree of alkoxylation provided by an alkylene oxide group which is in certain embodiments chosen from ethylene oxide and propylene oxide.

Suitable examples are alkoxylated derivatives of cocamine, lauramine, palmitamine, rapeseedamine, oleamine, soyamine, stearamine, tallow amine, tallow aminopropylamine, behenyl propylenediamine and those of the JEFFAMINE M series from Huntsman.

The polyamines that may be used in the compositions of the present disclosure may in particular be chosen from aminosilicones, polyvinylamines, aminated polysaccharides, amine substituted polyalkylene glycols, amine substituted polyacrylate crosspolymers, amine substituted polyacrylates, amine substituted polymethacrylates, proteins, protein derivatives, amine substituted polyesters, polyamino acids, polyalkylamines, diethylene triamine, triethylenetetramine, spermidine, spermine and mixtures thereof.

The rheology modifiers and thickening/viscosity-modifying agents that may be employed in compositions of the present disclosure may include any water-soluble or water-dispersible compound that is compatible with the polycarbodiimide, carboxysilicone polymers, and compositions of the disclosure, such as acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.

Other Additives

Cationic Polymers containing at least One Carboxyl Group.

The cationic polymer can have a negative charge but remains cationic overall, can be an amphoteric polymer that can carry a cationic charge based on pH, or can be a betaine polymer that remains amphoteric at any pH.

The cationic polymers are polymers that result from the homopolymerization or copolymerization of ethylenically unsaturated monomers chosen from: (i) at least one nonionic monomer such as (Alkyl)(Meth)Acrylamide, (Alkyl)(Meth)Acrylate Ester, Vinyl Pyrrolidone, Vinyl Imidazole; (ii) at least one cationic monomer such as Ethyltrimonium (Alkyl)(Meth)Acrylamide, Ethyltrimonium (Alkyl)(Meth)Acrylate Ester, Vinylimidazoline, Dimethylaminopropyl (Alkyl)(Meth)Acrylamide, Methacrylamidopropyl Triethyl Ammonium Chloride (MAPTAC), Diallyl Dimethyl Ammonium Chloride (DADMAC); (iii) at least one (Alkyl)Acrylic acid; (iv) at least one amphoteric monomer such as a carboxybetaine zwitterionic monomer.

Suitable examples of such cationic polymers are: the diallyidimethylammonium chloride/acrylic acid copolymers sold under the names MERQUAT 280 POLYMER or MERQUAT 280NP POLYMER or MERQUAT 281 POLYMER or MERQUAT 295 POLYMER, by the company Nalco (Lubrizol) (INCI name: Polyquaternium-22); the copolymer of methacrylamidopropyltrimonium chloride, of acrylic acid and or methyl acrylate, sold under the name MERQUAT 2001 POLYMER OR MERQUAT 2001N POLYMER by the company Nalco (Lubrizol) (INCI name: Polyquaternium-47); the acrylamide/dimethyldiallylammonium chloride/acrylic acid terpolymer sold under the name MERQUAT 3330DRY POLYMER or MERQUAT 3330PR POLYMER or MERQUAT 3331PR POLYMER or MERQUAT 3940 POLYMER or MERQUAT PLUS 3330 POLYMER OR MERQUAT PLUS 3331 POLYMER by the company Nalco (Lubrizol) (INCI name:: Polyquaternium-39); an ampholytic terpolymer consisting of methacrylamidopropyl trimethyl ammonium chloride (MAPTAC), acrylamide and acrylic acid, sold under the name MERQUAT 2003PR POLYMER by the company Nalco (Lubrizol) (INCI name:: Polyquaternium-53); Polyquaternium-30, Polyquaternium-35, Polyquaternium-45, Polyquaternium-50, Polyquaternium-54; Polyquaternium-57; Polyquaternium-63; Polyquaternium-74; Polyquaternium-76; Polyquaternium-86; Polyquaternium-89; Polyquaternium-95; Polyquaternium-98, Polyquaternium-104; Polyquaternium-111; Polyquaternium-112, and mixtures thereof.

Polycarboxylic Acid Polymer Compounds

The polycarboxylic acid, compounds of the present disclosure may be chosen from anionic, nonionic, and amphoteric polymers.

The anionic polymers may be soluble in a cosmetically acceptable medium or insoluble in this same medium such that they may be used in the form of dispersions of solid or liquid particles of polymer (latex or pseudolatex).

The anionic polymers may be selected from polymers comprising groups derived from carboxylic acids, and have an average molecular weight by number of between about 500 and 5,000,000. The carboxylic groups are provided by unsaturated mono- or diacid carboxylic monomers such as those that have the formula:

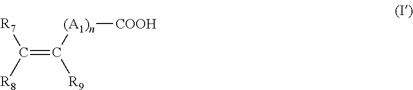

in which n is an integer from 0 to 10, A₁ denotes a methylene group optionally joined to the carbon atom of the unsaturated group or to the adjacent methylene group when n is greater than 1, via a heteroatom such as oxygen or sulfur, R₇ denotes a hydrogen atom or a phenyl or benzyl group, R₈ denotes a hydrogen atom or a lower alkyl or carboxyl group, and R₉ denotes a hydrogen atom, a lower alkyl group, a CH₂—COOH, phenyl or benzyl group.

In the abovementioned formula, a lower alkyl group in certain embodiments denotes a group containing 1 to 4 carbon atoms and in particular methyl and ethyl groups.

The polycarboxylic acid compounds include Copolymers of acrylic or methacrylic acid or salts thereof, and in particular copolymers of acrylic acid and acrylamide sold in the form of their sodium salts.

The polycarboxylic acid compounds include Copolymers of acrylic or methacrylic acid with a monoethylenic monomer such as ethylene, styrene, vinyl esters and acrylic or methacrylic acid esters, optionally grafted onto a polyalkylene glycol such as polyethylene glycol and optionally crosslinked. The polycarboxylic acid compounds include methacrylic acid/acrylic acid/ethyl acrylate/methyl methacrylate copolymers in an aqueous dispersion.

The polycarboxylic acid compounds include Crotonic acid copolymers, such as those comprising vinyl acetate or propionate units in their chain and optionally other monomers such as allyl esters or methallyl esters, vinyl ether or vinyl ester of a linear or branched saturated carboxylic acid with a long hydrocarbon-based chain, such as those containing at least 5 carbon atoms, it being possible for these polymers optionally to be grafted or crosslinked, or alternatively another vinyl, allyl or methallyl ester monomer of an [alpha]- or [beta]-cyclic carboxylic acid.

The polycarboxylic acid compounds include Copolymers of C4-C8 monounsaturated carboxylic acids selected from: copolymers comprising (i) one or more maleic, fumaric, itaconic, allyloxyacetic, methallyloxyacetic, 3-allyloxypropionic, allylthioacetic, allylaminoacetic, vinylacetic, vinyloxyacetic, crotyloxyacetic, 3-butenoic, 4-pentenoic, 10-undecenoic, allylmalonic, maleamic, itaconamic, N-monohydroxyalkyl- or N-dihydroxy-alkyl-maleamic acids and (ii) at least one monomer selected from vinyl esters, vinyl ethers, vinyl halides, phenylvinyl derivatives, acrylic acid and its esters, the anhydride functions of these copolymers optionally being monoesterified or monoamidated. The polycarboxylic acid compounds include copolymers comprising (i) one or more maleic, citraconic or itaconic anhydride units and (ii) one or more monomers selected from allyl or methallyl esters optionally comprising one or more acrylamide, methacrylamide, [alpha]-olefin, acrylic or methacrylic ester, acrylic or methacrylic acid or vinylpyrrolidone groups in their chain, the anhydride functions of these copolymers optionally being monoesterified or monoamidated. The polycarboxylic acid compounds include polyacrylamides comprising carboxylate groups.

The polycarboxylic acid compounds of the present disclosure may also include those anionic polymers as sold under the FIXATE series as commercially available from Lubrizol, such as a branched block anionic polymer sold as FIXATE G-100, a branched anionic acrylate copolymer Polyacrylate-2 Crosspolymer (FIXATE SUPERHOLD polymer), Acrylates Crosspolymer-3 (FIXATE FREESTYLE Polymer), Polyacrylate-14 (FIXATE PLUS Polymer), those sold under the CARBOPOL series as commercially available from Lubrizol such as Acrylates Crosspolymer-4 (CARBOPOL AQUA SF-2), Acrylates Crosspolymer-4 (CARBOPOL AQUA CC), and those sold under the SYNTRAN series as commercially available from Interpolymer such as Acrylates Copolymer (SYNTRAN 5190), Styrene/Acrylates/Ammonium Methacrylate Copolymer (SYNTRAN 5760), and Ammonium Acrylates Copolymer (SYNTRAN KL-219C).

The polycarboxylic acid compounds of the present disclosure also includes anionic latex polymers such as acrylic copolymer and (meth)acrylate copolymers dispersions.

The polycarboxylic acid compounds include copolymers of acrylic acid or of acrylic esters, such as Acrylates/t-Butylacrylamide copolymer sold as ULTRAHOLD 8, acrylic acid/ethyl acrylate/N-tert-butylacrylamide terpolymers sold especially as ULTRAHOLD STRONG by BASF, copolymers derived from crotonic acid, such as vinyl acetate/vinyl tert-butylbenzoate/crotonic acid terpolymers and the crotonic acid/vinyl acetate/vinyl neododecanoate terpolymers sold especially as RESYN 28-29-30 by Azko Nobel, polymers derived from maleic, fumaric or itaconic acids or anhydrides with vinyl esters, vinyl ethers, vinyl halides, phenylvinyl derivatives and acrylic acid and esters thereof, such as the methyl vinyl ether/monoesterified maleic anhydride copolymers sold, for example, as GANTREZ AN or ES by ISP, the copolymers of methacrylic acid and methyl methacrylate sold as EUDRAGIT L by Rohm Pharma, the copolymers of methacrylic acid and ethyl acrylate sold as LUVIMER MAEX or MAE by BASF, the vinyl acetate/crotonic acid copolymers sold as LUVISET CA 66 by BASF, the vinyl acetate/crotonic acid copolymers grafted with polyethylene glycol sold as ARISTOFLEX A by BASF, and the polymer sold as FIXATE G-100 by Noveon.

The polycarboxylic acid compounds include amphoteric polymers which may be selected from the following polymers:

copolymers having acidic vinyl units and basic vinyl units, such as those resulting from the copolymerization of a monomer derived from a vinyl compound bearing a carboxylic group such as, more particularly, acrylic acid, methacrylic acid, maleic acid, alpha-chloroacrylic acid, and a basic monomer derived from a substituted vinyl compound containing at least one basic atom, such as, more particularly, dialkylaminoalkyl methacrylate and acrylate, dialkylaminoalkylmethacrylamides and acrylamides. Such compounds are described in U.S. Pat. No. 3,836,537.

The polycarboxylic acid compounds include Polymers comprising units derived from:

at least one monomer selected from acrylamides and methacrylamides substituted on the nitrogen atom with an alkyl group,

at least one acidic comonomer containing one or more reactive carboxylic groups, selected more particularly from acrylic, methacrylic, crotonic, itaconic, maleic and fumaric acid and alkyl monoesters, having 1 to 4 carbon atoms, of maleic or fumaric acid or anhydride, and

at least one basic comonomer such as esters with primary, secondary, tertiary or quaternary amine substituents of acrylic and methacrylic acids and the product of quaternization of dimethylaminoethyl methacrylate with dimethyl or diethyl sulfate. The N-substituted acrylamides or methacrylamides that are more particularly preferred according to the invention are compounds in which the alkyl groups contain from 2 to 12 carbon atoms and more particularly N-ethylacrylamide, N-tert-butylacrylamide, N-tert-octylacrylamide, N-octylacrylamide, N-decylacrylamide, N-dodecylacrylamide and the corresponding methacrylamides. The preferred basic comonomers are aminoethyl, butylaminoethyl, N,N′-dimethylaminoethyl and N-tert-butylaminoethyl methacrylates.

The copolymers whose CTFA (4th edition, 1991) name is octylacrylamide/acrylates/butylaminoethyl methacrylate copolymer, such as the products sold as AMPHOMER LV 71, Acrylates/octylacrylamide copolymer sold as Amphomer 28-4961 or LOVOCRYL 47 by National Starch, are particularly used.

The polycarboxylic acid compounds include Crosslinked and acylated polyaminoamides.

The polycarboxylic acid compounds include carboxylic acids selected from acids having 6 to 10 carbon atoms, such as adipic acid, 2,2,4-trimethyladipic acid and 2,4,4-trimethyladipic acid, terephthalic acid, acids containing an ethylenic double bond such as, for example, acrylic acid, methacrylic acid and itaconic acid.

The polycarboxylic acid compounds include Polymers comprising zwitterionic units of formula:

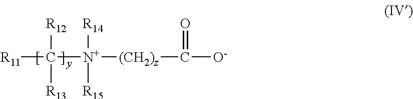

in which R11 denotes a polymerizable unsaturated group such as an acrylate, methacrylate, acrylamide or methacrylamide group, y and z represent an integer from 1 to 3, R12 and R13 represent a hydrogen atom, a methyl, ethyl or propyl group, R14 and R15 represent a hydrogen atom or an alkyl group such that the sum of the carbon atoms in R14 and R15 does not exceed 10.

The polymers comprising such units may also comprise units derived from non-zwitterionic monomers such as dimethyl- or diethylaminoethyl acrylate or methacrylate or alkyl acrylates or methacrylates, acrylamides or methacrylamides or vinyl acetate, for example, methyl methacrylate/methyl dimethylcarboxymethylammonioethyl methacrylate copolymers such sold as DIAFORMER Z301 by Sandoz;The polycarboxylic acid compounds include Polymers derived from chitosan comprising monomer units corresponding to the following formulae:

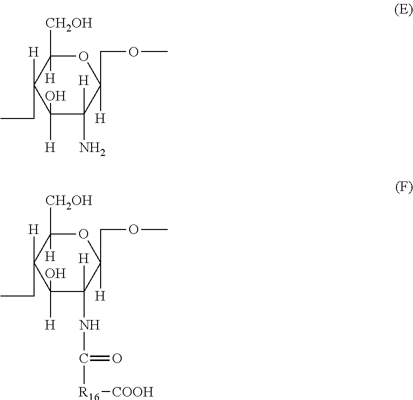

the unit (D) being present in proportions of between 0 and 30 percent, the unit (E) in proportions of between 5 percent and 50 percent and the unit (F) in proportions of between 30 percent and 90 percent, it being understood that, in this unit (F), R16 represents a group of formula:

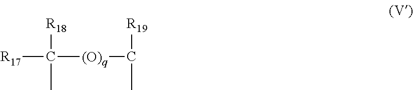

in which, if q=0, R17, R18 and R19, which may be identical or different, each represent a hydrogen atom, a methyl, hydroxyl, acetoxy or amino residue, a monoalkylamine residue or a dialkylamine residue that are optionally interspersed with one or more nitrogen atoms and/or optionally substituted with one or more amine, hydroxyl, carboxyl, alkylthio or sulfonic groups, an alkylthio residue in which the alkyl group bears an amino residue, at least one of the groups R17, R18 and R19 being, in this case, a hydrogen atom;

or, if q=1, R17, R18 and R19 each represent a hydrogen atom, and also the salts formed by these compounds with bases or acids.

The polycarboxylic acid compounds include Polymers with units corresponding to the general formula (VI') are described, for example, in French patent 1 400 366:

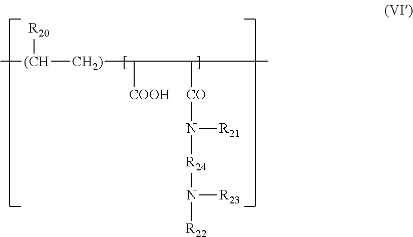

in which R20 represents a hydrogen atom, a CH3O, CH3CH2O or phenyl group, R21 denotes a hydrogen atom or a lower alkyl group such as methyl or ethyl, R22 denotes a hydrogen atom or a C1-C6 lower alkyl group such as methyl or ethyl, R23 denotes a C1-C6 lower alkyl group such as methyl or ethyl or a group corresponding to the formula: —R24-N(R22)2, R24 representing a group —CH2-CH2-, —CH2-CH2-CH2- or —CH2-CH(CH3)-, R22 having the meanings mentioned above.

The polycarboxylic acid compounds include Polymers derived from the N-carboxyalkylation of chitosan, such as N-carboxymethylchitosan or N-carboxybutylchitosan.

The polycarboxylic acid compounds include Amphoteric polymers of the type -D-X-D-X selected from:

a) Polymers obtained by the action of chloroacetic acid or sodium chloroacetate on compounds comprising at least one unit of formula:

-D-X-D-X-D- (VII′) where D denotes a

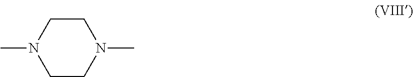

group and X denotes the symbol E or E′; E or E′ may be identical or different and denote a divalent group that is an alkylene group with a straight or branched chain containing up to 7 carbon atoms in the main chain, which is unsubstituted or substituted by hydroxyl groups and which may comprise, in addition to oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) Polymers having the formula:

-D-X-D-X- (X′) where D denotes a

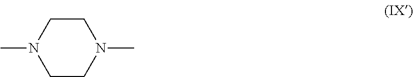

group and X denotes the symbol E or E′ and at least once E′; E having the meaning given above and E′ is a divalent group that is an alkylene group with a straight or branched chain having up to 7 carbon atoms in the main chain, which is unsubstituted or substituted with one or more hydroxyl groups and containing one or more nitrogen atoms, the nitrogen atom being substituted with an alkyl chain that is optionally interspersed by an oxygen atom and necessarily comprising one or more carboxyl functions or one or more hydroxyl functions and betainized by reaction with chloroacetic acid or sodium chloroacetate.

The polycarboxylic acid compounds include (C1-C5)Alkyl vinyl ether/maleic anhydride copolymers partially modified by semiamidation with an N,N-dialkylaminoalkylamine such as N,N-dimethylaminopropylamine or by semiesterification with an N,N-dialkylaminoalkanol. These copolymers may also comprise other vinyl comonomers such as vinylcaprolactam.

Among the amphoteric polymers described above, the ones that are most preferred are Octylacrylamide/acrylates/butylamino ethyl methacrylate copolymer, such as the products sold as AMPHOMER, AMPHOMER LV 71 or LOVOCRYL 47 by National Starch and the copolymers of methyl methacrylate/methyl dimethylcarboxy-methylammonioethyl methacrylate, sold, for example, as DIAFORMER Z301 by Sandoz.

The polycarboxylic acids of the present disclosure may be chosen from compounds which are known to be used as rheology modifiers or thickeners in cosmetic compositions. Such polymers include anionic and amphoteric polymers, for example crosslinked homopolymers of acrylic acid, associative polymers, non-associative thickening polymers, and water-soluble thickening polymers. Such polymers may also be chosen from nonionic, anionic, cationic and amphoteric amphiphilic polymers. The rheology modifiers or thickeners that can used may include those polycarboxylic acid compounds described above.

The amphiphilic polymers may, optionally, contain a hydrophobic chain that is a saturated or unsaturated, aromatic or non-aromatic, linear or branched C6-C30 hydrocarbon-based chain, optionally comprising one or more oxyalkylene (oxyethylene and/or oxypropylene) units.

Representative examples of such amphiphilic polymers are:

nonionic amphiphilic polymers containing a hydrophobic chain such as:

copolymers of C1-C6 alkyl(meth)acrylates and of amphiphilic monomers containing a hydrophobic chain;

copolymers of hydrophilic (meth)acrylates and of hydrophobic monomers containing at least one hydrophobic chain, for instance the polyethylene glycol methacrylate/lauryl methacrylate copolymer;

anionic amphiphilic polymers containing at least one hydrophobic chain which are crosslinked or non-crosslinked, contain at least one hydrophilic unit derived from one or more ethylenically unsaturated monomers bearing a carboxylic acid function, which is free or partially or totally neutralized, and at least one hydrophobic unit derived from one or more ethylenically unsaturated monomers bearing a hydrophobic side chain, and optionally at least one crosslinking unit derived from one or more polyunsaturated monomers.

Examples of anionic amphiphilic polymers include CARBOPOL ETD-2020 (acrylic acid/C10-C30 alkyl methacrylate crosslinked copolymer sold by the company Noveon); CARBOPOL 1382, PEMULEN TR1 and PEMULEN TR2 (acrylic acid/C10-C30 alkyl acrylatecrosslinked copolymers-sold by the company Noveon), the methacrylic acid/ethyl acrylate/oxyethylenated stearyl methacrylate copolymer (55/35/10); the (meth)acrylic acid/ethyl acrylate/25 EO oxyethylenated behenyl methacrylate copolymer (ACULYN 28 sold by Rohm and Haas) and the methacrylic acid/ethyl acrylate/steareth-10 allyl ether crosslinked copolymer.

Other examples include cross-linked acrylic polymers, for example those sold under the CARBOPOL SF series, such as ethyl acrylate/methacrylic acid copolymer with INCI name: acrylates copolymer, sold under the name CARBOPOL SF1(R) by the LUBRIZOL company.

Yet other examples include anionic polymers also known as anionic thickening polymers chosen from carbomers, acrylate copolymers, and crosslinked terpolymers of methacrylic acid, ethylacrylate, and polyethylene glycol (10 EO) stearyl alcohol ether (Steareth 10), such as the products sold by the company ALLIED COLLOIDS under the names SALCARE SC 80 and SALCARE SC 90, which are aqueous emulsions containing 30 percent of a crosslinked terpolymer of methacrylic acid, of ethyl acrylate and of steareth-10-allyl ether (40/50/10).

Anionic thickening polymers can also be chosen from:

terpolymers formed from maleic anhydride/C30-C38 alpha-olefin/alkyl maleate such as the product (maleic anhydride/C30-C38 alpha-olefin/isopropyl maleate copolymer) sold under the name PERFORMA 1608 by the company NEWPHASE TECHNOLOGIES;

acrylic terpolymers formed from: (a) 20 percent to 70 percent by weight of a carboxylic acid with alpha, beta-monoethylenic unsaturation; (b) 20 percent to 80 percent by weight of a nonsurfactant monomer with alpha, beta-monoethylenic unsaturation different from (a); and (c) 0.5 percent to 60 percent by weight of a nonionic monourethane which is the product of the reaction of a monohydric surfactant with a monoisocyanate with monoethylenic unsaturation; (3) copolymers formed from at least two monomers, wherein at least one of the two monomers is chosen from a carboxylic acid with alpha, beta-monoethylenic unsaturation, an ester of a carboxylic acid with alpha, beta-monoethylenic unsaturation, and an oxyalkylenated fatty alcohol; and (4) copolymers formed from at least three monomers, wherein at least one of the three monomers is chosen from a carboxylic acid with alpha, beta-monoethylenic unsaturation, at least one of the three monomers is chosen from an ester of a carboxylic acid with alpha beta-monoethylenic unsaturation and at least one of the three monomers is chosen from an oxyalkylenated fatty alcohol.

Additionally, these compounds can also contain, as a monomer, a carboxylic acid ester comprising an apha, beta-monoethylenic unsaturation and a C1-C4 alcohol. By way of example of this type of compound, there may be mentioned ACULYN 22 sold by the company ROHM and HAAS, which is an oxyalkylenated stearyl methacrylate/ethylacrylate/methacrylic acid terpolymer.

The polycarboxylic acid compounds include associative polyurethanes, associative unsaturated polyacids, and associative polymers or copolymers containing at least one monomer comprising ethylenic unsaturation.

A representative example of an associative polyurethane is methacrylic acid/methyl acrylate/ethoxylated (40 EO) behenyl alcohol dimethyl(meta-isopropenyl)benzyl isocyanate terpolymer as a 25 percent aqueous dispersion, known by the trade name, VISCOPHOBE DB 1000 and commercially available from Amerchol.

According to some embodiments of the present invention, the polycarboxylic acid compounds include at least one acrylic acid-based, (meth)acrylic acid-based, acrylate-based or (meth)acrylate-based monomer having anionic and/or cationic functionalities. Suitable compounds include, but are not limited to, polymers comprising polyacrylates such as those identified in the International Cosmetic Ingredient Dictionary and Handbook (9 th ed. 2002) such as, for example, polyacrylate-1, polyacrylate-2, polyacrylate-3, polyacrylate-4, polyacrylate-16, polyacrylate-17, polyacrylate-18, polyacrylate-19, polyacrylate-21, and mixtures thereof. Such (co)polymers, or similar (co)polymers, can be combined individually or with other (co)polymers in such a way to form suitable bimodal agents having both cationic and anionic functionalities. According to certain embodiments, the bimodal agent is selected from the group consisting of polymers consisting of polyacrylate-21 and acrylates/dimethylaminoethylmethacrylate copolymer (marketed under the name SYNTRAN PC 5100 by Interpolymer), polyacrylate-16 (marketed under the name SYNTRAN PC 5112 by Interpolymer), and polyacrylate-18 and polyacrylate-19 (marketed under the names SYNTRAN PC 5107 or SYNTRAN PC 5117 b_{y} Interpolymer).

Latex Polymers

According to various exemplary embodiments, the compositions of the present invention can further comprise one or more latex polymers (also referred to as “latex polymers” in this application) which are different from the carboxysilicone polymers of the present invention and can be chosen from carboxyl functional acrylate latex polymers, carboxyl functional polyurethane latex polymers, carboxyl functional silicone latex polymers, carboxyl functional non-acrylate latex polymers and mixtures thereof.

In various embodiments, the latex polymers of the present invention can be film-forming latex polymers or non film-forming latex polymers.

In at least certain embodiments of the disclosure, the latex polymers are provided in the form of aqueous dispersions prior to formulating the compositions of the disclosure. In various embodiments, the aqueous dispersions may be obtained through an emulsion polymerization of monomers wherein the resulting latex polymers have a particle size lower than about 1 micron. In at least one exemplary embodiment, a dispersion prepared by the polymerization in water of one or more monomers having a polymerizable double bond may be chosen. In another exemplary embodiment, the aqueous dispersions obtained through an emulsion polymerization may be spray-dried.

In other embodiments, the latex polymers are produced from condensation reactions between monomers and subsequently dispersed in an aqueous medium.

Thus, the latex polymers may, in various exemplary embodiments, exist as dispersed polymer particles in a dispersion medium, such as an aqueous dispersion medium. The latex polymers may, in various embodiments, each be dispersed in independent dispersion media or dispersed together in the same dispersion medium.

The dispersion medium comprises at least one solvent chosen from water. The dispersion medium may further comprise at least one solvent chosen from cosmetically acceptable organic solvents such as those described above.

In embodiments according to the disclosure, the latex polymer particles are not soluble in the solvent of the dispersion medium, i.e. are not water soluble and/or are not soluble in the at least one cosmetically acceptable organic solvent. Accordingly, the latex polymers retain their particulate form in the solvent or solvents chosen.

In at least certain exemplary embodiments, latex polymer particles according to the disclosure may have an average diameter ranging up to about 1000 nm, such as from about 50 nm to about 800 nm, or from about 100 nm to about 500 nm. Such particle sizes may be measured with a laser granulometer (e.g. Brookhaven B190).

In various embodiments, the latex polymers may, independently, be neutralized, partially neutralized, or unneutralized. In exemplary embodiments where the latex polymers are neutralized or partially neutralized, the particle size may be, for example, greater than about 800 nm. In at least certain embodiments, the particulate form of the latex polymers is retained in the dispersion medium.

In further embodiments, the latex polymers may be chosen from uncharged and charged latex polymers. Thus, the latex polymers may, according to various exemplary embodiments, be chosen from nonionic latex polymers, cationic latex polymers, anionic latex polymers and amphoteric latex polymers.

By way of non-limiting example only, the latex polymers may be chosen from carboxyl functional acrylate latex polymers, such as those resulting from the homopolymerization or copolymerization of ethylenically unsaturated monomers chosen from vinyl monomers, (meth)acrylic monomers, (meth)acrylamide monomers, mono- and dicarboxylic unsaturated acids, esters of (meth)acrylic monomers, and amides of (meth)acrylic monomers The term “(meth)acryl” and variations thereof, as used herein, means acryl or methacryl.

The (meth)acrylic monomers may be chosen from, for example, acrylic acid, methacrylic acid, citraconic acid, itaconic acid, maleic acid, fumaric acid, crotonic acid, and maleic anhydride. The esters of (meth)acrylic monomers may be, by way of non-limiting example, C1-C8 alkyl (meth)acrylates such as methyl (meth)acrylate, ethyl (meth)acrylate, propyl (meth)acrylate, isopropyl (meth)acrylate, butyl (meth)acrylate, tert-butyl (meth)acrylate, pentyl(meth) acrylate, isopentyl (meth)acrylate, neopentyl (meth)acrylate, hexyl (meth)acrylate, isohexyl (meth)acrylate, 2-ethylhexyl (meth)acrylate, cyclohexyl (meth)acrylate, isohexyl (meth)acrylate, heptyl (meth)acrylate, isoheptyl (meth)acrylate, octyl (meth)acrylate, isooctyl (meth)acrylate, allyl (meth)acrylate, and combinations thereof. The amides of (meth)acrylic monomers can, for example, be made of (meth)acrylamides, and especially N-alkyl (meth)acrylamides, in particular N—(C1-C12) alkyl (meth)acrylates such as N-ethyl (meth)acrylamide, N-t-butyl (meth)acrylamide, N-t-octyl (meth)acrylamide, N-methylol (meth)acrylamide and N-diacetone (meth)acrylamide, and any combination thereof.

The vinyl monomers can include, but are not limited to, vinyl cyanide compounds such as acrylonitrile and methacrylonitrile; vinyl esters such as vinyl formate, vinyl acetate, vinyl propionate, vinyl neodecanoate, vinyl pivalate, vinyl benzoate and vinyl t-butyl benzoate, triallyl cyanurate; vinyl halides such as vinyl chloride and vinylidene chloride; aromatic mono- or divinyl compounds such as styrene, a-methylstyrene, chlorostyrene, alkylstyrene, divinylbenzene and diallyl phthalate, as well as para-styrensulfonic, vinylsulfonic, 2-(meth)acryloyloxyethylsulfonic, 2-(meth)acrylamido-2-methylpropylsulfonic acids, and mixtures thereof.

The list of monomers given is not limiting, and it should be understood that it is possible to use any monomer known to those skilled in the art which includes acrylic and/or vinyl monomers (including monomers modified with a silicone chain).

In at least certain, non-limiting exemplary embodiments carboxyl functional acrylate latex polymers may be chosen from aqueous dispersions of Methacrylic Acid/Ethyl Acrylate copolymer (INCI: Acrylates Copolymer, such as LUVIFLEX® SOFT by BASF), PEG/PPG-23/6 Dimethicone Citraconate/C10-30 Alkyl PEG-25 Methacrylate/Acrylic Acid/Methacrylic Acid/Ethyl Acrylate/Trimethylolpropane PEG-15 Triacrylate copolymer (INCI: Polyacrylate-2 Crosspolymer, such as FIXATE SUPERHOLD™ by Lubrizol), Styrene/Acrylic copolymer (such as Acudyne Shine by Dow Chemical), Ethylhexyl Acrylate/Methyl Methacrylate/Butyl Acrylate/Acrylic Acid/Methacrylic Acid copolymer (INCI: Acrylates/Ethylhexyl Acrylate Copolymer, such as Daitosol 5000SJ, Daito Kasei Kogyo), Acrylic/Acrylates Copolymer (INCI name: Acrylates Copolymer, such as DAITOSOL 5000AD, Daito Kasei Kogyo), Acrylates Copolymers, such as those known under the tradenameDermacryl AQF (Akzo Nobel), under the tradename LUVIMER® MAE (BASF), or under the tradename BALANCE CR (AKZO NOBEL), Acrylates/Hydroxyesters Acrylates Copolymer, known under the tradename ACUDYNE 180 POLYMER (Dow Chemical), Styrene/Acrylates Copolymer, known under the tradename Acudyne Bold from Dow Chemical, Styrene/Acrylates/Ammonium Methacrylate Copolymer, known under the tradename SYNTRAN PC5620 CG from Interpolymer, and mixtures thereof.

In yet further exemplary and non-limiting embodiments, the latex polymers may be chosen from carboxyl functional polyurethane latex polymers, such as aqueous polyurethane dispersions. These polyurethanes are conventionally formed by the reaction of prepolymer (i) with a coreactant (ii) to produce a carboxyl terminated or pendant polyurethane polymer. The prepolymer (i) may have the structure according to the formula (I″):

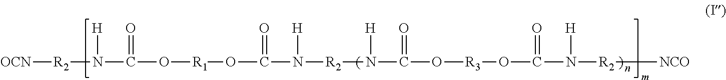

wherein R1 is chosen from bivalent radicals of a dihydroxyl functional compound, R2 is chosen from hydrocarbon radicals of an aliphatic or cycloaliphatic polyisocyanate, and R3 is chosen from radicals of a low molecular weight diol, optionally substituted with ionic groups or potential ionic groups,n ranges from about 0 to about 5, and m is greater than about 1.

Suitable dihydroxyl compounds for providing the bivalent radical R1 include those having at least two hydroxy groups, and having number average molecular weights ranging from about 700 to about 16,000, such as, for example, from about 750 to about 5000. Non-limiting examples of the high molecular weight compounds include polyester polyols, polyether polyols, polyhydroxy polycarbonates, polyhydroxy polyacetals, polyhydroxy polyacrylates, polyhydroxy polyester amides, polyhydroxy polyalkadienes and polyhydroxy polythioethers. In various embodiments, polyester polyols, polyether polyols, and polyhydroxy polycarbonates may be chosen. Mixtures of such compounds are also within the scope of the disclosure.

Optional polyisocyanates for providing the hydrocarbon-based radical R₂ include, for example, organic diisocyanates having a molecular weight ranging from about 100 to about 1500, such as about 112 to about 1000, or about 140 to about 400.

Optional diisocyanates are those chosen from the general formula R₂(NCO)₂, in which R₂ represents a divalent aliphatic hydrocarbon group comprising from about 4 to 18 carbon atoms, a divalent cycloaliphatic hydrocarbon group comprising from about 5 to 15 carbon atoms, a divalent aromatic hydrocarbon group comprising from about 7 to 15 carbon atoms, or a divalent aromatic hydrocarbon group comprising from about 6 to 15 carbon atoms.

The use of diols, for example low molecular weight diols, R3, may in at least certain embodiments allow a stiffening of the polymer chain. The expression “low molecular weight diols” means diols having a molecular weight ranging from about 50 to about 800, such as about 60 to 700, or about 62 to 200. They may, in various embodiments, contain aliphatic, alicyclic, or aromatic groups. In certain exemplary embodiments, the compounds contain only aliphatic groups. The diols that may be chosen may optionally have up to about 20 carbon atoms, and may be chosen, for example, from ethylene glycol, diethylene glycol, propane-1,2-diol, propane-1,3-diol, butane-1,4-diol, 1,3-butylene glycol, neopentyl glycol, butylethylpropanediol, cyclohexanediol, 1,4-cyclohexanedimethanol, hexane-1,6-diol, bisphenol A (2,2-bis(4-hydroxyphenyl)propane), hydrogenated bisphenol A (2,2-bis(4-hydroxycyclohexyl)-propane), and mixtures thereof. For example, R3 may be derived from neopentyl glycol.

Optionally, the low molecular weight diols may contain ionic or potentially ionic groups. Suitable low molecular weight diols containing ionic or potentially ionic groups may be chosen from those disclosed in U.S. Pat. No. 3,412,054. In various embodiments, compounds may be chosen from dimethylolbutanoic acid (DMBA), dimethylolpropionic acid (DMPA), and carboxyl-containing caprolactone polyester diol. If low molecular weight diols containing ionic or potentially ionic groups are chosen, they may, for example, be used in an amount such that less than about 0.30 meq of —COOH is present per gram of polyurethane in the polyurethane dispersion. In at least certain exemplary and non-limiting embodiments, the low molecular weight diols containing ionic or potentially ionic groups are not used.

Coreactants (ii) are compounds containing functional groups such as hydroxy or amine groups, in certain embodiments primary amine, adapted to react with isocyanate groups in preference to the carboxyl group according to the formula (II″):

X—R4-X X═OH, NH2, (II″)

wherein R₄ represents a divalent aliphatic or cycloaliphatic or aromatic hydrocarbon group, optionally substituted with ionic groups or potentially ionic groups. In various embodiments, compounds may optionally be chosen from alkylene diamines, such as hydrazine, ethylenediamine, propylenediamine, 1,4-butylenediamine and piperazine; In various embodiments, compounds may optionally be chosen from alkylene diols, such as ethylene glycol, 1,4-butanediol (1,4-BDO or BDO), 1,6-hexanediol.

As used herein, ionic or potentially ionic groups may include groups comprising ternary or quaternary ammonium groups, groups convertible into such groups, carboxyl groups, carboxylate groups, sulphonic acid groups, and sulphonate groups. At least partial conversion of the groups convertible into salt groups of the type mentioned may take place before or during the mixing with water. Special compounds may be chosen from dimethylolbutanoic acid (DMBA), dimethylolpropionic acid (DMPA), or carboxyl functional polyester comprising excess equivalents of dicarboxylic acid reacted with lesser equivalents of glycol or carboxyl-containing caprolactone polyester diol.

R1, R2, R3, R4 can have at least one carboxyl group independently.

By way of non-limiting example, such latexes include, but are not limited to, aqueous polyurethane dispersion of Isophthalic Acid/Adipic Acid/Hexylene Glycol/Neopentyl glycol/Dimethylolpropanoic Acid/Isophorone Diisocyanate copolymer (INCI name: Polyurethane-1, such as LUVISET® P.U.R, BASF), a copolymer of hexylene glycol, neopentyl glycol, adipic acid, saturated methylene diphenyldiisocyanate and dimethylolpropanoic acid monomers (INCI name: polyurethane 2), a copolymer of PPG-17, PPG-34, isophorone diisocyanate and dimethylolpropanoic acid monomers (INCI name: polyurethane 4), a copolymer of isophthalic acid, adipic acid, hexylene glycol, neopentyl glycol, dimethylolpropanoic acid, isophorone diisocyanate and bis-ethylaminoisobutyl-dimethicone monomers (INCI name: polyurethane 6), Isophorone diisocyanate, cyclohexanedimethanol, dimethylol butanoic acid, polyalkylene glycol and N-methyl diethanolamine copolymer (INCI name: polyurethane 10), Trimethylolpropane, neopentyl glycol, dimethylol propionic acid, polytetramethylene ether glycol and isocyanato methylethylbenzene copolymer (INCI name: polyurethane 12), Isophorone diisocyanate, dimethylol propionic acid, and 4,4′-isopropylidenediphenol reacted with propylene oxide, ethylene oxide and PEG/PPG-17/3 copolymer (INCI name: polyurethane 14), Isophorone diisocyanate, adipic acid, triethylene glycol and dimethylolpropionic acid copolymer (INCI name: polyurethane 15), 2-Methyl-2,4-pentanediol, polymer with 2,2-dimethyl-1,3-propanediol, hexanedioic acid, methylenedicyclohexanediisocyanate and 2,2-di(hydroxymethyl)propanoic acid, hydrolysed, tris(2-hydroxyethyl)amine salts, reaction products with 1,2-ethanediamine (INCI name: polyurethane 17), Polyurethane-27 is a complex polymer that is formed by the reaction of Polyperfluoroethoxymethoxy Difluorohydroxyethyl Ether and isophorone diisocyanate (IPDI) to form a prepolymer. The prepolymer is further reacted with the triethylamine salt of 3-hydroxy-2-(hydroxymethyl)-2-methyl-1-propionic acid (INCI name: polyurethane 27), a complex polymer formed by reacting dimethylolpropionic acid and a polyester composed of Adipic Acid, Hexylene Glycol, Neopentyl Glycol with methylene dicyclohexyldiisocyanate (SMDI) to form a prepolymer. The prepolymer is neutralized with triethylamine and then chain-extended with hydrazine (INCI name: polyurethane 33).

Fatty Acids

Carboxylic acid compound chosen from fatty acids, their salts, and mixtures thereof.

The carboxylic acid compound may generally be chosen from saturated or unsaturated carboxylic acids having carbon chains containing from 6 to 30 carbon atoms, in certain embodiments from 9 to 30 carbon atoms, and in certain embodiments from 9 to 22 carbon atoms and wherein the carbon chain is optionally substituted, for example with one or more (in particular 1 to 4) hydroxyl groups. If the fatty acids of the present disclosure are unsaturated, these compounds may comprise one to three conjugated or unconjugated carbon-carbon double bonds.

Suitable examples of the carboxylic acid compound of the present disclosure are oleic acid, linoleic acid, linolenic acid, isostearic acid, caproic acid, capric acid, caprylic acid, oleic acid, linoleic acid, linolenic acid, behenic acid, lauric acid, myristic acid, stearic acid, palmitic acid and mixtures thereof.

The carboxylic acid compound of the present disclosure may also be chosen from salts of fatty acids, in particular, alkali metal salts of fatty acids (metal soaps) and organic base salts of fatty acids.

The metal of the alkali metal salts of fatty acids includes sodium, potassium, lithium and their mixtures. The organic base salts of fatty acids may be obtained from the neutralization of fatty acids with organic bases such as ammonia, monoethanolamine or triethanolamine. Suitable examples include sodium stearate, zinc laurate, magnesium stearate, magnesium myristate, zinc stearate, potassium cocoate ammonium stearate, ammonium oleate, ammonium nonanoate, and their mixtures.

Amino Compounds

The composition according to the present disclosure comprises one or more amino compounds. The term “amino compound” is intended to mean any compound comprising at least one primary, secondary or tertiary amine or a quaternary ammonium group.

The amino compound of the present disclosure is chosen from alkyl monoamines, alkoxylated polyamines, alkoxylated monoamines, polyamines, and mixtures thereof. In certain embodiments, the amino compounds of the present disclosure do not contain silicon atoms or silicone moieties. In other embodiments, the amino compounds of the present disclosure contain silicon atoms or silicone moieties.

Alkyl Monoamines

The alkyl monoamines of the present disclosure are amino compounds having one amino group.

Non-limiting examples of preferred alkyl monoamines include aliphatic amine compounds corresponding to formula (IA) and their salts:

RN(R′)₂ (IA)

wherein

R is a hydrocarbon radical containing at least 6 carbon atoms. In addition, R can be linear or branched, acyclic or cyclic, saturated or unsaturated, aliphatic or aromatic, substituted or unsubstituted. Typically, R is a linear or branched, acyclic alkyl or alkenyl group or an alkyl phenyl group; and

the groups R′, which may be identical or different, represent H or a hydrocarbon radical containing less than 6 carbon atoms. In addition, the groups R′, which may be identical or different, are linear or branched, acyclic or cyclic, saturated or unsaturated, substituted or unsubstituted. In certain embodiments, the groups R′, which may be identical or different, are H or a methyl group.

Preferred alkyl monoamines include, but are not limited to the following examples: dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine and mixtures thereof.

Other non-limiting examples of preferred alkyl monoamines include amidoamine compounds corresponding to formula (IIA) and their salts:

RCONHR′N(R″)₂ (IIA)

wherein:

R is a hydrocarbon radical containing at least 6 carbon atoms. In addition, R can be linear or branched, acyclic or cyclic, saturated or unsaturated, aliphatic or aromatic, substituted or unsubstituted. Typically, R is a linear or branched, acyclic alkyl or alkenyl group or an alkyl phenyl group; and

R′ is a divalent hydrocarbon radical containing less than 6 carbon atoms, in certain embodiments 2 or 3 carbon atoms, and

R″ is H or a hydrocarbon radical containing less than 6 carbon atoms. In addition, R″ is linear or branched, acyclic or cyclic, saturated or unsaturated, substituted or unsubstituted. Typically, R″ is a linear or branched, acyclic alkyl or alkenyl group. In certain embodiments, R″ is H or a methyl group.

Preferred amidoamines include, but are not limited to the following examples: oleamidopropyl dimethylamine, stearamidopropyl dimethylamine, isostearamidopropyl dimethylamine, stearamidoethyl dimethylamine, lauramidopropyl dimethylamine, myristamidopropyl dimethylamine, behenamidopropyl dimethylamine, dilinoleamidopropyl dimethylamine, palmitamidopropyl dimethylamine, ricinoleamindopropyl dimethylamine, soyamidopropyl dimethylamine, wheat germamidopropyl dimethylamine, sunflowerseedamidopropyl dimethylamine, almondamidopropyl dimethylamine, avocadoamidopropyl dimethylamine, babassuamidopropyl dimethylamine, cocamidopropyl dimethylamine, minkamidopropyl dimethylamine, oatamidopropyl dimethylamine, sesamidopropyl dimethylamine, tallamidopropyl dimethylamine, brassicaamidopropyl dimethylamine, olivamidopropyl dimethylamine, palmitamidopropyl dimethylamine, stearamidoethyldiethylamine, and mixtures thereof.

Alkoxylated Polyamines

The alkoxylated polyamines of the present disclosure are chosen from amine compounds having at least two amino groups and at least one degree of alkoxylation. The alkoxylation is provided by an alkylene oxide group which is in certain embodiments chosen from ethylene oxide and propylene oxide.

Non-limiting preferred examples of suitable alkoxylated polyamines include compounds corresponding to formula (IB):

NH₂ R(R′CHCH₂O)ₓ(R′CHCH₂O)_{y}(R′CHCH₂O)_{z}RNH₂ (IB)

wherein R represents a —CH2-, —CH₂CH₂—, —CHCH₃— or —C(CH₃)₂— group, or a hydrocarbon radical containing at least 3 carbon atoms that is linear or branched, acyclic or cyclic, saturated or unsaturated, aliphatic or aromatic, substituted or unsubstituted;

x, y, and z independently of one another, represent numbers of from 0 to about 100;

R′ represents hydrogen, or an alkyl group, in certain embodiments a methyl group; and

the sum of x+y+z is at least 1.

In formula (IB), R is in certain embodiments a linear or branched, acyclic alkyl or alkenyl group or an alkyl phenyl group; x, y, and z independently of one another, in certain embodiments represent numbers ranging from 2 to 100.

Examples of the alkoxylated polyamines for use in the present disclosure which correspond to formula (IB) include, for example, tetradecyloxypropyl-1,3-diaminopropane; a C12-14 alkyl oxypropyl-1,3-diaminopropane; a C12-15 alkyloxypropyl amine and other similar materials that are commercially available from Tomah under the tradename of TOMAH DA-17.

Other examples of alkoxylated polyamines of Formula (IB) are diamine compounds belonging to the JEFFAMINE series such as the JEFFAMINE D and JEFFAMINE ED series available from Huntsman Corporation, Salt Lake City, Utah. Examples of these Jeffamine compounds are JEFFAMINE D230, JEFFAMINE D400, JEFFAMINE D2000, JEFFAMINE D4000, JEFFAMINE HK-511, JEFFAMINE ED600, JEFFAMINE ED900, and JEFFAMINE ED2003. JEFFAMINE D series compounds are amine terminated PPGs (polypropylene glycols) and JEFFAMINE ED series compounds are polyether diamine based with a predominantly PEG (polyethylene glycol) backbone.

Other non-limiting preferred examples of suitable alkoxylated polyamines in the diamine form include compounds corresponding to formula (IIB):

NH₂(CH₂)ₓOCH₂CH₂O(CH₂)ₓNH₂ (IIB)

wherein x is 2 or 3.

Examples of alkoxylated polyamines of Formula (IIB) are diamine compounds belonging to the JEFFAMINE series available from Huntsman Corporation, Salt Lake City, Utah, such as JEFFAMINE EDR148, and JEFFAMINE EDR176.

Additional non-limiting preferred examples of alkoxylated polyamines in the triamine form include compounds corresponding to formula (IIIB):

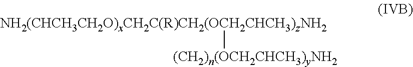

wherein R is hydrogen, —CH₂ or —C₂H₅,

n=0 or 1, and

x, y, and z independently of one another, represent numbers of from 0 to 100 and the sum of x+y+z is at least 1.

Examples of alkoxylated polyamines for use in the present disclosure which correspond to formula (IIIB) are triamine compounds belonging to the JEFFAMINE series such as the JEFFAMINE T series available from Huntsman Corporation, Salt Lake City, Utah. Examples of the JEFFAMINE T series compounds are JEFFAMINE T403, JEFFAMINE T3000, and JEFFAMINE T5000. JEFFAMINE T series compounds are triamines made by reacting PO with a triol initiator followed by aminating the terminal hydroxyl groups.

Another type of preferred alkoxylated polyamines include compounds of formulas (IVB) and (VB) hereunder:

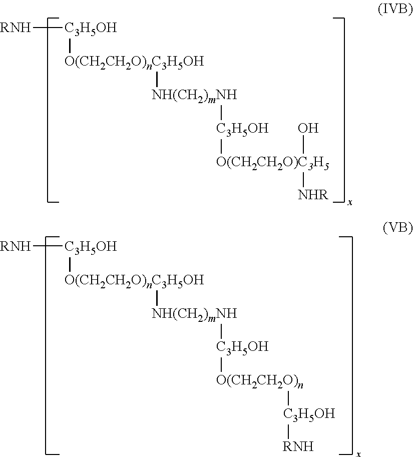

wherein

R in formula (IVB) represents the alkyl group derived from tallow and R in formula (VB) represents the alkyl group derived from coconut oil;

n in both formulas (IVB) and (VB) has a total value ranging from 10 to 20;

m in both formulas (IVB) and (VB) has a value ranging from 2 to 6; and

x in both formulas (IVB) and (VB) has a value ranging from 2 to 4.

One particular triamine alkoylated polyamine compound is JEFFAMINE T-500 polyetheramine of the formula:

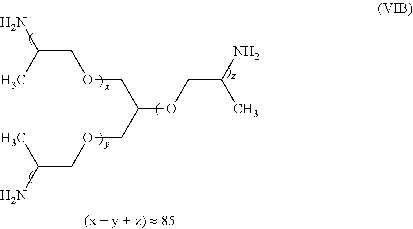

Other preferred types of alkoxylated polyamines include aminosilicones with at least one degree of alkoxylation.

Preferred examples of alkoxylated polyamines for use in the present disclosure include compounds of Formulas (IVB), (VB) and (VIB) above, such as PEG-15 Tallow Polyamine, PEG-15 Cocopolyamine, and JEFFAMINE T-500 polyetheramine, respectively.

Alkoxylated Monoamines

The alkoxylated monoamines of the present disclosure are chosen from amine compounds having at one amino groups and at least one degree of alkoxylation. The alkoxylation is provided by an alkylene oxide group which is in certain embodiments chosen from ethylene oxide and propylene oxide.

Non-limiting preferred examples of suitable alkoxylated monoamines include compounds corresponding to the formula (IC):

RN[(R′CHCH₂O)ₓH][(R′CHCH₂O)_{y}H] (IC)

wherein R is a hydrocarbon radical containing at least 6 carbon atoms. R can be linear or branched, acyclic or cyclic, saturated or unsaturated, aliphatic or aromatic, substituted or unsubstituted;

x and y, independently of one another, represent numbers of from 0 to 100 provided that the sum of x+y is >0;

the groups R′, which may be identical or different, represent hydrogen, or an alkyl group such as a methyl group.

Typically, R is a linear or branched, acyclic alkyl or alkenyl group or an alkyl phenyl group; x and y, independently of one another, are each typically a number from 0 to 30. Typically, one R′ group is hydrogen, and the other one is methyl.

Examples of preferred alkoxylated monoamines for use in the present disclosure which correspond to formula (IC) are PEG-2 Cocamine, PEG-3 Cocamine, PEG-5 Cocamine, PEG-10 Cocamine, PEG-15 Cocamine, PEG-20 Cocamine, PEG-2 Lauramine, PEG-12 Palmitamine, PEG-2 Rapeseedamine, PEG-2 Oleamine, PEG-5 Oleamine, PEG-6 Oleamine, PEG-10 Oleamine, PEG-15 Oleamine, PEG-20 Oleamine, PEG-25 Oleamine, and PEG-30 Oleamine. Other examples are alkoxylated derivatives of soyamine, stearamine and tallow amine.

Other non-limiting examples of suitable alkoxylated monoamines include compounds corresponding to formula (IIC):

RNR″[(R′CHCH₂O)ₓH] (IIC)

wherein R is a hydrocarbon radical containing at least 6 carbon atoms. R can be linear or branched, acyclic or cyclic, saturated or unsaturated, aliphatic or aromatic, substituted or unsubstituted;

x represents a number of from 1 to 100;

R′ represents hydrogen, or an alkyl group such as in particular a methyl group; and

R″ is a hydrogen or a hydrocarbon radical.

Typically, R is a linear or branched, acyclic alkyl or alkenyl group or an alkyl phenyl group; x is typically a number from 1 to 30.

When R″ in formula (IIC) is a hydrocarbon radical group, this group may be linear or branched, saturated or unsaturated, substituted or unsubstituted. The hydrocarbon radical represented by R″ may also contain an alkoxylated moiety (as defined by [(R′CHCH2O)yH]), and/or heteroatoms such as nitrogen. When R″ contains at least one alkoxylated moiety, the total number of alkoxylation in the formula may range from 1 to 120.

Examples of alkoxylated monoamines for use in the present disclosure which correspond to formula (IIC) are PEG-3 Tallow Aminopropylamine, PEG-10 Tallow Aminopropylamine, PEG-15 Tallow Aminopropylamine, and PEG-105 Behenyl Propylenediamine.

Additional non-limiting examples of alkoxylated monoamines include compounds corresponding to formula (IIIC):

R(R′CHCH₂O)ₓ(R′CHCH₂O)_{y}NH₂ (IIIC)

wherein R is a hydrocarbon radical containing at least 6 carbon atoms. R can be linear or branched, acyclic or cyclic, saturated or unsaturated, aliphatic or aromatic, substituted or unsubstituted;

x and y, independently of one another, represent numbers of from 0 to 100 with the proviso that the sum of x+y is >0;

the groups R′, which may be identical or different, represent hydrogen, or an alkyl group such as in particular a methyl group.

Typically, R is a linear or branched, acyclic alkyl or alkenyl group or an alkyl phenyl group; x and y, independently of one another, are each typically a number from 0 to 30.

Examples of alkoxylated monoamines for use in the present disclosure which correspond to formula (IIIC) are polyetheramines containing a monoamine group. These polyetheramines are commercially available from Hunstman under the

tradename JEFFAMINE (M series such as M-600, M-1000, M-2005 and M-2070) and SURFONAMINE series (B-60, B-100, B-200, L-100, L-200, L-207, L-300).

Polyamines

The polyamines may in particular be chosen from polyvinylamines, aminated polysaccharides, amine substituted polyalkylene glycols, amine substituted polyacrylate crosspolymers, amine substituted polyacrylates, amine substituted polymethacrylates, proteins, protein derivatives, amine substituted polyesters, polyamino acids, polyalkylamines, diethylene triamine, triethylenetetramine, spermidine, spermine and mixtures thereof. The polyamines for use in the present disclosure can also be chosen from aminosilicones having at least two amino groups.

The amino compound of the present disclosure selected from polyamines may be chosen from amine-containing polymers, in particular having a weight-average molecular weight ranging from 500 to 1,000,000, in certain embodiments ranging from 500 to 500,000, and preferentially ranging from 500 to 100,000. As amine-comprising polymer, use may be made of polyamines such as poly((C2-C5)alkyleneimines), and in particular polyethyleneimines and polypropyleneimines, especially poly(ethyleneimine)s; poly(allylamine); polyvinylamines and copolymers thereof, in particular with vinylamides; polyamino acids which have NH₂ groups; aminodextran; amino polyvinyl alcohol; acrylamidopropylamine-based copolymers; and chitosans.

The polyamines of the present disclosure are also in certain embodiments chosen from polyvinylamines which are generally sold under the trade name LUPAMINE or LUVIQUAT from BASF. One preferred example of such polyamines are polyvinylamines sold under the LUVIQUAT series such as Vinylamine/vinylformamide copolymer (INCI name), sold as LUVIQUAT 9030 by BASF.

The polyamines of the present disclosure may also be chosen from VinylamineNinyl Alcohol Copolymer (INCI name).

Other preferred polyamines of the present disclosure include are amine substituted polyalkylene glycols such as PEG-15 cocopolyamine and PEG-15 Tallow Polyamine and amine substituted polyacrylate crosspolymer such as the product sold under the name CARBOPOL AQUA CC polymer by Lubrizol Advanced Materials, Inc.

The polyamine compound of the present disclosure may also be chosen from proteins and protein derivatives such as wheat protein, soy protein, oat protein, collagen, and keratin protein.

In an embodiment of the present disclosure, the polyamine compound is chosen from polyamino acid compounds comprising lysine, compounds comprising arginine, compounds comprising histidine, and compounds comprising hydroxylysine. Non limiting examples include chitosan and polyamino acids such as polyarginine, polyhistidine, polylysine, and mixtures thereof.

In one preferred embodiment of the present disclosure, amino compound of the present disclosure is chosen from polyvinylamines such as Vinylamine/vinylformamide copolymer (INCI name), sold as LUVIQUAT 9030 by BASF; alkoxylated polyamines which correspond to formula (IIIB) such as JEFFAMINE T403, JEFFAMINE T3000, and JEFFAMINE 15000 (in particular, corresponds to formula (VIB); alkoxylated monoamines which correspond to formula (IIIC) such as JEFFAMINE M-600; and alkyl amines selected from aliphatic amines and amidoamines; and mixtures, thereof.

The alkyl amines selected from aliphatic amines are in certain embodiments chosen from dimethyl lauramine, dimethyl behenamine, dimethyl cocamine, dimethyl myristamine, dimethyl palmitamine, dimethyl stearamine, dimethyl tallowamine, dimethyl soyamine, stearamine, soyamine, cocamine, lauramine, palmitamine, oleamine, tallow amine, and mixtures thereof.

The alkyl amines selected from amidoamines are in certain embodiments chosen from stearamidopropyl dimethylamine, isostearamidopropyl dimethylamine, stearamidoethyl dimethylamine, lauramidopropyl dimethylamine, behenamidopropyl dimethylamine, dilinoleamidopropyl dimethylamine, babassuamidopropyl dimethylamine, cocamidopropyl dimethylamine, stearamidoethyldiethylamine, and mixtures thereof.

Amino Silicone Compound

The composition according to the invention comprises one or more amino silicone compounds. The term “amino silicone compound” is intended to mean any silicone comprising at least one primary, secondary or tertiary amine or a quaternary ammonium group.

As amino silicone compounds that may be used in the scope of the invention, the following can be cited:

polysiloxanes corresponding to formula (A):

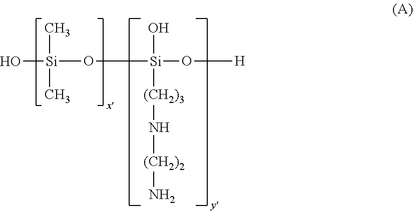

in which x′ and y′ are integers such that the weight-average molecular weight (Mw) is comprised between about 5000 and 500,000;

b) amino silicone compounds corresponding to formula (B):

R′ₐG3-a-Si(OSiG2)ₙ-(OSiGbR′2-b)ₘ-O—SiG3-a-R′ₐ (B)

in which:
G, which may be identical or different, designate a hydrogen atom, or a phenyl, OH or C1-C8 alkyl group, for example methyl, or C1-C8 alkoxy, for example methoxy, a, which may be identical or different, denote the number 0 or an integer from 1 to 3, in particular 0;
b denotes 0 or 1, and in particular 1;
m and n are numbers such that the sum (n+m) ranges from 1 to 2000 and in particular from 50 to 150, it being possible for n to denote a number from 0 to 1999 and in particular from 49 to 149, and form to denote a number from 1 to 2000 and in particular from 1 to 10;
R′, which may be identical or different, denote a monovalent radical having formula —CqH2qL in which q is a number ranging from 2 to 8 and L is an optionally quaternized amino group chosen from the following groups:

—NR″-Q-N(R″)₂

—N(R″)₂

—N+(R″)₃ A-

—N+H(R″)₂ A-

—N+H₂(R″) A-

—N(R″)-Q-N+R″H₂ A-

—NR″-Q-N+(R″)₂H A-

—NR″-Q-N+(R″)₃ A-,

in which R″, which may be identical or different, denote hydrogen, phenyl, benzyl, or a saturated monovalent hydrocarbon-based radical, for example a C1-C20 alkyl radical; Q denotes a linear or branched CrH2r group, r being an integer ranging from 2 to 6, in certain embodiments from 2 to 4; and A- represents a cosmetically acceptable ion, in particular a halide such as fluoride, chloride, bromide or iodide.

A group of amino silicone compounds corresponding to this definition (B) is represented by the silicones called “trimethylsilylamodimethicone” having formula (C):

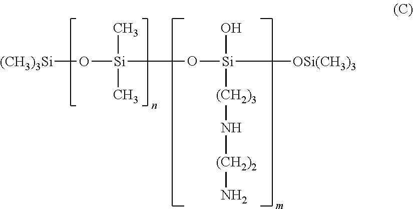

in which n and m have the meanings given above, in formula B.

Another group of amino silicone compounds corresponding to this definition is represented by silicones having the following formulae (D) or (E):

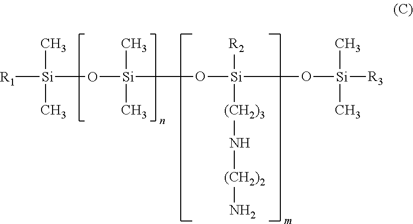

in which:
m and n are numbers such that the sum (n+m) can range from 1 to 1000, in particular from 50 to 250 and more particularly from 100 to 200, it being possible for n to denote a number from 0 to 999 and in particular from 49 to 249, and more particularly from 125 to 175, and for m to denote a number from 1 to 1000 and in particular from 1 to 10, and more particularly from 1 to 5;

R1, R2, R3, which may be identical or different, represent a hydroxy or C1-C4 alkoxy radical, where at least one of the radicals R1 to R3 denotes an alkoxy radical.

The alkoxy radical is in certain embodiments a methoxy radical.

The hydroxy/alkoxy mole ratio ranges in certain embodiments from 0.2:1 to 0.4:1 and in certain embodiments from 0.25:1 to 0.35:1 and more particularly equals 0.3:1.

The weight-average molecular weight (Mw) of the silicone ranges in certain embodiments from 2000 to 1 000 000, more particularly from 3500 to 200 000.

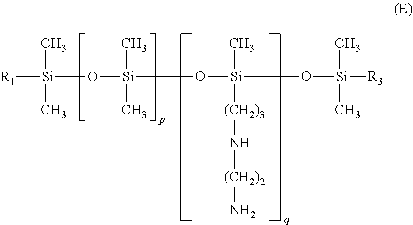

in which:
p and q are numbers such that the sum (p+q) ranges from 1 to 1000, particularly from 50 to 350, and more particularly from 150 to 250; it being possible for p to denote a number from 0 to 999 and in particular from 49 to 349, and more particularly from 159 to 239 and for q to denote a number from 1 to 1000, in particular from 1 to 10, and more particularly from 1 to 5;
R1, R2, which are different, represent a hydroxy or C1-C4 alkoxy radical, where at least one of the radicals R1 or R2 denotes an alkoxy radical.

The alkoxy radical is in certain embodiments a methoxy radical.

The hydroxy/alkoxy mole ratio ranges generally from 1:0.8 to 1:1.1 and in certain embodiments from 1:0.9 to 1:1 and more particularly equals 1:0.95.

The weight-average molecular weight (Mw) of the silicone ranges in certain embodiments from 2000 to 200 000, even more particularly 5000 to 100 000 and more particularly from 10 000 to 50 000.

Commercial products corresponding to these silicones having structure (D) or (E) may include in their composition one or more other amino silicone compounds whose structure is different than formulae (D) or (E).

A product containing amino silicone compounds having structure (D) is sold by Wacker under the name BELSIL ADM 652.

A product containing amino silicone compounds having structure (E) is sold by Wacker under the name FLUID WR 1300®.

When these amino silicone compounds are used, one particularly advantageous embodiment consists in using them in the form of an oil-in-water emulsion. The oil-in-water emulsion may comprise one or more surfactants. The surfactants may be of any nature but are in certain embodiments cationic and/or nonionic. The number-average size of the silicone particles in the emulsion generally ranges from 3 nm to 500 nanometres. In certain embodiments, in particular as amino silicone compounds having formula (E), microemulsions are used whose average particle size ranges from 5 nm to 60 nanometres (limits included) and in certain embodiments from 10 nm to 50 nanometres (limits included). Accordingly, according to the invention the microemulsions of amino silicone compound having formula (E) sold as FINISH CT 96 E® or SLM 28020® by Wacker can be used.

Another group of amino silicone compounds corresponding to this definition is represented by the following formula (F):

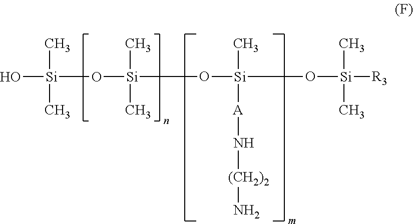

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and in certain embodiments 4 carbon atoms. This radical is in certain embodiments linear.

The weight-average molecular weight (Mw) of these amino silicone compounds ranges in certain embodiments from 2000 to 1 000 000 and even more particularly from 3500 to 200 000.

A preferred silicone of formula (F) is amodimethicone (INCI name) sold under the tradename XIAMETER® MEM-8299 Cationic Emulsion by Dow Corning.

Another group of amino silicone compounds corresponding to this definition is represented by the following formula (G):

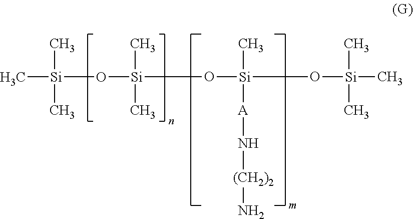

in which:
m and n are numbers such that the sum (n+m) ranges from 1 to 2000 and in particular from 50 to 150, it being possible for n to denote a number from 0 to 1999 and in particular from 49 to 149, and for m to denote a number from 1 to 2000 and in particular from 1 to 10;
A denotes a linear or branched alkylene radical containing from 4 to 8 carbon atoms and in certain embodiments 4 carbon atoms. This radical is in certain embodiments branched.

The weight-average molecular weight (Mw) of these amino silicone compounds ranges in certain embodiments from 500 to 1 000 000 and even more particularly from 1000 to 200 000.

A silicone having this formula is for example DC2-8566 Amino Fluid by Dow Corning.

c) amino silicone compounds corresponding to formula (H):

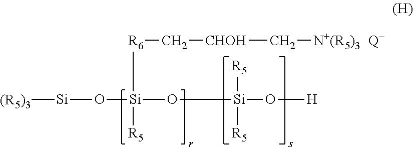

in which:
R5 represents a monovalent hydrocarbon-based radical containing from 1 to 18 carbon atoms, and in particular a C1-C18 alkyl or C2-C18 alkenyl radical, for example methyl;
R6 represents a divalent hydrocarbon-based radical, in particular a C1-C18 alkylene radical or a divalent C1-C18, for example C1-C8, alkylenoxy radical linked to the Si via an SiC bond;
Q- is an anion such as a halide ion, in particular chloride, or an organic acid salt (for example acetate);
r represents a mean statistical value from 2 to 20 and in particular from 2 to 8;
s represents a mean statistical value from 20 to 200 and in particular from 20 to 50.

Such amino silicone compounds are described more particularly in patent U.S. Pat. No. 4,185,087.

d) quaternary ammonium silicones having formula (I):

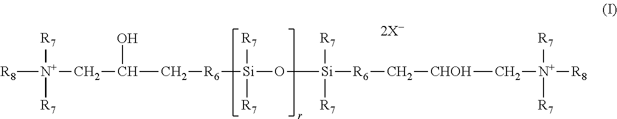

in which:
R7, which may be identical or different, represent a monovalent hydrocarbon-based radical containing from 1 to 18 carbon atoms, and in particular a C1-C18 alkyl radical, a C2-C18 alkenyl radical or a ring containing 5 or 6 carbon atoms, for example methyl;
R6 represents a divalent hydrocarbon-based radical, in particular a C1-C18 alkylene radical or a divalent C1-C18, for example C1-C8, alkylenoxy radical linked to the Si via an SiC bond;
R8, which may be identical or different, represent a hydrogen atom, a monovalent hydrocarbon-based radical containing from 1 to 18 carbon atoms, and in particular a C1-C18 alkyl radical, a C2-C18 alkenyl radical ora —R6-NHCOR7 radical;
X— is an anion such as a halide ion, in particular chloride, or an organic acid salt (for example acetate);
r represents a mean statistical value from 2 to 200 and in particular from 5 to 100;

These silicones are described, for example, in patent application EP-A 0 530 974.

e) amino silicone compounds having formula (J):

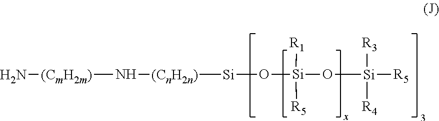

in which:
R1, R2, R3 and R4, which may be identical or different, denote a C1-C4 alkyl radical or a phenyl group;
R5 denotes a C1-C4 alkyl radical or a hydroxyl group;
n is an integer ranging from 1 to 5;
m is an integer ranging from 1 to 5;
and in which x is chosen such that the amine number is between 0.01 and 1 meq/g;

f) multiblock polyoxyalkylenated amino silicone compounds, of type (AB)n, A being a polysiloxane block and B being a polyoxyalkylenated block containing at least one amine group.

Said silicones are in certain embodiments constituted of repeating units having the following general formulae:

[—(SiMe₂O)ₓSiMe₂-R—N(R″)—R′—O(C₂H₄O)ₐ(C₃H₆O)_{b}—R′—N(H)—R—]

or alternatively

[—(SiMe₂O)ₓSiMe₂-R—N(R″)—R′—O(C₂H₄O)ₐ(C₃H₆O)_{b}—]

in which:
a is an integer greater than or equal to 1, in certain embodiments ranging from 5 to 200, more particularly ranging from 10 to 100;
b is an integer comprised between 0 and 200, in certain embodiments ranging from 4 to 100, more particularly between from 5 and 30;
x is an integer ranging from 1 to 10 000, more particularly from 10 to 5000;
R″ is a hydrogen atom or a methyl;
R, which may be identical or different, represent a divalent linear or branched C2-C12 hydrocarbon-based radical, optionally including one or more heteroatoms such as oxygen; in certain embodiments, R denotes an ethylene radical, a linear or branched propylene radical, a linear or branched butylene radical, or a —CH₂CH₂CH₂OCH(OH)CH₂— radical; preferentially R denotes a —CH₂CH₂CH₂OCH(OH)CH₂— radical;
R′, which may be identical or different, represent a divalent linear or branched C2-C12 hydrocarbon-based radical, optionally including one or more heteroatoms such as oxygen; in certain embodiments, R′ denotes an ethylene radical, a linear or branched propylene radical, a linear or branched butylene radical, or a —CH₂CH₂CH₂OCH(OH)CH₂— radical; preferentially R′ denotes —CH(CH₃)—CH₂—.

The siloxane blocks in certain embodiments represent between 50 and 95 mol % of the total weight of the silicone, more particularly from 70 to 85 mol %.

The amine content is in certain embodiments between 0.02 and 0.5 meq/g of copolymer in a 30% solution in dipropylene glycol, more particularly between 0.05 and 0.2.

The weight-average molecular weight (Mw) of the silicone is in certain embodiments comprised between 5000 and 1 000 000, more particularly between 10 000 and 200 000.

Mention may be made especially of the silicones sold under the names SILSOFT A-843 or SILSOFT A+ by Momentive.

g) the alkylamino silicone compounds corresponding to formulas (K′ and K) below:

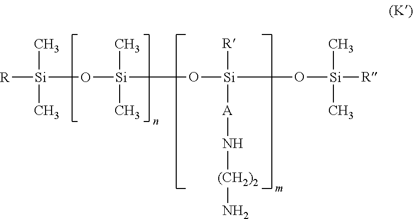

in which R, R′ and R″, which may be identical or different, each represent a C1-C4 alkyl or hydroxyl group, A represents a C3 alkylene group and m and n are such that the weight-average molecular mass of the compound is between 5000 and 500,000 approximately;

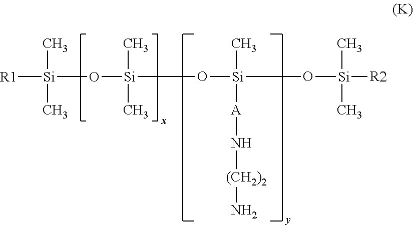

in which:

x and y are numbers ranging from 1 to 5000; in certain embodiments, x ranges from 10 to 2000 and especially from 100 to 1000; in certain embodiments, y ranges from 1 to 100;

R1 and R2, which may be identical or different, in certain embodiments identical, are linear or branched, saturated or unsaturated alkyl radicals, comprising 6 to 30 carbon atoms, in certain embodiments 8 to 24 carbon atoms and especially 12 to 20 carbon atoms;

A denotes a linear or branched alkylene radical containing from 2 to 8 carbon atoms,

In certain embodiments, A comprises 3 to 6 carbon atoms, especially 4 carbon atoms; in certain embodiments, A is branched. Mention may be made especially of the following divalent radicals: —CH₂CH₂CH₂ and —CH₂CH(CH₃)CH₂—.

In certain embodiments, R1 and R2, which may be identical or different, are saturated linear alkyl radicals comprising 6 to 30 carbon atoms, in certain embodiments 8 to 24 carbon atoms and especially 12 to 20 carbon atoms; mention may be made in particular of dodecyl, tetradecyl, pentadecyl, hexadecyl, heptadecyl, octadecyl, nonadecyl and eicosyl radicals; and preferentially, R1 and R2, which may be identical or different, are chosen from hexadecyl (cetyl) and octadecyl (stearyl) radicals.

Preferentially, the silicone is of formula (K) with:

x ranging from 10 to 2000 and especially from 100 to 1000;

y ranging from 1 to 100;

A comprising 3 to 6 carbon atoms and especially 4 carbon atoms; in certain embodiments, A is branched; and more particularly A is chosen from the following divalent radicals: CH2CH2CH2 and —CH2CH(CH3)CH2-; and

R1 and R2, which may be identical or different, being linear, saturated alkyl radicals comprising 6 to 30 carbon atoms, in certain embodiments 8 to 24 carbon atoms and especially 12 to 20 carbon atoms; chosen in particular from dodecyl, tetradecyl, pentadecyl, hexadecyl, heptadecyl, octadecyl, nonadecyl and eicosyl radicals; preferentially, R1 and R2, which may be identical or different, being chosen from hexadecyl (cetyl) and octadecyl (stearyl) radicals.

A preferred silicone of formula (K) is bis-cetearyl amodimethicone (INCI name).

Mention may be made especially of the silicone sold under the name SILSOFT AX by Momentive.

The amino silicone compounds of the present disclosure may also be chosen from polydimethylsiloxanes comprising primary amine groups at the chain end or on side chains, for example aminopropyl end or side groups, for instance those of formula (A) or (B) or (C):

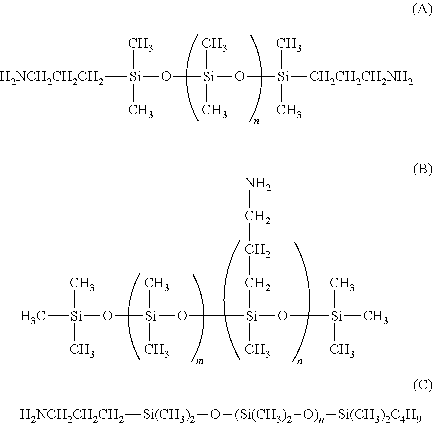

In formula (A): the value of n is such that the weight-average molecular weight of the silicone is between 500 and 55 000. As an example of aminosilicone (A), mention may be made of those sold under the names DMS-A11, DMS-A12, DMS-A15, DMS-A21, DMS-A31, DMS-A32 and DMS-A35 by the company Gelest.

In formula (B), the values of n and m are such that the weight-average molecular weight of the silicone is between 1000 and 55 000. As examples of silicone (B), mention may be made of those sold under the names AMS-132, AMS-152, AMS-162, AMS-163, AMS-191 and AMS-1203 by the company Gelest.

In formula (C), the value of n is such that the weight-average molecular weight of the silicone is between 500 and 3000. As an example of silicone (C), mention may be made of those sold under the names MCR-A11 and MCR-A12 by the company Gelest.

In certain embodiments, the amino silicone compound according to the invention is amodimethicone (INCI name) such as the amodimethicone sold under the tradename KF 8020 from the supplier Shin Etsu or the tradename SILSOFT 253 from the supplier MOMENTIVE PERFORMANCE MATERIALS. Another preferred amino silicone compound is an amodimethicone of formula (F) sold under the tradename XIAMETER® MEM-8299 Cationic Emulsion by Dow Corning.

In other embodiments, the amino silicone according to the invention is chosen from bis-cetearyl amodimethicone (sold under the name SILSOFT AX by Momentive).

If present in the composition, the above-described additives are generally present in an amount ranging up to about 95% by weight including all ranges and subranges therebetween, based on the total weight of the composition, such as up to about 50%, up to about 40%, up to about 30%, up to about 20%, up to about 15%, up to about 10%, up to about 5%, such as from about 0.001% to about 50%, or from about 0.001% to about 40%, or from about 0.001% to about 30%, or from about 0.001% to about 20%, or from about 0.001% to about 10%, by weight, based on the total weight of the composition.

Needless to say, a person skilled in the art will take care to select this or these optional additional compound(s), and/or the amount thereof, such that the advantageous properties of the composition, according to the invention, are not, or are not substantially, adversely affected by the envisaged addition.

Methods of Preparation and Methods of Use

In some embodiments, the compositions of the present disclosure are prepared by combining the polycarbodiimide, carboxysilicone polymers, and a solvent.

In other embodiments, the compositions of the present disclosure are prepared by combining the polycarbodiimide, carboxysilicone polymers, and a solvent chosen from water, organic solvents, and mixtures thereof.

In yet other embodiments, the compositions of the present disclosure are prepared by combining the polycarbodiimide, carboxysilicone polymers, and a solvent comprising water and organic solvents.

One-Step or Multi-Step Application Processes

In various embodiments, treatment regimens for keratinous substrates may include single or sequential applications of compositions comprising one or more of a polycarbodiimide and a carboxysilicone polymer. According to the various one and multi-step embodiments, the compositions of each mixture or phase may be applied once or more than once.

One-Step Application Process

In one embodiment, the composition of the present disclosure is applied onto keratinous substrates such as hair in a one-step application process. The composition for use in the one-step application is prepared by combining the ingredients, including the polycarbodiimide, carboxysilicone polymer, and solvent, resulting in a composition that is then applied onto the keratinous substrate.

In one embodiment, when the composition for a one-step application process comprises a solvent comprising water and an organic solvent, the polycarbodiimide is combined with water to form on an aqueous phase and the carboxysilicone polymer is combined with an organic solvent to form a non-aqueous phase. Both phases are then combined and agitated to form an emulsion. The formed emulsion is then applied to a keratinous substrate such as hair for treatment.

Two-Step Application Process

In other embodiments, the composition of the present disclosure is applied onto keratinous substrates such as hair in a two-step application process. In a two-step application process, the individual components (polycarbodiimide combined with a solvent and carboxysilicone polymer combined with a solvent) are applied to the keratinous substrate in a step-wise fashion in any order to treat the substrate.

In one such embodiment, the keratinous substrate is first treated with the polycarbodiimide phase, followed by treatment with the carboxysilicone polymer phase.

In another embodiment, referred to herein as a reverse-step system, compositions comprising at least one polycarbodiimide and at least one carboxysilicone polymer are prepared as separate phases, and each phase is applied separately to the keratinous substrate, such as hair. In one such embodiment, the keratinous substrate is first treated with the carboxysilicone polymer phase, followed by treatment with the polycarbodiimide phase.

Three-Step Application Process

In other embodiments, the composition of the present disclosure is applied onto keratinous substrates such as hair in a three-step application process. In a three-step application process, the individual components (polycarbodiimide combined with a solvent and carboxysilicone polymer combined with a solvent) are applied to the keratinous substrate in a step-wise fashion in any order to treat the substrate.

In one such embodiment, the keratinous substrate is first treated with the carboxysilicone polymer phase, followed by treatment with the polycarbodiimide phase, followed by treatment with the carboxysilicone polymer phase.

The application of the composition onto a keratinous substrate such as hair, according to the present disclosure, may occur at room temperature.

In other embodiments, the application of the composition according to the present disclosure, may occur at an elevated temperature (or temperatures greater than room temperature) by applying heat to the hair or exposing hair to elevated temperatures. While not so limited, heating may be provided, for example, by commonly used heating tools for example a helmet dryer or blow dryer (40° C. and above) or hot iron or flat iron (120° C.-250° C.) or steam/hot rollers.

The compositions and treatments, according to the present disclosure, provide advantageous properties to keratinous fibers. In one embodiment, the composition and treatment, according to the present disclosure, provides hydrophobicity or imparts hydrophobicity to hydrophilic or damaged keratinous fibers, such as damaged hair (platinum bleached), upon application thereto. In certain embodiments, hydrophobicity is provided at room temperature, i.e., without heating or applying heat to the hair. In other embodiments, the hydrophobicity is provided when heat is used on the hair (before or after applying the composition on the hair or during the application of the composition on the hair). And in still other embodiments, the hydrophobicity is conferred by the number and order in which applications of the compositions herein are applied, including applications of each of the polycarbodiimide and carboxysilicone polymer components, mixed and in separate forms.

In some embodiments, the hydrophobicity provided to less hydrophobic or hydrophilic keratinous fibers includes a contact angle of greater than about 50° or greater than about 60° or greater than about 70° or greater than about 80° or greater than about 90°. In some embodiments, the hydrophobicity provided to less hydrophobic or hydrophilic keratinous fibers includes a contact angle of greater than about 80° or greater than about 90° or greater than about 100° or greater than about 120° or greater than about 130° or greater than about 140°. In some embodiments the contact angle is up to about 100° or up to about 120° or up to about 130° or up to about 140°. In some embodiments wherein compositions according to the disclosure are applied according to a two or more step process, the contact angle remains at or above 100° after one or more of one shampoo, 10 shampoos, 20 shampoos, 30 shampoos, 40 shampoos, 50 shampoos, and 60 shampoos.

For example, hair treated with the inventive compositions, when heated at 50° C. for 30 minutes and then allowed to cool down for a period of time at room temperature, exhibited increased percent curl retention indicating higher resistance to high humidity and high temperature compared to hair treated with either material alone. With the use of heat (drying in the oven), the percent curl retained using the inventive composition was greater than that obtained when heat was not applied or employed on hair. Accordingly, the composition, according to the present disclosure, provides increased curling benefits when utilized, particularly when the keratinous fibers are exposed to commonly used heating tools, such as a blow dryer (60° C.) or hot iron or flat iron (120° C.-250° C.) or a heat lamp, a heat wand, or other similar devices.

The method or process of using the compositions of the present invention may depend on the type of hair being targeted and, consequently, on the specific ingredients contained in the composition used to style or shape or maintain the shape of hair.

An embodiment of the present invention is a method of styling or shaping or maintaining the shape of hair.

Another embodiment of the present invention is a method of imparting durable or long-lasting style or shape to hair comprising applying onto the hair, any one of the compositions of the present disclosure.

According to at least one embodiment, such a method comprises applying to the hair, an effective amount of any one of the compositions of the present disclosure.

The compositions of the present disclosure may be employed in an effective amount to adequately cover the surface of the fibers of the hair and to achieve a desirable or effective style or shape of the hair as well as a desirable degree of hold. The precise amount of composition to be applied onto the hair will thus depend on the degree of treatment/styling/shaping/hold desired.

An effective amount of the composition is typically from about 0.1 gram to about 50 grams, and in some applications for treatment of hair, in amounts from about 20 to 60 grams, and in yet further embodiments for an abundance of hair in amounts from about 20 to about 80 grams or more. It will thus be appreciated that the amounts applied depend on the amount or volume of keratinous substrates, such as hair, to be treated and may thus fall within lower ranges for small amounts or patches of hair to the higher ranges and beyond for large amounts or patches of hair. Typical applications are to the whole head in the case of treatment of hair. It will be understood that application to the hair typically includes working the composition through the hair.

Further disclosed herein is the use of the compositions of the present disclosure for shaping or styling hair and/or retaining a hairstyle. Also disclosed is the use of the compositions of the present disclosure for caring for the hair such as for hair repair treatments, or for reducing damage to the hair or for improving the feel of the hair by imparting hydrophobicity to the hair.

The compositions may be applied to wet or dry hair, before or after shaping. They may be used in a non-rinse fashion. In some other embodiments, the composition may be rinsed from the hair.

The hair that has been contacted with the compositions of the present invention may be air-dried and/or further styled or shaped by applying heat on the hair and/or by combing or brushing or running the fingers through the hair. Other shaping tools may be chosen from combs and brushes.

In certain embodiments, the composition is allowed to remain (leave-on time) on the keratin fibers, for example, from about 1 to about 60 minutes, or such as from about 5 to about 45 minutes, or such as from about 5 to about 30 minutes, or such as from about 10 to about 20 minutes, or such as at about 20 minutes, or such as at about 10 minutes.

The smoothing action may be accomplished by use of suitable devices for brushing or smoothing the hair include a hair brush, comb, or flat iron. The smoothing action on the hair may also include running the fingers through the hair.

A suitable applicator device is an applicator brush. It will be appreciated that while a brush is an example of a suitable applicator, particularly for hair, other applicators may be used, including but not limited to spray bottles, squeeze bottles, one and two chamber pumps, tubes, combs, and other applicators known in the art.

Heat (at a temperature of at least 40° C.) can be applied to the hair while the smoothing action is performed on the hair. The heat source can be chosen from a blow dryer, a flat iron, a hair dryer, a heat lamp, a heat wand, or other similar devices.

In addition, independently of the embodiment use, the composition present on the fibers or hair is left in place for a time, generally, from about 1 to about 60 minutes, such as from about 5 to about 45 minutes, or such as from about 5 to about 20 minutes, or such as from about 10 to about 20 minutes, or such as of about 20 minutes or such as of about 10 minutes. In alternate embodiments, the treatment times may be longer, and in some embodiments, appreciably longer, such that the application may be left on for up to 24 hours to about 48 hours.

The compositions of the present invention are easy to spread on hair.

It has surprisingly and unexpectedly discovered that the application of the composition onto the hair results in the retention of the shape or style or curl of hair or of making the hair humidity resistant. It was also surprisingly and unexpectedly discovered that the application of the composition onto the hair results in improving the quality of the hair, for example, better hair feel and appearance.

The shape/styling control, the curl retention, humidity resistant, and hair care effects obtained using the compositions and methods of the present invention may also be durable or long-lasting, i.e., wash or shampoo resistant.

As used herein, “long-lasting” or “durable” is understood to mean that the benefits imparted to hair by the compositions of the invention last over a period of time and/or over high humidity conditions and/or after one or multiple wash cycles (with water or shampoo/water or shampoo/water/conditioner/water or conditioner/water). The multiple wash cycles is understood to mean more than one wash cycle, such as two or three or four or five or six or seven or eight or nine or ten wash cycles.

Another embodiment of the present invention is method for imparting durable or long-lasting style/shape and/or curl and/or care to hair comprising (a) providing the composition of the present invention, and (b) providing instructions for applying the composition to the hair.

Instructions for applying the composition of the present invention onto keratinous substrates such as hair on the head or eyelashes may comprise directions of use of the composition for the end-user to follow. The end-user may be a consumer or cosmetologist or salon hair dresser. Directions may comprise instructing the end-user to take an amount of the composition in sufficient quantity such that the composition adequately covers the hair fibers and imparts the desired shape or style or hold to the hair fibers. Directions may additionally instruct the end-user to use a device such as a comb, brush (e.g., hair brush or brush wand), flat iron plates, blow dryer or the fingers for shaping or styling the hair or for separating the fibers of the hair. Directions may also additionally instruct the end-user to apply heat to the hair such as by blow drying the hair or using a heating device on the hair.

Instructions for applying the composition of the present invention onto keratin fibers such as hair may appear on the container (such as can, bottle or jar) holding the composition of the present invention or on the box or carton or other packaging comprising the container holding the composition.

The compositions described above are useful for application onto keratinous substrates such as hair on the head of human individuals.

Thus, the compositions of the present invention can be made into various cosmetic products such hair care products, hair styling products and make up products.

Representative types of hair care compositions, including hair cosmetic and styling compositions, of the present invention include compositions for shaping the hair, maintaining the shape of the hair, styling products (e.g., gels, creams, milks, pastes, waxes, ointments, serums, foams, hair lotions, mousses, pump-sprays, non-aerosol sprays and aerosol sprays), conditioning or protection from heat damage, leave-in hair treatments, rinse-off hair treatments, combination shampoo/styling compositions and hair volumizing compositions.

The compositions of the present invention can be in the form of an aqueous composition or an emulsion, such as a lotion or cream, and in some embodiments may be applied in another form, such as in a serum such as an anhydrous serum.

In one embodiment, the composition of the present invention is in the form of a non-aerosol spray, and in some particular embodiments containing a volatile organic solvent/compound.

In one embodiment, the composition of the present invention is in the form of a cream.

The compositions may be packaged in various forms, especially in a tube, a jar or bottles, in pump bottles, in squeeze bottles, or in aerosol containers so as to apply the composition in vaporized form or in the form of a mousse. The compositions may also impregnate applicators, especially gloves or wipes.

The composition may be applied by hand, with an applicator nozzle or actuator pump, with a container equipped with a pump, an applicator and a dispensing comb, or with an insoluble substrate impregnated with the composition.

As used herein, the process and composition disclosed herein may be used on the hair that has not been artificially dyed, pigmented or permed.

As used herein, the process and composition disclosed herein may be also used on the hair that has been artificially dyed, pigmented or permed, relaxed, straightened or other chemical process.

The compositions according to the disclosure may be prepared according to techniques that are well known to those skilled in the art.

Although the foregoing refers to various exemplary embodiments, it will be understood that the disclosure is not so limited. It will occur to those of ordinary skill in the art that various modifications may be made to the disclosed embodiments and that such modifications are intended to be within the scope of the disclosure. Where an embodiment employing a particular structure and/or configuration is illustrated in the present disclosure, it is understood that the present disclosure may be practiced with any other compatible structures and/or configurations that are functionally equivalent provided that such substitutions are not explicitly forbidden or otherwise known to be impossible to one of ordinary skill in the art.

The following examples are intended to further illustrate the present invention. They are not intended to limit the invention in any way. Unless otherwise indicated, all parts are by weight.

### EXAMPLES

The following examples are to illustrate the invention and are non-limiting. In accordance with the various examples, the evaluations and results demonstrate generally that the inventive compositions and methods confer beneficial results, including improvement to hydrophobicity.

In some embodiments, compositions are provided for treatment of a keratinous substrate comprising: at least one polycarbodiimide compound; and at least one carboxysilicone polymer selected from the group consisting of bis-terminated carboxysilicone, mono-terminated carboxysilicone, side chain carboxy-silicone, and organopolysiloxanes that contain a polyvinyl acid/ester unit; wherein the composition comprises from about 0.1 to about 40.0%, by weight, of the composition of a combined amount of the polycarbodiimide compound and the carboxysilicone polymer, wherein the combination of polycarbodiimide and carboxysilicone compounds is present in a ratio of about 1:1; and wherein the composition includes amounts of each of the polycarbodiimide compound and the carboxysilicone polymer sufficient to impart one or more of increased hydrophobicity to the keratinous substrate after application thereto and increased hold to the shape or configuration of the keratinous substrate.

According to some such embodiments, the carboysilcone compound is selected from Bis-Carboxydecyl Dimethicone, Crotonic AcidNinyl C8-12 Isoalkyl Esters/VA/Bis-Vinyldimethicone Crosspolymer, and PCA.

According to some such embodiments, the hydrophobicity imparted to the keratinous substrate includes a contact angle of greater than about 100°.

According to some such embodiments, the hydrophobicity imparted to the keratinous substrate includes a contact angle of greater than about 120°.

According to some such embodiments, the the hydrophobicity imparted to the keratinous substrate remains after one or more wash cycles.

According to some such embodiments, the composition comprises at least one additive chosen from surfactants (anionic, nonionic, cationic and amphoteric/zwtterionic), and polymers other than the polycarbodiimide and carboxysilicone polymers of the invention such as anionic polymers, nonionic polymers, amphoteric polymers, polymeric rheology modifiers, thickening and/or viscosity modifying agents, associative or non-associative polymeric thickeners, non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof, wherein the at least one additive comprising one or more rheology modifiers and thickening/viscosity-modifying agents are water-soluble or water-dispersible compounds selected from acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.

In some embodiments, a method is provided for protecting a keratinous substrate chosen from hair, eyelashes and eyebrows from extrinsic damage caused by heating, UV radiation, chemical treatment or mechanical stress, or of repairing a keratinous substrate chosen from hair, eyelashes and eyebrows following extrinsic damage caused by heating, UV radiation, chemical treatment, or mechanical stress comprising: applying to the keratinous substrate a composition according to claim 1 in an amount effective to protect or repair the keratinous substrate, wherein each of the polycarbodiimide and the carboxysilicone polymer is provided in a premix comprising at least a solvent, and the premix is applied to the keratinous substrate according to a process selected from a one step process and a multi-step process.

According to some such embodiments, the process is selected from: a one step process, wherein the composition comprising the polycarbodiimide and the carboxysilicone polymer is provided as a premix, and is prepared by combining the polycarbodiimide and carboxysilicone polymer and at least a solvent, whereby the premixed composition is applied onto the keratinous substrate; a one step process, wherein the composition is provided in separate premixes, each separately comprising the polycarbodiimide and the carboxysilicone polymer, the premixes prepared by combining the polycarbodiimide with at least water to form an aqueous phase, and separately combining the carboxysilicone polymer with at least an organic solvent to form a non-aqueous phase, whereby at the time of use, the premixed phases are combined and agitated to form an emulsion that is applied onto the keratinous substrate; a two-step process, wherein the composition is provided in separate premixes, the premixes prepared by combining the polycarbodiimide with a suitable solvent, and separately combining the carboxysilicone polymer with a suitable solvent, whereby at the time of use, each of the premixed phases is applied separately to the keratinous substrate in any order, in two sequential applications; and, a three-step process, wherein the composition is provided in separate premixes, the premixes prepared by combining the polycarbodiimide with a suitable solvent, and separately combining the carboxysilicone polymer with a suitable solvent, whereby at the time of use, each of the premixed phases is applied separately to the keratinous substrate in any order, in three sequential applications.

According to some such embodiments, the method comprises the steps of one of a two-step process and a three-step process, wherein the hydrophobicity imparted to the keratinous substrate includes a contact angle of greater than about 120°.

According to some such embodiments, the process is a two-step process comprising: the steps selected from (i) applying the carboxysilicone polymer phase, followed by applying the polycarbodiimide phase, and (ii) applying the polycarbodiimide polymer phase, followed by applying the carboxysilicone phase.

According to some such embodiments, the process is a three-step process comprising: the steps of applying the carboxysilicone polymer phase, followed by applying the polycarbodiimide phase, followed by applying the carboxysilicone polymer phase.

According to some such embodiments, the hydrophobicity imparted to the keratinous substrate includes a contact angle of greater than about 130° after five or more wash cycles.

According to some such embodiments, wherein the hydrophobicity imparted to the keratinous substrate includes a contact angle of greater than about 100° after ten or more wash cycles.

In some embodiments, an article of manufacture is provided comprising a kit containing, in separately packaged form, the kit comprising: at least one of: the composition according to claim 1 wherein the polycarbodiimide and carboxysilicone polymer are combined in a premix that comprises at least a solvent; and the composition according to claim 1 wherein the polycarbodiimide and carboxysilicone polymer are provided in in separate packages, comprising a packaged phase premix that comprises polycarbodiimide; and also comprising a packaged phase premix that comprises carboxysilicone polymer, whereby at the time of use, the premixed phases are combined one of with one another or with a suitable solvent for each of the polycarbodiimide phase and the carboxysilicone polymer phase; and optionally, one or more separately packaged solvents, optionally, one or more separately packaged components selected from surfactants (anionic, nonionic, cationic and amphoteric/zwtterionic), and polymers other than the polycarbodiimide and carboxysilicone polymers of the invention such as anionic polymers, nonionic polymers, amphoteric polymers, polymeric rheology modifiers, thickening and/or viscosity modifying agents, associative or non-associative polymeric thickeners, non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof, wherein the at least one additive comprising one or more rheology modifiers and thickening/viscosity-modifying agents are water-soluble or water-dispersible compounds selected from acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.

Procedure for preparation of compositions for application onto hair:

The compositions comprised two phases. Stock solutions of each phase were generated by stirring the active RM (raw material) in the respective solvent (water or Isododecane (IDD) or ethanol or mixtures thereof). Just before application, the desired ratio of parts (typically 1:1 actives) were weighed into vials and shaken vigorously by hand for 10 seconds/g of solution to create a milky emulsion or aqueous solution. 1 g of product was applied to 1 g of hair. In the case of 2-step treatments, designated or desired amounts of the stock solutions were applied directly to hair without pre mixing.

Hydrophobicity Evaluation—Contact Angle Measurements

Hydrophobicity of each swatch was measured via contact angle measurements using Biolin Scientific Contact Angle Tensiometer, Model C204A. A bundle of 30-50 fibers was clamped to create a flat surface. A 3 μL drop of DI (deionized) H₂O was placed on the fiber surface and the contact angle was measured for 10 seconds. The values reported below are an average of 3 measurements using the contact angle at 10 seconds. Natural, undamaged hair is hydrophobic while damaged hair (double bleached, or platinum bleached) is hydrophilic and exhibits a contact angle of 0°.

*Here and in subsequent Tables, unless otherwise indicated, “Wt % of Actives” is the total amount of the active(s) (pCDI or carboxysilicone or both) in the final composition (combination of phase A and phase B)

**Here and in subsequent Tables, unless otherwise indicated, Phase A contains a solvent comprising isododecane, Phase B contains a solvent comprising water, and the combination of Phase A and Phase B equals 100%, by weight.

TABLE 1
Representative Actives

| Generic Name | Trade Name or ingredient name |
|---|---|
| carboxysilicone polymer 1 | Silform INX |
| carboxysilicone polymer 2 | Bis-terminated Carboxy-Silicone |
| | Shin Etsu X-22-3710 |
| carboxysilicone polymer 3 | Mono-terminated Carboxy-silicone |
| | Shin Etsu X-22-162C |
| carboxysilicone polymer 4 | Side-chain Carboxy-silicone |
| | Grants Industries Gransil PCA |
| carboxysilicone polymer 5 | Branched multi-block carboxysilicone |
| | polymer (INCI name: Crotonic Acid/Vinyl |
| | C8-12 Isoalkyl Esters/VA/Bis- |
| | Vinyldimethicone Crosspolymer) |
| | Wacker Belsil P1101 |
| Carboxysilicone polymer 6 | X-22-3701E, a side-chain carboxy silicone |
| | from Shin Etsu |
| Carboxysilicone polymer 7 | SENSASIL PCA-LQ-(AP), PCA |
| | dimethicone from Croda |
| polycarbodiimide 1 | V-02-L2 |
| polycarbodiimide 2 | V-02 |
| polycarbodiimide 3 | E-03A |
| polycarbodiimide 4 | SV-02 |
| polycarbodiimide 5 | V-10 |
| polycarbodiimide 6 | SW-12G |
| polycarbodiimide 7 | E-02 |
| polycarbodiimide 8 | Picassian XL-70 |
| polycarbodiimide 9 | Picassian XL-732 |

### EXAMPLE A

Controls: Treatment of Hair with Individual Components

Platinum bleached hair swatches from the supplier IHIP were treated with the single-component treatments (1 g product/1 g hair) and dried under a helmet dryer (50° C.) for 40 minutes. Swatches were shampooed immediately and blow dried for 2 minutes on high heat. Contact angle was measured.

TABLE 2
Controls: Treatment of Hair with Individual Components

| | Wt % of Actives* | |
|---|---|---|
| | Phase A** | |
| | (carboxysilicone poymer) | |
| | Phase B** | Contact Angle |
| Sample | (polycarbodiimide) | (avg of 3) |
| Virgin Hair | n/a | ~105° |
| Platinum bleached hair | n/a | 0° |
| carboxysilicone polymer 1 | 20 wt % - A | 0° |
| | 0 wt % - B | |
| carboxysilicone polymer 1 | 5 wt % - A | 0° |
| | 0 wt % - B | |
| carboxysilicone polymer 1 | 0.5 wt % - A | 0° |
| | 0 wt % - B | |
| carboxysilicone polymer 1 | 0.25 wt % - A | 0° |
| | 0 wt % - B | |
| carboxysilicone polymer 2 | 5 wt % - A | 0° |
| | 0 wt % - B | |
| carboxysilicone polymer 3 | 5 wt % - A | 0° |
| | 0 wt % - B | |
| carboxysilicone polymer 4 | 5 wt % - A | 0° |
| | 0 wt % - B | |
| carboxysilicone polymer 5 | 5 wt % - A*** | 0° |
| | 0 wt % - B | |
| polycarbodiimide 1 | 0 wt % - A | 0° |
| | 20 wt % - B | |
| polycarbodiimide 1 | 0 wt % - A | 0° |
| | 5 wt % - B | |
| polycarbodiimide 1 | 0 wt % - A | 0° |
| | 0.25 wt % - B | |
| polycarbodiimide 2 | 0 wt % - A | 0° |
| | 0.5 wt % - B | |
| polycarbodiimide 3 | 0 wt % - A | 0° |
| | 0.5 wt % - B | |
| polycarbodiimide 4 | 0 wt % - A | 46.3° ± 8.5° |
| | 0.5 wt % - B | |
| polycarbodiimide 5 | 0 wt % - A | 0° |
| | 0.5 wt % - B | |
| polycarbodiimide 6 | 0 wt % - A | 13.67° ± 23.67 |
| | 0.5 wt % - B | |
| polycarbodiimide 7 | 0 wt % - A | 0° |
| | 0.5 wt % - B | |
| polycarbodiimide 8 | 0 wt % - A | 0° |
| | 0.5 wt % - B | |
| polycarbodiimide 9 | 0 wt % - A | 0° |
| | 0.5 wt % - B | |
| polycarbodiimide 1 | 0 wt % - A | 0° |
| | 0.5 wt % - B | |

Summary Table 2: Natural hair is hydrophobic due to a layer of 18-MEA on hair. As hair becomes more damaged, this hydrophobic barrier is lost and therefore the fiber becomes less hydrophobic and more hydrophilic. This is confirmed in the first two measurements of Table 2.

None of the single-component treatments restored the native hydrophobicity to damaged hair fibers. Polycarbodiimides 4 and 6 provide an increase in hydrophobicity, although significantly below the hydrophobicity observed in natural hair. None of the individual components of the association, from concentrations of 20 wt % active to 0.25 wt % active, result in restoration of natural hair-level hydrophobicity of the platinum bleach swatches.

Treatment of Hair with Formulas Containing Both Components (Treated in the same manner as Table 2 experiments).

TABLE 3
Treatment of Hair with polycarbodiimide 1 combined with various
silicone compounds:

| | Wt % of Actives | |
|---|---|---|
| | Phase A | |
| | (carboxysilicone poymer) | |
| | Phase B | Contact Angle |
| Components of Phase | (polycarbodiimide) | (avg of 3) |
| carboxysilicone polymer 1 - A | 20 wt % - A | 123° ± 7 |
| polycarbodiimide 1 - B | 20 wt % - B | |
| carboxysilicone polymer 1 - A | 5 wt % - A | 101° ± 2 |
| polycarbodiimide 1 - B | 5 wt % - B | |
| carboxysilicone polymer 1 - A | 0.5 wt % - A | 103° ± 10 |
| polycarbodiimide 1 - B | 0.5 wt % - B | |
| carboxysilicone polymer 1 - A | 0.25 wt % - A | 92° ± 17 |
| polycarbodiimide 1 - B | 0.25 wt % - B | |
| carboxysilicone polymer 2 - A | 0.5 wt % - A | 84° ± 3 |
| polycarbodiimide 1 - B | 0.5 wt % - B | |
| carboxysilicone polymer 3 - A | 0.5 wt % - A | 115° ± 5 |
| polycarbodiimide 1 - B | 0.5 wt % - B | |
| carboxysilicone polymer 4 - A | 0.5 wt % - A | 87.6° ± 19.9 |
| polycarbodiimide 1 - B | 0.5 wt % - B | |
| carboxysilicone polymer 5 - A | 0.5 wt % - A* | 95.0° ± 4.2 |
| polycarbodiimide 1 - B | 0.5 wt % - B | |

*For this example, phase A contains a solvent comprising ethanol.

Summary Table 3: When both components (one comprised of polycarbodiimide 1 and one a carboxylic acid functionalized silicone, a carboxysilicone polymer) are utilized in combination to form the association, a significant increase in hydrophobicity of the platinum bleach swatches is observed. The range of concentrations over which the association provides hydrophobicity is 0.5 wt % to 40 wt % total actives. While the polycarbodiimide is kept constant at polycarbodiimide 1, the carboxysilicone is altered. The hair swatch was also visually smoother and easier to comb compared to untreated platinum bleach hair.

Treatment of Hair: Altering the Species of Poly(Carbodiimide (Treated in the same manner as Table 1 experiments).

TABLE 4
Treatment of Hair with Various Poly(carbodiimides)

| | Wt % of Actives | |
|---|---|---|
| | Phase A | |
| | (carboxysilicone | |
| | poymer) | |
| | Phase B | Contact Angle |
| Components of Phase | (polycarbodiimide | (avg of 3) |
| carboxysilicone polymer 1 - A | 0.5 wt % - A | 123° ± 7 |
| polycarbodiimide 1 - B | 0.5 wt % - B | |
| carboxysilicone polymer 1 - A | 0.5 wt % - A | 70.5° ± 17.0 |
| polycarbodiimide 2 - B | 0.5 wt % - B | |
| carboxysilicone polymer 1 - A | 0.5 wt % - A | 86.12° ± 4.44 |
| polycarbodiimide 3 - B | 0.5 wt % - B | |
| carboxysilicone polymer 1 - A | 0.5 wt % - A | 102.0° ± 12.2 |
| polycarbodiimide 4 - B | 0.5 wt % - B | |
| carboxysilicone polymer 1 - A | 0.5 wt % - A | 88.67° ± 12.34 |
| polycarbodiimide 5 - B | 0.5 wt % - B | |
| carboxysilicone polymer 1 - A | 0.5 wt % - A | 103.75° ± 15.13 |
| polycarbodiimide 6 - B | 0.5 wt % - B | |
| carboxysilicone polymer 1 - A | 0.5 wt % - A | 56.33° ± 33.65 |
| polycarbodiimide 7 - B | 0.5 wt % - B | |
| carboxysilicone polymer 1 - A | 0.5 wt % - A | 5.0 sec. ± 3.5* |
| polycarbodiimide 8 - B | 0.5 wt % - B | |
| carboxysilicone polymer 1 - A | 0.5 wt % - A | 109.0° ± 1.4 |
| polycarbodiimide 9 - B | 0.5 wt % - B | |
| carboxysilicone polymer 1 - A | 0.5 wt % - A | 103.0° ± 10 |
| polycarbodiimide 1 - B | 0.5 wt % - B | |

Summary Table 4: When both actives (one comprised of polycarbodiimide and one a carboxylic acid functionalized silicone) are utilized in combination to form the association, a significant increase in hydrophobicity of the platinum bleach swatches is observed. The carboxysilicone is held constant with carboxysilicone polymer 1 and the polycarbodiimides are altered. The concentrations of actives are held at 1 wt % total active.

In the case of one association, the hydrophobicity of the association exceeded that of the individual components; however, this increased hydrophobicity was insufficient to give a 10 second droplet survival time on the swatch. In the case of this association only, the same data was instead used to measure a droplet survival time on the surface of the swatch.

TABLE 5
Treatment of Hair with An Association Based on polycarbodiimide 8

| | Wt % of Actives | |
|---|---|---|
| | Phase A | |
| | (carboxysilicone poymer) | |
| | Phase B | Contact Angle |
| Components of Phase | (polycarbodiimide | (avg of 3) |
| carboxysilicone polymer 1 - A | 0.5 wt % - A | 5.0 sec. ± 3.5 |
| polycarbodiimide 8 - B | 0.5 wt % - B | |
| polycarbodiimide 8 - B | 0.5% - B | 0.28 sec ± 0.13 |
| carboxysilicone polymer 1 - A | 0.5% - A | 0.13 sec ± 0.06 |

Summary Table 5: Treatment with the association of polycarbodiimide 8 and carboxysilicone polymer 1 increased the hydrophobicity of damaged, hydrophilic hair while the individual components did not.

Treatment of Hair: 1 Step vs. 2 Step Application Process

Applications of poly(carbodiimide) treatments can be made in a single step—as previously described—or as a sequence of two layered treatments. For example, an emulsion/dispersion/solution of a poly(carbodiimide) in water could be applied to hair, then an anhydrous treatment containing a carboxylate silicone or other compound could be applied on top. In this case, the coating may retain a layered structure, with the highly adherent poly(carbodiimide) anchoring the paired polymer to the underlying hair.

Platinum bleached hair swatches were treated with 0.5 wt % (total active) treatments containing 0.55% polycarbodiimide 1 (0.5 g treatment per g hair). Polycarbodiimide 1 treated swatches were then treated with a 5.5% solution of carboxysilicone polymer 1 in IDD (0.05 g treatment per g hair). This combination results in the deposition of the same amount of polycarbodiimide 1 and carboxysilicone polymer 1 as described in the previous section. Treated swatches were dried under a helmet dryer set to 50° C. for 40 minutes. Swatches were shampooed immediately with a conventional sulfate-based shampoo (DOP shampoo) and blow dried for 2 minutes on high heat (shampoo step or cycle involves shampooing and washing the hair). Contact angle was measured.

TABLE 6
One Step vs. Two Step application process

| | Wt % of Actives | |
|---|---|---|
| | Phase A | |
| | (carboxysilicone | |
| | poymer) | |
| | Phase B | Contact Angle |
| Components of Phase | (polycarbodiimide | (avg of 3) |
| carboxysilicone polymer 1 | 0.5 wt % | 0° |
| polycarbodiimide 1 | 0.5 wt % | 0° |
| carboxysilicone polymer 1 - A | 0.5 wt % - A | 123° ± 7 |
| polycarbodiimide 1 - B (One-step) | 0.5 wt % - B | |
| Step 1: polycarbodiimide 1 | 0.55% - A* | 61.2° ± 27.0 |
| Step 2: carboxysilicone polymer 1 | 5.5% - B* | |
| (Two-Step) | | |

*Concentrations of actives in these treatments differ from the other compared treatments; however, the amount of active material deposited to hair is the same in the 1- and 2-step applications, due to the application of unequal quantities of these solutions.

Summary Table 6: Treating hydrophilic, damaged hair with poly(carbodiimide) associations created a hydrophobic coating on hair, whether the application is performed as a one-step process or as a 2-step process with the two solutions (A and B).

Treatment of Hair: Lastingness of Carboxysilicone Polymer 1 Treatments

Hair swatches were treated in the same manner as Table 1 and 2 experiments

Hair swatches were shampooed 5× with DOP shampoo and blow dried for 2 minutes on high heat. Contact angle was measured again. This cycle was repeated so contact angle measurements were taken at 10× shampoo cycles.

TABLE 7
Lastingness of Treatments with carboxysilicone polymer 1

| | Wt % of Actives | | | |
|---|---|---|---|---|
| | Phase A | | | |
| | (carboxysilicone | | | |
| | poymer) | | 5x Shampoo | |
| | Phase B | Initial Contact | Contact | 10x Shampoo |
| Sample | (polycarbodiimide | Angle | Angle | Contact Angle |
| carboxysilicone | 5 wt % - A | 101° ± 2 | 99° ± 8 | 100°± 9 |
| polymer 1 - A | | | | |
| polycarbodiimide 1 - B | 5 wt % - B | | | |
| carboxysilicone | 0.5 wt % - A | 103° ± 10 | 108° ± 7 | 103° ± 7 |
| polymer 1 - A | | | | |
| polycarbodiimide 1 - B | 0.5 wt % - B | | | |
| carboxysilicone | 0.5 wt % - A | 103.87° ± 13.01 | 102.95° ± 15.92 | 70.42° ± 20.55 |
| polymer 1 - A | | | | |
| polycarbodiimide 3 - B | 0.5 wt % - B | | | |

Summary Table 7: Hair treated with formulas containing associations between polycarbodiimide (polycarbodiimide 1 or polycarbodiimide 3) and bis-terminated carboxysilicone (carboxysilicone polymer 1) are shown to survive 10× shampoo cycles as evidenced by the contact angle measurements.

Treatment of Hair: Lastingness of Treatments with Carboxysilicone Polymers 2, 3, and 4

Hair swatches were treated in the same manner as Table 1 and 2 experiments.

Hair swatches were shampooed 5× with DOP shampoo and blow dried for 2 minutes on high heat. Contact angle was measured again.

TABLE 8
Lastingness of Alternate Silicone Treatments

| | Wt % of Actives | | |
|---|---|---|---|
| | Phase A | | |
| | (carboxysilicone | | |
| | poymer) | Initial | |
| | Phase B | Contact | 5x Shampoo |
| Sample | (polycarbodiimide | Angle | Contact Angle |
| carboxysilicone | 0.5 wt % - A | 84° ± 3 | 92° ± 10 |
| polymer 2 - A | | | |
| polycarbodiimide 1 - B | 0.5 wt % - B | | |
| carboxysilicone | 0.5 wt % - A | 115° ± 5 | 116° ± 9 |
| polymer 3 - A | | | |
| polycarbodiimide 1 - B | 0.5 wt % - B | | |
| carboxysilicone | 0.5 wt % - A | 87.6° ± 19.9 | 110.50° ± 5.25 |
| polymer 4 - A | | | |
| polycarbodiimide 1 - B | 0.5 wt % - B | | |

Summary Table 8: Hair treated with formulas containing associations associations between polycarbodiimide 1 and bis-terminated carboxysilicone (carboxysilicone polymer 2) or mono-terminated silicone (carboxysilicone polymer 3) OR sidechain carboxylated silicone (carboxysilicone polymer 4) are shown to survive 5x shampoo cycles as evidenced by the contact angle measurements.

Treatment of Hair: Altering the heating procedure at 10% by weight of Active Material (1:1 weight ratio of carboxysilicone polymer to polycarbodiimide 1)

Platinum bleached hair swatches were treated with 10 wt % total active treatments and dried under helmet dryer following the conditions listed below. Swatches were shampooed immediately and blow dried for 2 minutes on high heat. Contact angle was measured.

The hair swatches were then shampooed 5× with DOP shampoo and blow dried for 2 minutes on high heat. Contact angle was measured again. The shampoo cycles were then continued until the 10× shampoo cycle and contact angle measurements were taken.

TABLE 9
Altering the heating procedure of 10 wt % Total Active Treatment

| | Time | | | 1.5 | | Contact |
|---|---|---|---|---|---|---|
| Wt % | in | Temperature | Pre- | min | Contact | Angle |
| of | Helmet | of Helmet | Warmed | Blow | Angle | 10 × DOP |
| actives | (min) | Dryer | Helmet | Dry | (avg of 3) | (avg of 3) |
| 10 | 40 | 50° C. | No | No | 101° ± 2 | 100° ± 9 |
| 10 | 30 | 50° C. | No | No | 111° ± 9 | Not |
| | | | | | | measured |
| 10 | 20 | 50° C. | Yes | No | 107° ± 5 | Not |
| | | | | | | measured |
| 10 | 10 | 50° C. | Yes | Yes | 114° ± 15 | 117° ± 4 |
| 10 | 15 | 75° C. | Yes | No | 124° ± 5 | 115° ± 1 |

Summary Table 9: It is evident from Table 8 that a lasting treatment using the compositions of the invention on hair was achieved at the different heating time periods under the helmet dryer (ranging from 10 to 40 minutes), at different temperatures of the helmet dryer (50°-75° C.), and with or without an additional blow-drying step. The shortest heating times under the helmet dryer (10 minutes at 50° C. and 15 minutes at 75° C.) were tested for lastingness against shampoo; after 10× shampoo cycles, both treatments still exhibited high degrees of hydrophobicity indicating survival of the treatments on the hair. The lastingness values for 30 and 20 minutes under the helmet dryer were not measured as both extremes (40/10 minutes) demonstrate lastingness, therefore it can be assumed the time points in between will demonstrate similar or the same degree of lastingness as well.

Treatment of Hair: Altering the heating procedure at 1% by weight of Active Material (1:1 weight ratio of carboxysilicone polymer to polycarbodiimide 1).

Platinum bleached hair swatches were treated with 1 wt % (total active) treatments and dried under helmet dryer following the conditions listed below. Swatches were shampooed immediately and blow dried for 2 minutes on high heat. Contact angle was measured.

The hair swatches were then shampooed 5× with DOP shampoo and blow dried for 2 minutes on high heat. Contact angle was measured again. The shampoo cycles were then continued until the 10× shampoo cycle and contact angle measurements were taken.

TABLE 10
Altering the heating procedure of 1 wt % Total Active Treatment

| | Time | Temp | | 1.5 | 48 | | Contact |
|---|---|---|---|---|---|---|---|
| Wt % | in | of | Pre- | min | hour | Contact | Angle |
| of | Helmet | Helmet | Warmed | Blow | Cure | Angle | 10 × DOP |
| actives | (m) | Dryer | Helmet | Dry | Time | (avg of 3) | (avg of 3) |
| 1 | 30 | 50° C. | No | No | No | 103° ± 2 | 96° ± 5 |
| 1 | 10 | 50° C. | Yes | Yes | Yes | 109° ± 14 | 113° ± 4 |
| 1 | 15 | 75° C. | Yes | No | No | 112° ± 8 | 98° ± 8 |

Summary Table 10: It is evident from Table 9 that a lasting treatment using the compositions of the invention on hair was achieved at the different heating time periods under the helmet dryer (ranging from 10 to 30 minutes), at different temperatures of the helmet dryer (50°-75° C.), and with or without an additional blow-drying step. After 10× shampoo cycles, all treatments still exhibited high degrees of hydrophobicity indicating survival of the treatments on the hair.

Treatment of Hair: Altering the ratio of the Actives materials while maintaining a total Active Concentration of 10 wt %

Maintaining an application of 10 wt % of total Active concentration, the ratio of actives was altered from 10:1 to 1:10 of carboxysilicone polymer 1: polycarbodiimide 1.

Treated in the same manner as Table 7, row 2 experiment: Platinum bleached hair swatches from IHIP were treated with treatments (1 g product/1 g hair) and dried under helmet dryer (50° C.) for 30 minutes. Swatches were shampooed immediately and blow dried for 2 minutes on high heat. Contact angle was measured.

TABLE 11
Altering the ratio of Actives, while Maintaining a total Active
Concentration = 10 wt %

| | Wt % of Actives | |
|---|---|---|
| Ratio of | Phase A | |
| carboxysilicone polymer | (carboxysilicone poymer) | |
| 1:polycarbodiimide 2 | Phase B | Contact Angle |
| 10 wt % Total Active | (polycarbodiimide | (avg of 3) |
| 1:10 | 0.9 wt % - A | 123° ± 5 |
| | 9 wt % - B | |
| 1:1 | 5 wt % - A | 101° ± 2 |
| | 5 wt % - B | |
| 10:1 | 9 wt % - A | 125° ± 1 |
| | 0.9 wt % - B | |

Summary Table 11: The table above shows the degrees of hydrophobicity resulting from the a ratios employed, ranging from high carbodilite concentration (1:10) to high carboxysilicone polymer concentration (10:1) while maintaining a total active concentration of 10 wt %.

Treatment of Hair: Altering the time between treatment and the first shampoo (1 wt % of total Active concentration, 1:1 ratio of carboxysilicone polymer 1 to polycarbodiimide 1)

Platinum bleached hair swatches from HIP were treated with treatments (1 g product/1 g hair) and dried under helmet dryer (50° C.) for 30 minutes. One swatch was shampooed immediately after the helmet dryer. The other swatch was allowed to rest under ambient conditions for 48 hours at which point it was shampooed 1× with DOP shampoo and blow dried for 2 minutes on high heat. Contact angle was measured.

Swatches were shampooed 5× with DOP, blow dried for 2 minutes on high heat and contact angle was measured. This cycle was repeated until 20× shampoo cycles were reached.

TABLE 12
Altering the time between treatment and the first shampoo

| Time before 1^{st} | Initial Contact | 10x DOP | 20x DOP |
|---|---|---|---|
| Shampoo | Angle | Contact Angle | Contact Angle |
| 0 h | 103° ± 2 | 96° ± 5 | 0° |
| 48 h | 120° ± 2 | 119° ± 3 | 108° ± 10 |

Summary Table 12: Increasing the length of time between the treatment and the first shampoo increases the lastingness of the treatment on the hair as measured by contact angle.

Treatment of Hair: Exposure to Chemical Treatment

Treated in the same manner as Table 5, row 3 experiment: Bleached hair swatches from IHIP were treated with 10 wt % total active treatments, 1:1 ratio of carboxysilicone polymer 1 to polycarbodiimide 1 (1 g product/1 g hair) and dried under helmet dryer (50° C.) for 10 minutes followed by 1.5 minutes of blow drying on high heat. Swatches were immediately treated with either bleach or color treatment. Contact angle was measured. (Note: The first two swatches are controls and are untreated).

Bleach Treatment

Commercial Bleach product, Commercial Hydrogen Peroxide Developer mixed in a 1:1 ratio; processing time on hair for 30 minutes. Bleach was rinsed from swatch and swatch was shampooed once with DOP shampoo and blow dried for 2 minutes.

Color Treatment

Commercial Hair Dye product, Commercial Hydrogen Peroxide Developer mixed in a 1:1 ratio; processing time on hair for 30 minutes. Bleach was rinsed from swatch and swatch was shampooed once with DOP shampoo and blow dried for 2 minutes.

TABLE 13
Exposure to chemical treatment

| Repair Treatment | Chemical Treatment | Contact Angle |
|---|---|---|
| None | Color Treatment | 0° |
| None | Bleach Treatment | 0° |
| carboxysilicone polymer 1 - A | Color Treatment | 114° ± 3 |
| polycarbodiimide 1 - B | | |
| carboxysilicone polymer 1 - A | Bleach Treatment | 103° ± 13 |
| polycarbodiimide 1 - B | | |

Summary Table 13: Exposure to chemical treatments, that is, the combination of the active components of the invention with chemical hair treatments did not alter the efficacy of the inventive compositions as evidenced by the contact angle measurements.

Thickened Phase A Formulas: Application with Active and Placebo Formulas

To aid in the application to hair in a salon setting, Phase A (previously anhydrous) was converted to a thickened water-in-oil emulsion. The new Phase A was thickened using a polymeric emulsifying thickener, Pemulen TR2 (INCI: ACRYLATES/C10-30 ALKYL ACRYLATE CROSSPOLYMER) which also contains carboxylic acid groups and can be incorporated into the crosslinked coating by reaction between poly(carbodiimide) and the thickener. At time of application, a small amount of the aqueous Phase B is added and the composition mixed to give the following final formula that is applied to hair:

TABLE 14
Active Containing Formula

| Active Thick Phase A | % by wt Active |
|---|---|
| Water | 88.7 |
| Acrylates/C10-30 Alkyl Acrylate Crosspolymer | 0.2 |
| Amino Methyl Propanol | <0.1% (to adjust to pH 6) |
| Phenoxyethanol | 0.9 |
| Ethylhexyl glycerine | 0.1 |
| polycarbodiimide 1 | 0.5 |
| Isododecane | 9.1 |
| carboxysilicone polymer 1 | 0.5 |

The relevant placebo formula replaces the polycarbodiimide-containing phase B with one composed entirely of water while utilizing the same phase A.

After mixing, the placebo composition applied to hair is:

TABLE 15
Placebo Containing Formula

| Active Thick Phase A | % by wt Active |
|---|---|
| Water | 89.2 |
| Acrylates/C10-30 Alkyl Acrylate Crosspolymer | 0.2 |
| Amino Methyl Propanol | <0.1% (to adjust to pH 6) |
| Phenoxyethanol | 0.9 |
| Ethylhexyl glycerine | 0.1 |
| Isododecane | 9.1 |
| carboxysilicone polymer 1 | 0.5 |

Application of theformuls wherein phase A is thickened was much easier than the applications of the hair treatment formulae previously described, as the increased viscosity prevented dripping and mades the product easier to spread with the hands and a comb.

Platinum bleach swatches from HIP were treated with the respective formulas (1 g product/g hair) and dried under helmet (50° C.) for 40 minutes. They were immediately shampooed 1× with DOP. Contact angle was measured.

TABLE 16
Results with Active and Placebo Formula treatments

| | Wt % of Actives | |
|---|---|---|
| | Phase A | |
| | (carboxysilicone poymer) | |
| | Phase B | |
| Sample | (polycarbodiimide | Contact Angle |
| Active Formula | 0.5 wt % - A | 120.1° ± 3.0 |
| | 0.5 wt % - B | |
| Placebo Formula | 0.5 wt % - A | 0° |
| | 0 wt % - B | |

These results show that—in addition to the improved usage and application qualities—the inventive composition, in combination with other ingredients, still imparted hydrophobicity to damaged hair that survived shampooing.

Thickened Phase B Formulas: Application with Active and Placebo Formulas

The aqueous phase of the association, containing polycarbodiimide, was thickened using hydroxyethyl cellulose (HEC). These thickened formulas were mixed with a 10 wt % solution of carboxylated silicone to afford a 1:1 ratio of actives.

The following are the active and placebo formulas for “Thick Phase B” utilized for Table 11:

TABLE 17
Active and Placebo Thick Formula treatments

| | % by wt Active |
|---|---|
| Active Thick Phase B | |
| Water | 88.2 |
| HEC | 0.9 |
| Phenoxyethanol | 0.8 |
| Ethylhexyl glycerine | 0.1 |
| polycarbodiimide 1 | 0.9 |
| Isododecane | 8.2 |
| carboxysilicone polymer 1 | 0.9 |
| Placebo Thick Phase B | |
| Water | 89.1 |
| HEC | 0.9 |
| Phenoxyethanol | 0.8 |
| Ethylhexyl glycerine | 0.1 |
| Isododecane | 8.2 |
| carboxysilicone polymer 1 | 0.9 |

Platinum bleach swatches from HIP were treated with the respective formulas (active vs. placebo) and dried under helmet (75° C.) for 15 minutes. They were immediately shampooed 1× with DOP. Contact angle was measured. Swatches were shampooed 5× with DOP, blow dried for 2 minutes on high heat and contact angle was measured. This cycle was repeated to reach 10× shampoo cycles.

TABLE 18
Application with Active and Placebo Formulas

| | Initial Contact | 5x Shampoo | 10x Shampoo |
|---|---|---|---|
| Sample | Angle | Contact Angle | Contact Angle |
| Active | 103° ± 8 | 120° ± 10 | 104° ± 12 |
| Formula | | | |
| Placebo | 0° | 0° | 0° |
| Formula | | | |

Summary Table 18: The results show that thickening the aqueous phase with HEC did not decrease the efficacy and lastingness of the treatment using the active formula.

Thickened Phase B Formulas: Application of thickened Formula to Damp Hair

Platinum bleached hair swatches from IHIP were washed 1× with DOP shampoo and towel dried. In the damp state, the hair treated with formulas described in Table 18 and heated following the conditions listed in the table: either directly blown dry or allowed to heat for 15 minutes in a 75° C. helmet dryer. Once heated, swatches were shampooed immediately with DOP shampoo and blow dried for 2 minutes on high heat. Contact angle was measured.

TABLE 19
Various Application Procedures for Thickened Formulas

| | | Time | Temp | | | | Contact | Contact |
|---|---|---|---|---|---|---|---|---|
| Wt % | | in | of | Pre- | 3 min | Contact | Angle | Angle |
| of | State | Helmet | Helmet | Warmed | Blow | Angle | 5 × | 10 × |
| actives | of Hair | (m) | Dryer | Helmet | Dry | Initial | shampoo | shampoo |
| 1 wt %—A | Damp | 15 | 75° C. | Yes | Yes | 121° ± 2 | 124° ± 3 | 124° ± 1 |
| 1 wt %—B | | | | | | | | |
| 1 wt %—A | Damp | 0 | n/a | No | Yes | 121° ± 3 | 123° ± 5 | 123° ± 5 |
| 1 wt %—B | | | | | | | | |

Summary Table 19: The thickened formula described in Table 10 may be applied to damp hair and dried in a helmet dryer (15 minutes at 75° C.) or blown dry (3 minutes on high heat). Hydrophobicity is observed over 10× shampoo cycles indicating lastingness against shampoos.

Three Reactive Species

Carbodiimide chemistry allows for multiple reaction pathways. For purposes of this technical note, the pathways of interest are:

Reaction with carboxylate, followed by rearrangement to a stable final form (N-acyl urea)

Sequential Reaction of a Carboxylate followed by an Amine (Amide)

Prior examples focus on the first pathway. However, poly(carbodiimides) allow for the incorporation of amino compounds into the covalent network through the second pathway. The following example demonstrates this alternate treatment form.

Platinum bleached hair swatches were treated in the same manner as Table 1 experiments with compositions containing either 0.25% of carboxysilicone polymer 1 and 0.25% of a second polymer containing an amine group and 0.5% of a poly(carbodiimide) or control compositions containing only the carboxylate/amine polymers or containing only the poly(carbodiimide).

TABLE 20
Third Active For 3-Component polycarbodiimide Treatments

| Functional | Trade | |
|---|---|---|
| Group | Name | INCI |
| Amine | KF 8020 | DIMETHICONE (and) AMODIMETHICONE |

TABLE 21
Treatment of Hair with 3 Actives

| | Wt % of Actives | |
|---|---|---|
| | Phase A | |
| | (carboxysilicone | |
| | poymer) | |
| | Phase B | Contact Angle |
| Components of Phase | (polycarbodiimide | (avg of 3) |
| polycarbodiimide 1 | 0 wt % - A | 0° |
| | 0.5 wt %-B | |
| carboxysilicone polymer 1 - A | 0.25 wt % - A | 0° |
| amine compound - A | 0.25 wt % - A | |
| carboxysilicone polymer 1 - A | 0.25 wt % - A | 107° ± 10 |
| amine compound - A | 0.25 wt % - A | |
| polycarbodiimide 1 - B | 0.50 wt % - B | |

Summary Table 21: Associations between polycarbodiimide 1, bis-terminated carboxysilicone (carboxysilicone polymer 1) and a third polymer containing amino functionality (KF 8020) are shown to produce shampoo-resistant hydrophobicity.

Analysis of Hair Repair Treatment—Combing Force

The smoothing properties of hair treated with 10 wt % Association of 1:1 polycarbodiimide 1: carboxysilicone 1 vs. 1 wt % Association of 1:1 polycarbodiimide 1:carboxysilicone 1 vs. those treated with DOP shampoo alone were evaluated and compared through wet combing studies. Baseline and post-treatment combing force values were taken using the Instron 3343 for product performance comparison.

Swatches were treated with the respective formulas and dried under the helmet dryer (50° C. for 30 minutes) and allowed to rest under ambient conditions for 48 hours. Swatches were then shampooed with DOP shampoo at which point each swatch was soaked in distilled water for ten (10) minutes before being combed using the Instron.

TABLE 22
Results of Combing Force Test

| Swatch Treatment | Combing Force |
|---|---|
| Control (no treatement) | 15.1 N |
| 1 wt % polycarbodiimide/carboxysilicone polymer | 0.47 N |
| 10 wt % polycarbodiimide/carboxysilicone polymer | 0.60 N |

These results show that the inventive compositions made the smoother and hair easier to comb, indicating a less damaged condition of the hair or improved hair quality.

Analysis of Hair Repair Treatment—High Humidity Curl Retention

Coiled hair is placed in the humidity chamber at 90% RH, 40° C. for 5 hours.

% Curl Retention was calculated using the formula below:

$$\% Curl Retention = \frac{( Lo - Lt )}{( Lo - Li )} * 100$$

Where: Lo=Original hair length (fully extended hair length)
Li=Initial hair length (length of hair before humidity exposure)
Lt=Length of hair after 5 hour humidity exposure

Room Temperature Application of Treatment

Regular bleached hair swatch (from IHIP, 13.5 cm long, about 0.5 g weight) is treated with solutions of 4%, by weight, of active material of test solutions (0.5 g solution/g hair). The hair was combed until the solution was uniformly distributed over the hair swatch surface. The treated hair was then rolled onto a spiral rod (0.5 in diameter) and allowed to dry at room temperature overnight.

TABLE 23
High Humidity Curl Retention of association and control samples -
applied at room Temperature

| Treatment | % Curl Retained after 5 hours |
|---|---|
| 4 wt % polycarbodiimide 1 - B | 10.14% |
| 2 wt % carboxysilicone polymer 1 - A + | 33.82% |
| 2 wt % polycarbodiimide 1 - B | |
| 4 wt % carboxysilicone polymer 1 - A | 0% |

Summary Table 23: The results indicate that hair treated with the inventive compositions at room temperature exhibited increased percent curl retention indicating higher resistance to high humidity and high temperature compared to hair treated with either material alone. The hair treated with the inventive composition displayed a higher styling property over time and at high humidity condition as indicated by the higher curl retention value. In contrast, the hair treated with either material alone demonstrated a lower curl retention value, indicating less styling hold over time and at high humidity.

Heated Application of Treatment

Swatches treated in the same method as described above. However, swatches were dried in a 50° C. oven for 30 minutes and then dried at room temperature overnight. Coiled hair was then removed from the rod for High Humidity Curl Retention measurements.

TABLE 24
High Humidity Curl Retention of association and control samples

| Treatment | % Curl Retained after 5 hours |
|---|---|
| 4 wt % polycarbodiimide 1 | 17.46% |
| 2 wt % carboxysilicone polymer 1 + | 53.73% |
| 2 wt % | |
| polycarbodiimide 1 | |
| 4 wt % carboxysilicone polymer 1 | — |

Summary Table 24: The results indicate that hair treated with the inventive compositions at 50° C. for 30 minutes and followed by drying at room temperature exhibited increased percent curl retention indicating higher resistance to high humidity and high temperature compared to hair treated with either material alone. With the use of heat (drying in the oven), the percent curl retention of the inventive composition was greater than that observed when heat was not employed. This indicates superior performance with commonly used heat tools, such as a blow dryer (60° C.), flat iron (120° C.-250° C.).

### EXAMPLE B

Procedure for Preparation of Hair Repair Treatment

Stock solutions of each phase (A and B) were generated by stirring the active RM (carboxy silicone polymer and polycarbodiimide) in the respective solvent (either water, or organic solvent such as Isododecane (IDD) or ethanol). When combining the two phases, just before application, the desired ratio of parts (typically 1:1 actives) were weighed into vial and shaken vigorously by hand for 10 seconds/g of solution to create milky emulsion. 1 g of of the milky emulsion product was applied to 1 g of hair (single component treatment). In the case of 2-step treatments, stock phase solutions were applied directly to hair without pre mixing.

Analysis of Hair Repair Treatment: Contact Angle

Hydrophobicity of each swatch was measured via contact angle measurements using Biolin Scientific Contact Angle Tensiometer, Model C204A. A bundle of 30-50 fibers was clamped to create a flat surface. A 3 uL drop of DI H₂O was placed on the fiber surface and the contact angle was measured for 10 seconds. The values reported below are an average of 3 measurements using the contact angle at 10 seconds at 12 FPS (frame rate per second).

Controls: Treatment of Hair with Individual Components

TABLE 25
The following are examples of control simplex formulas

| | | |
|---|---|---|
| Phase A | Carboxysilicone polymer 1 | 0 wt % |
| | IDD | 50 wt % |
| Phase B | Polycarbodiimide 1 | 0.5 wt % |
| | H₂O | 49.5 wt % |
| Phase A | Carboxysilicone polymer 1 | 0.5 wt % |
| | IDD | 49.5 wt % |
| Phase B | Polycarbodiimide 1 | 0 wt % |
| | H₂O | 50 wt % |

Carboxysilicone polymer 1 SILFORM INX,
Bis-Carboxydecyl Dimethicone, commercially available from Momentive;
Polycarbodiimide 1 CARBODILITE V-02-L2

Platinum bleached hair swatches from IHIP (INTERNATIONAL HAIR IMPORTERS) were treated with the single-component treatments (1 g product/1 g hair) and dried under a helmet dryer (50° C.) for 40 min. Swatches were shampooed immediately and blow dried for 2 min on high heat. Contact angle was measured in 3 different locations of the hair swatch.

TABLE 26
Controls: Treatment of Hair with Individual Components

| | Wt % of Actives | |
|---|---|---|
| | Phase A | Contact |
| | (carboxysilicone poymer) | Angle |
| | Phase B | (avg |
| Sample | (polycarbodiimide | of 3) |
| Carboxysilicone polymer 6** | 0.5 wt % - A | 0° |
| | 0 wt % - B | |
| Carboxysilicone polymer 7*** | 0.5 wt % - A | 0° |
| | 0 wt % - B | |
| Polycarbodiimide 1 CARBODILITE | 0 wt % - A | 0° |
| V-02-L2 | 0.5 wt % - B | |

**X-22-3701E, a side-chain carboxy silicone from Shin Etsu;
***SENSASIL PCA-LQ-(AP),
PCA dimethicone from Croda

Active Simplex Formulas: Treatment of Hair with Both Active Components

TABLE 27
The following are examples of active simplex formulas:

| | | |
|---|---|---|
| Phase A | Carboxysilicone polymer 6 | 0.5 wt % |
| | IDD | 49.5 wt % |
| Phase B | Polycarbodiimide 1* | 0.5 wt % |
| | H₂O | 49.5 wt % |
| Phase A | Carboxysilicone Polymer 7 | 0.5 wt % |
| | IDD | 49.5 wt % |
| Phase B | Polycarbodiimide 1* | 0.5 wt % |
| | H₂O | 49.5 wt % |

Polycarbodiimide 1* = CARBODILITE V-02-L2

Platinum bleached hair swatches from IHIP (INTERNATIONAL HAIR IMPORTERS) were treated with the single-component treatments (1 g product/1 g hair) and dried under a helmet dryer (50° C.) for 40 min. Swatches were shampooed immediately and blow dried for 2 min on high heat. Contact angle was measured in 3 different locations of the hair swatch.

TABLE 28
Treatment of Hair with V-02-L2 in combination with two different
silicone compounds

| | Wt % of Actives | |
|---|---|---|
| | Phase A | Contact Angle |
| Components of Phase | Phase B | (avg of 3) |
| Carboxysilicone polymer 6 - A | 0.5 wt % - A | 113.02° ± 0.86 |
| Polycarbodiimide 1* - B | 0.5 wt % - B | |
| Carboxysilicone polymer 7 - A | 0.5 wt % - A | 127.58° ± 3.33 |
| Polycarbodiimide 1* - B | 0.5 wt % - B | |

Polycarbodiimide 1* = CARBODILITE V-02-L2

Summary Table 28: When both actives (polycarbodiimide V-02-L2 and a carboxylic acid functionalized silicone) are utilized in combination to form the association, a significant increase in hydrophobicity of the platinum bleached hair swatches is observed. The hair swatchere were also visually smoother and easier to comb compared to untreated platinum bleach hair.

One Step, Two Step, Three Step and Reverse Step Application Regimens

In the single step (one step) system, a first phase containing a polycarbodiimide, in one embodiment comprising CARBODILITE V-02 L2, and a second phase containing a silicone carboxylate, in one embodiment comprising, Silform INX, were mixed together in a 9:1 weight ratio of the V-02-L2 phase to the Silform INX phase.

TABLE 29
Formulation for Single step system

| | wt % |
|---|---|
| First phase | |
| Polycarbodiimide 1 (CARBODILITE V-02 L2) | 2.78 |
| hydroxyethyl cellulose | 0.25 |
| hydroxypropyl guar | 0.25 |
| Phenoxyethanol | 0.9 |
| Ethylhexylglycerin | 0.09 |
| Water | QS 100 |
| Second phase | |
| Carboxysilicone Polymer 1 (Silform INX) | 10 |
| IDD | 90 |

Platinum bleached hair swatches were treated with 1% total active treatments, which contained different components for a single step (or one-step) system. During the application, hair swatch samples were washed with 20% SLES (sodium laureth sulfate) and then treated (0.4 g sample/1 g swatch) with the mixture. The samples were then put in an oven set to 50° C. for 40 minutes and left out to air-dry overnight. The samples were left for 48 hours of cure time and then washed with DOP shampoo (0.4 g DOP/1 g hair swatch). The DOP shampoo contained mainly anionic sulfate-based and amphoteric surfactants and water. The samples were left to dry and the contact angle was measured. Depending on the shampoo point, swatches were washed to that point, left to dry and measured for contact angle in triplicate.

For a 2-step/3-step/reverse system, a first formulation containing CARBODILITE V-02 L2 and a second formulation containing a silicone carboxylate, Carboxysilicone polymer 1 Silform INX were prepared.

TABLE 30
Formulations for Multi-step system and reverse system

| | wt % |
|---|---|
| First formulation | |
| Polycarbodiimide 1 (CARBODILITE V-02 L2) | 2.78 |
| hydroxyethyl cellulose | 0.25 |
| hydroxypropyl guar | 0.25 |
| Phenoxyethanol | 0.9 |
| Ethylhexylglycerin | 0.09 |
| Water | QS 100 |
| Second formulation | |
| Carboxysilicone Polymer 1 (Silform INX) | 10 |
| IDD | 80 |
| Dimethicone | 10 |

In a twostep system, platinum bleached hair swatches were treated first with the V02-L2 formulation at 0.36 g/1 g hair swatch and then with the 10% Silform INX formulation at 0.04 g/1 g hair swatch.

In a three step system, the hair swatches were treated first with the Silform INX formulation at 0.04 g/1 g hair swatch, then treated with the V-02-L2 formulation at 0.04 g/1 g hair swatch, and then lastly treated with the Silform INX formulation at 0.04 g/1 g hair swatch.

In a reverse two step system, the hair swatches were treated first with the Silform INX formulation at 0.04 g/1 g hair swatch, and then treated with the V-02-L2 formulation at 0.36 g/1 g hair swatch.

Hair swatches were initially washed with 20% SLES and then treated according to the two step or three step or reverse step system. Samples were then put in an oven set to 50° C. for 40 minutes and left out to air-dry overnight. Samples were left for 48 hours of cure time and then washed with DOP (0.4 g DOP/1 g hair swatch). Samples were left to dry and the contact angle was measured. Depending on the shampoo point, swatches were washed to that point, left to dry and measured for contact angle in triplicate.

Results on Contact Angle Measurements

TABLE 31
One Step vs Two Step Systems across Shampoos

| Sample | Average Contact Angle | Stdev. |
|---|---|---|
| 1 step after 1 shampoo | 124.12 | 8.43 |
| 1 step after 10 shampoos | 121.66 | 3.01 |
| 1 step after 20 shampoos | 111.05 | 6.38 |
| 1 step after 30 shampoos | 111.05 | 6.38 |
| 1 step after 55 shampoos | 105.63 | 1.68 |
| 1 step after 60 shampoos | 0 | 0 |
| 2 step after 1 shampoo | 122.69 | 4.85 |
| 2 step after 10 shampoos | 127.63 | 4.51 |
| 2 step after 20 shampoos | 119.02 | 1.90 |
| 2 step after 30 shampoos | 124.19 | 1.81 |
| 2 step after 55 shampoos | 118.61 | 5.26 |
| 2 step after 60 shampoos | 93.68 | 7.74 |

Summary Table 31: Treating hydrophilic, damaged hair with compositions containing associations of poly(carbodiimide) and carboxysilicone polymers created a hydrophobic coating on hair, which lasted longer over multiple shampoo treatments in the two step system compared to the coating on hair in the one step system.

TABLE 32
Reverse Two Step (reversing the order of steps) or Three Step
System

| Sample | Average Contact Angle | Stdev. |
|---|---|---|
| Reverse 2 Step 1 Shampoo | 109.04 | 4.38 |
| Reverse 2 Step 5 Shampoos | 101.99 | 1.24 |
| Reverse 2 Step 10 Shampoos | 111.70 | 2.43 |
| 3 Step 1 Shampoo | 135.87 | 1.00 |
| 3 Step 5 Shampoos | 131.27 | 0.47148009 |
| 3 Step 10 Shampoos | 141.02 | 3.41 |

Summary Table 32: Treating hydrophilic, damaged hair with the poly(carbodiimide) associations according to the reverse two step or three step systems created a hydrophobic coating on hair. At the same time, the contact angles for the hair treated according to the three step system were much greater compared to the angles for the hair treated according to the reverse two system, indicating the that the hair treated according to the three step system was very hydrophobic.

While the invention has been described with reference to certain exemplary or preferred embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

What is claimed is:

1. A composition for treatment of a keratinous substrate comprising:
at least one polycarbodiimide compound; and
at least one carboxysilicone polymer selected from the group consisting of bis-terminated carboxysilicone, mono-terminated carboxysilicone, side chain carboxy-silicone, and organopolysiloxanes that contain a polyvinyl acid/ester unit;
wherein the composition comprises from about 0.1 to about 40.0%, by weight, of the composition of a combined amount of the polycarbodiimide compound and the carboxysilicone polymer, and
wherein the composition includes amounts of each of the polycarbodiimide compound and the carboxysilicone polymer sufficient to impart one or more of increased hydrophobicity to the keratinous substrate after application thereto and increased hold to the shape or configuration of the keratinous substrate.

2. A composition according to claim 1, wherein the polycarbodiimide compound has the following formula: 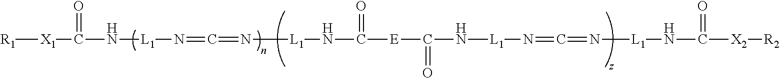
wherein X₁ and X₂, each independently, represents O, S or NH; R₁ and R₂, each independently, a hydrocarbon group containing one or more catenary or non-catenary hetero-atoms and containing linear or branched and cyclic or acyclic groups which are ionic or non-ionic segments or a partially or fully fluorinated hydrocarbon group containing one or more catenary or non-catenary hetero-atoms; n and z are, each independently, an integer of 0 to 20; L₁ represents a C₁ to C₁₈ divalent aliphatic hydrocarbon group, a C₃ to C₁₃ divalent alicyclic hydrocarbon group, a C₆ to C₁₄ divalent aromatic hydrocarbon group, a C₃ to C₁₂ divalent heterocyclic group, or a C₆ to C₁₄ divalent aromatic hydrocarbon group that is not chosen from m-tetramethylxylylene, wherein a plurality of L₁ groups may be identical to or different from one another; E is a radical selected from:
O—R₃—; S—R₄—S; and
R₅—N—R₄—N—R₅;
wherein R₃ and R₄ are, each independently, hydrocarbon radicals that may contain halogen atoms or one or more catenary or non-catenary hetero atoms, including an aromatic, cycloaliphatic, aryl and linear or branched alkyl radical and R₅ is hydrogen or a hydrocarbon radical, the hydrocarbon radical, when present, includes halogen atoms or one or more catenary or non-catenary hetero atoms.

3. A composition according to claim 2, wherein the polycarbodiimide compound is a co-polymer derived from alpha-methylstyryl-isocyanates having the following formula: 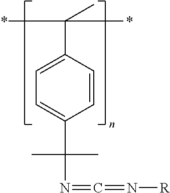
wherein R is an alkyl, cycloalkyl or aryl group having from 1 to 24 carbon atoms.

4. A composition according to claim 2, wherein the polycarbodiimide compound is a compound having the following structure: 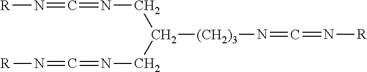
wherein R is an alkyl, cycloalkyl or aryl group.

5. A composition according to claim 4, wherein R is an alkyl, cycloaklyl or aryl group having from 1 to 24 carbon atoms.

6. A composition according to claim 1, wherein each of the polycarbodiimide compound and the carboxysilicone compound is present in a concentration, by weight, of from about 0.01% to about 20%.

7. A composition according to claim 1, wherein each of the polycarbodiimide compound and the carboxysilicone compound is present in a concentration, by weight, of from about 0.5% to about 10%.

8. A composition according to claim 1, wherein the carboysilcone compound is selected from Bis-terminated Carboxy-Silicone, Bis-Carboxydecyl Dimethicone, Mono-terminated Carboxy-silicone, Side-chain Carboxy-silicone, Branched multi-block carboxysilicone polymer (Crotonic Acid/Vinyl C8-12 Isoalkyl Esters/VA/Bis-Vinyldimethicone Crosspolymer), and PCA dimethicone.

9. A composition according to claim 1, wherein the combination of polycarbodiimide and carboxysilicone compounds is present in a ratio of about 1:1.

10. A composition according to claim 1, wherein the combination of polycarbodiimide and carboxysilicone compounds is present in a ratio of other than 1:1.

11. A composition according to claim 1, wherein the hydrophobicity imparted to the keratinous substrate includes a one or more of a contact angle of greater than about 100° and a contact angle of greater than about 120°.

12. A composition according to claim 1, wherein the hydrophobicity imparted to the keratinous substrate remains after one or more wash cycles.

13. A composition according to claim 1, comprising at least one additive chosen from surfactants (anionic, nonionic, cationic and amphoteric/zwtterionic), and polymers other than the polycarbodiimide and carboxysilicone polymers of the invention such as anionic polymers, nonionic polymers, amphoteric polymers, polymeric rheology modifiers, thickening and/or viscosity modifying agents, associative or non-associative polymeric thickeners, non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof, wherein the at least one additive comprising one or more rheology modifiers and thickening/viscosity-modifying agents are water-soluble or water-dispersible compounds selected from acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.

14. A method of protecting a keratinous substrate chosen from hair, eyelashes and eyebrows from extrinsic damage caused by heating, UV radiation, chemical treatment or mechanical stress, or of repairing a keratinous substrate chosen from hair, eyelashes and eyebrows following extrinsic damage caused by heating, UV radiation, chemical treatment, or mechanical stress comprising:
applying to the keratinous substrate a composition according to claim 1 in an amount effective to protect or repair the keratinous substrate,
wherein each of the polycarbodiimide and the carboxysilicone polymer is provided in a premix comprising at least a solvent, and the premix is applied to the keratinous substrate according to a process selected from a one step process and a multi-step process.

15. A method according to claim 14, wherein the process is selected from:
a one step process, wherein the composition comprising the polycarbodiimide and the carboxysilicone polymer is provided as a premix, and is prepared by combining the polycarbodiimide and carboxysilicone polymer and at least a solvent, whereby the premixed composition is applied onto the keratinous substrate;
a one step process, wherein the composition is provided in separate premixes, each separately comprising the polycarbodiimide and the carboxysilicone polymer, the premixes prepared by combining the polycarbodiimide with at least water to form an aqueous phase, and separately combining the carboxysilicone polymer with at least an organic solvent to form a non-aqueous phase, whereby at the time of use, the premixed phases are combined and agitated to form an emulsion that is applied onto the keratinous substrate;
a two-step process, wherein the composition is provided in separate premixes, the premixes prepared by combining the polycarbodiimide with a suitable solvent, and separately combining the carboxysilicone polymer with a suitable solvent, whereby at the time of use, each of the premixed phases is applied separately to the keratinous substrate in any order, in two sequential applications; and,
a three-step process, wherein the composition is provided in separate premixes, the premixes prepared by combining the polycarbodiimide with a suitable solvent, and separately combining the carboxysilicone polymer with a suitable solvent, whereby at the time of use, each of the premixed phases is applied separately to the keratinous substrate in any order, in three sequential applications.

16. A method according to claim 15, comprising the steps of one of a two-step process and a three-step process, wherein the hydrophobicity imparted to the keratinous substrate includes a contact angle of greater than about 120°.

17. A method according to claim 16, wherein the process is a two-step process comprising: the steps selected from (i) applying the carboxysilicone polymer phase, followed by applying the polycarbodiimide phase, and (ii) applying the polycarbodiimide polymer phase, followed by applying the carboxysilicone phase.

18. A method according to claim 16, wherein the process is a three-step process comprising: the steps of applying the carboxysilicone polymer phase, followed by applying the polycarbodiimide phase, followed by applying the carboxysilicone polymer phase.

19. A method according to claim 18, wherein the hydrophobicity imparted to the keratinous substrate includes one or more of a a contact angle of greater than about 130° after five or more wash cycles, and a contact angle of greater than about 100° after ten or more wash cycles.

20. An article of manufacture comprising a kit containing, in separately packaged form, the kit comprising:
at least one of:
the composition according to claim 1 wherein the polycarbodiimide and carboxysilicone polymer are combined in a premix that comprises at least a solvent; and
the composition according to claim 1 wherein the polycarbodiimide and carboxysilicone polymer are provided in in separate packages, comprising a packaged phase premix that comprises polycarbodiimide; and also comprising a packaged phase premix that comprises carboxysilicone polymer, whereby at the time of use, the premixed phases are combined one of with one another or with a suitable solvent for each of the polycarbodiimide phase and the carboxysilicone polymer phase; and
optionally, one or more separately packaged solvents,
optionally, one or more separately packaged components selected from surfactants (anionic, nonionic, cationic and amphoteric/zwtterionic), and polymers other than the polycarbodiimide and carboxysilicone polymers of the invention such as anionic polymers, nonionic polymers, amphoteric polymers, polymeric rheology modifiers, thickening and/or viscosity modifying agents, associative or non-associative polymeric thickeners, non-polymeric thickeners, nacreous agents, opacifiers, dyes or pigments, fragrances, mineral, plant or synthetic oils, waxes including ceramides, vitamins, UV-screening agents, free-radical scavengers, antidandruff agents, hair-loss counteractants, hair restorers, preserving agents, pH stabilizers and solvents, and mixtures thereof, wherein the at least one additive comprising one or more rheology modifiers and thickening/viscosity-modifying agents are water-soluble or water-dispersible compounds selected from acrylic polymers (in particular, Acrylates/C10-30 Alkyl Acrylate Crosspolymer, carbomers, acrylate copolymers, acrylate crosspolymers), non-acrylic polymers, starch, saccharide-based polymers (e.g., guar, guar gums), cellulose-based polymers (in particular, hydroxyethylcellulose, cellulose gums, alkyl hydroxyethyl cellulose, carboxylic acid containing celluloses/carbohydrates), non-polymeric and polymeric gelling agents, silica particles, clay, hyaluronic acid, alginic acid, and mixtures thereof.
